(19) United States
(12) Patent Application Publication
(10) Pub. No.: US 2021/0238176 A1
(43) Pub. Date: Aug. 5, 2021

(54) PESTICIDALLY-ACTIVE BICYCLIC HETEROAROMATIC COMPOUNDS

(71) Applicant: SYNGENTA CROP PROTECTION AG, Basel (CH)

(72) Inventors: Aurelien BIGOT, Stein (CH); Jürgen Harry SCHAETZER, Stein (CH); Pierre Joseph Marcel JUNG, Stein (CH); André STOLLER, Stein (CH); Julien Daniel Henri GAGNEPAIN, Stein (CH); Roger Graham HALL, Stein (CH); Stefano RENDINE, Stein (CH); Nicola COMPAGNONE, Stein (CH)

(73) Assignee: SYNGENTA CROP PROTECTION AG, Basel (CH)

(21) Appl. No.: 17/266,208

(22) PCT Filed: Jul. 31, 2019
(86) PCT No.: PCT/EP2019/070666

§ 371 Date: Feb. 5, 2021

(30) Foreign Application Priority Data
Aug. 7, 2018 (EP) 18187790.3
May 6, 2019 (EP) 19172837.7

(51) Int. Cl. C07D 471/04; A01N 43/90; A01N 25/26; A01N 25/04; A01N 25/08; A01N 25/30; A01N 25/14

(52) U.S. Cl. CPC C07D 471/04; A01N 43/90; A01N 25/26; A01N 25/14; A01N 25/08; A01N 25/30; A01N 25/04

(57) ABSTRACT

A compound of formula (I) wherein the substituents are as defined in claim 1, and the agrochemically acceptable salts, stereoisomers, enantiomers, tautomers and N-oxides of those compounds, can be used as insecticides.

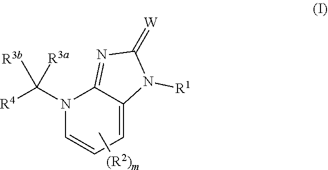

The present invention relates to pesticidally active, and in particular, insecticidally active, fused bicyclic heterocyclic compounds, to compositions comprising those compounds, and to their use for controlling animal pests (including arthropods and in particular insects or representatives of the order Lepidoptera and Hemiptera).

Insecticidally-active fused bicyclic heteroaromatic compounds are known, for example, from WO 2013/149903, WO 2007/115647, WO 2012/136751, WO 2013/144088, WO 2013/150115, WO 2012/152741 and WO 2014/076272.

It has now been found that further fused bicyclic heteroaromatic compounds have insecticidal properties.

According to the present invention, there is provided a compound of Formula (I):

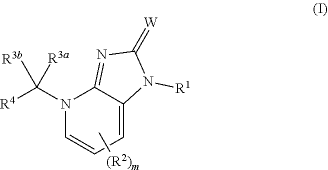

wherein:

W is O or S;

R¹ is phenyl or naphthyl, each optionally: (i) mono- or polysubstituted by a substituent independently selected from U₁ₐ, (ii) mono- or disubstituted by a substituent independently selected from U_{1b}, or (iii) mono- or disubstituted by a substituent independently selected from U₁ₐ and monosubstituted by a substituent selected from U_{1b}; or

R¹ is a 5- to 12-membered heteroaromatic ring system or a 3- to 12-membered saturated or partially saturated heterocyclic ring system, wherein the ring system is monocyclic or polycyclic and comprises 1 to 4 heteroatoms selected from nitrogen, oxygen and sulfur, with the proviso that each ring system cannot contain more than 2 oxygen or sulfur atoms, and wherein each ring system is optionally: (i) mono- or polysubstituted by a substituent independently selected from U₁ₐ, (ii) mono- or disubstituted by a substituent independently selected from U_{1b}, or (iii) mono- or disubstituted by a substituent independently selected from U₁ₐ and monosubstituted by a substituent selected from U_{1b};

U₁ₐ is independently selected from halogen, C₁-C₆alkyl, C₁-C₆haloalkyl, C₁-C₆alkoxy and C₁-C₆haloalkoxy;

U_{1b} is independently selected from nitro, cyano, amino, hydroxyl, —SCN, —CO₂H, C₃-C₆cycloalkyl, C₃-C₆halocycloalkyl, C₃-C₆cycloalkyl-C₁-C₄alkyl, C₃-C₆halocycloalkyl-C₁-C₄alkyl, C₁-C₄alkoxy-C₁-C₄alkyl, C₁-C₄alkoxy-C₁-C₄alkoxy, cyano-C₁-C₄alkyl, cyano-C₁-C₄haloalkyl, C₂-C₆alkenyl, C₂-C₆haloalkenyl, C₂-C₆alkynyl, C₂-C₆haloalkynyl, C₁-C₄haloalkoxy-C₁-C₄alkyl, C₁-C₆alkylsulfanyl, C₁-C₆alkylsulfinyl, C₁-C₆alkylsulfonyl, C₁-C₆haloalkylsulfanyl, C₁-C₆haloalkylsulfinyl, C₁-C₆haloalkylsulfonyl, C₁-C₆alkylcarbonyl, C₁-C₆alkoxycarbonyl, C₁-C₆haloalkylcarbonyl, C₁-C₆haloalkoxycarbonyl, (C₁-C₆alkyl)N(H)—, (C₁-C₆alkyl)₂N—, (C₃-C₆cycloalkyl)N(H)—, (C₃-C₆cycloalkyl)₂N—, C₁-C₆alkylcarbonylamino, C₃-C₆cycloalkylcarbonylamino, C₁-C₆haloalkylcarbonylamino, C₃-C₆halocycloalkylcarbonylamino, C₁-C₆alkylaminocarbonyl, C₃-C₆cycloalkylaminocarbonyl, C₁-C₆haloalkylaminocarbonyl, C₃-C₆halocycloalkylaminocarbonyl, C₃-C₆cycloalkylcarbonyl, C₃-C₆halocycloalkylcarbonyl, —SF₅, —NHS(O)₂ C₁-C₄alkyl, formyl or —C(O)NH₂; or

U_{1b} is phenyl optionally mono- or disubstituted by a group independently selected from U₂; or

U_{1b} is a 5- or 6-membered heteroaromatic ring or a 5- or 6-membered saturated or partially saturated heterocyclic ring, wherein each ring comprises 1 to 4 heteroatoms selected from nitrogen, oxygen and sulfur, with the proviso that each ring cannot contain more than 2 oxygen or sulfur atoms, and wherein each ring is optionally mono- or disubstituted by a group independently selected from U₂;

U² is halogen, C₁-C₆alkyl, C₁-C₆haloalkyl, C₁-C₆alkoxy, C₁-C₆haloalkoxy, nitro, cyano, amino, hydroxyl, —SCN, —CO₂H, C₃-C₆cycloalkyl, C₃-C₆halocycloalkyl, C₃-C₆cycloalkyl-C₁-C₄alkyl, C₃-C₆halocycloalkyl-C₁-C₄alkyl, C₁-C₄alkoxy-C₁-C₄alkyl, C₁-C₄alkoxy-C₁-C₄alkoxy, cyano-C₁-C₄alkyl, cyano-C₁-C₄haloalkyl, C₂-C₆alkenyl, C₂-C₆haloalkenyl, C₂-C₆alkynyl, C₂-C₆haloalkynyl, C₁-C₄haloalkoxy-C₁-C₄alkyl, C₁-C₆alkylsulfanyl, C₁-C₆alkylsulfinyl, C₁-C₆alkylsulfonyl, C₁-C₆haloalkylsulfanyl, C₁-C₆haloalkylsulfinyl, C₁-C₆haloalkylsulfonyl, C₁-C₆alkylcarbonyl, C₁-C₆alkoxycarbonyl, C₁-C₆haloalkylcarbonyl, C₁-C₆haloalkoxycarbonyl, —SF₅ or —C(O)NH₂;

m is 0, 1 or 2;

R² is independently selected from halogen, cyano, amino, hydroxyl, C₁-C₆alkyl, C₁-C₆haloalkyl, C₁-C₆haloalkoxy, C₁-C₆alkoxy, C₂-C₆alkenyl, C₂-C₆haloalkenyl, C₂-C₆alkynyl, C₂-C₆haloalkynyl, C₃-C₆cycloalkyl, C₃-C₆halocycloalkyl, C₁-C₆alkylsulfanyl, C₁-C₆alkylsulfinyl, C₁-C₆alkylsulfonyl, C₁-C₆haloalkylsulfanyl, C₁-C₆haloalkylsulfinyl and C₁-C₆haloalkylsulfonyl;

R^{3a} and R^{3b} are independently selected from hydrogen, halogen, C₁-C₄alkyl, C₁-C₄haloalkyl, C₁-C₄alkoxy, C₁-C₄haloalkoxy and cyano;

R₄ is selected from one of Y1 to Y7;

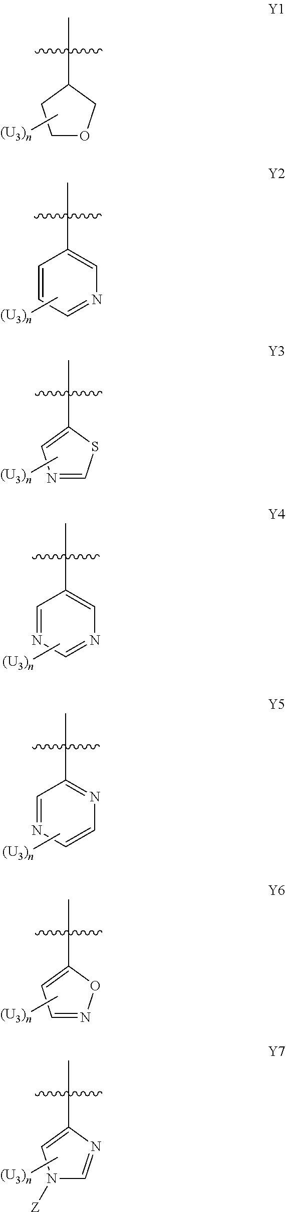

wherein, n is 0, 1, 2, or 3;

Z is hydrogen, C₁-C₄alkyl, C₁-C₄haloalkyl, C₁-C₄alkoxy or C₁-C₄haloalkoxy; and

U₃ is independently selected from halogen, cyano, nitro, hydroxyl, amino, C₁-C₄alkyl, C₁-C₄haloalkyl, C₁-C₄alkoxy, C₁-C₄haloalkoxy, C₁-C₄haloalkoxy-C₁-C₄alkyl, C₁-C₄alkoxy-C₁-C₄alkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, formyl, cyclopropyl, C₁-C₆alkylcarbonyl or C₃-C₆cycloalkylcarbonyl;

or an agrochemically acceptable salt, stereoisomer, enantiomer, tautomer or N-oxide thereof.

Surprisingly, it has been found that the novel compounds of Formula (I) have, for practical purposes, a very advantageous level of biological activity for protecting plants against insects.

According to a second aspect of the invention, there is provided a compound of Formula (II):

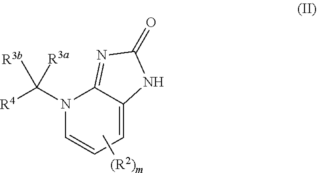

wherein m, R², R^{3a}, R^{3b} and R⁴ correspond to the same definitions as for the compounds of Formula (I), with the proviso that the compound of Formula (II) is not 4-[(6-chloro-3-pyridyl)methyl]-1H-imidazo[4,5-b]pyridin-2-one.

According to a third aspect of the invention, there is provided an agrochemical composition comprising an insecticidally, acaricidally, nematicidally or molluscicidally effective amount of a compound of formula (I) as defined according to the invention.

According to a fourth aspect of the invention, there is provided a method of controlling insects, acarines, nematodes or molluscs which comprises applying an insecticidally, acaricidally, nematicidally or molluscicidally effective amount of a compound of formula (I) as defined according to the invention, or a composition comprising this compound as active ingredient, to a pest, a locus of pest (preferably a plant), to a plant susceptible to attack by a pest or to plant a propagation material thereof (such as a seed). According to this particular aspect of the invention, the method may exclude methods for the treatment of the human or animal body by surgery or therapy.

According to a fifth aspect of the invention, there is provided the use of a compound according to Formula (I) as an insecticide, acaricide, nematicide or molluscicide. According to this particular aspect of the invention, the use may exclude methods for the treatment of the human or animal body by surgery or therapy.

As used herein, the term “halogen” or “halo” refers to fluorine (fluoro), chlorine (chloro), bromine (bromo) or iodine (iodo), preferably fluorine, chlorine or bromine.

As used herein, cyano means a —CN group.

As used herein, the term “hydroxyl” or “hydroxy” means an —OH group.

As used herein, amino means an —NH₂ group.

As used herein, nitro means an —NO₂ group.

As used herein, formyl means a —C(O)H group.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁-C₄alkyl, C₁-C₃alkyl and C₁-C₂alkyl are to be construed accordingly. Examples of C₁-C₆alkyl include, but are not limited to, methyl, ethyl, n-propyl, 1-methylethyl (isopropyl), n-butyl, and 1,1-dimethylethyl (f-butyl). A “C₁-C₄alkylene” group refers to the corresponding definition of C₁-C₄alkyl, except that such radical is attached to the rest of the molecule by two single bonds. Examples of C₁-C₄alkylene, are —CH₂— and —CH₂CH₂—.

As used herein, the term “C₁-C₆haloalkyl” refers to a C₁-C₆alkyl radical as generally defined above substituted by one or more of the same or different halogen atoms. Examples of C₁-C₆haloalkyl include, but are not limited to fluoromethyl, fluoroethyl, difluoromethyl, trifluoromethyl, 2,2-difluoroethyl, 2,2,2-trifluoroethyl, and 3,3,3-trifluoropropyl.

As used herein, the term “C₁-C₆alkoxy” refers to a radical of the formula RₐO— where Rₐ is a C₁-C₆alkyl radical as generally defined above. The term “C₁-C₄alkoxy” should be construed accordingly. Examples of C₁-C₆alkoxy include, but are not limited to, methoxy, ethoxy, propoxy, iso-propoxy, and t-butoxy.

As used herein, the term “C₁-C₆haloalkoxy” refers to a C₁-C₆alkoxy group as defined above substituted by one or more of the same or different halogen atoms. C₁-C₄haloalkoxy is to be construed accordingly. Examples of C₁-C₆haloalkoxy include, but are not limited to, fluoromethoxy, difluoromethoxy, fluoroethoxy, trifluoromethoxy, and trifluoroethoxy.

As used herein, the term “C₂-C₆alkenyl” refers to a straight or branched hydrocarbon chain radical group consisting solely of carbon and hydrogen atoms, containing at least one double bond that can be of either the (E)- or (Z)-configuration, having from two to six carbon atoms, which is attached to the rest of the molecule by a single bond. Examples of C₂-C₆alkenyl include, but are not limited to, prop-1-enyl, allyl (prop-2-enyl), and but-1-enyl.

As used herein, the term “C₂-C₆haloalkenyl” refers to a C₂-C₆alkenyl radical as generally defined above substituted by one or more of the same or different halogen atoms.

As used herein, the term “C₂-C₆alkynyl” refers to a straight or branched hydrocarbon chain radical group consisting solely of carbon and hydrogen atoms, containing at least one triple bond, having from two to six carbon atoms, and which is attached to the rest of the molecule by a single bond. Examples of C₂-C₆alkynyl include, but are not limited to, prop-1-ynyl, propargyl (prop-2-ynyl), and but-1-ynyl.

As used herein, the term “C₂-C₆haloalkynyl” refers to a C₂-C₆alkynyl radical as generally defined above substituted by one or more of the same or different halogen atoms.

As used herein, the term “C₃-C₆cycloalkyl” refers to a stable, monocyclic ring radical which is saturated or partially unsaturated and contains 3 to 6 carbon atoms. C₃-C₄cycloalkyl is to be construed accordingly. Examples of C₃-C₆cycloalkyl include, but are not limited to cyclopropyl, cyclobutyl, cyclopentyl, cyclopenten-1-yl, cyclopenten-3-yl, and cyclohexen-3-yl.

As used herein, the term “C₃-C₆cycloalkylC₁-C₄alkyl” refers to a C₃-C₆cycloalkyl ring as defined above attached to the rest of the molecule by a C₁-C₄alkylene radical as defined above. Examples of C₃-C₆cycloalkylC₁-C₄alkyl include, but are not limited to cyclopropyl-methyl, cyclobutyl-ethyl, and cyclopentyl-methyl.

As used herein, the term “C₃-C₆halocycloalkyl” refers to a C₃-C₆cycloalkyl ring as defined above substituted by one or more of the same or different halogen atoms.

As used herein, the term “C₃-C₆halocycloalkylC₁-C₄alkyl” refers to a C₃-C₆halocycloalkyl radical as defined above attached to the rest of the molecule by a C₁-C₄alkylene radical as defined above.

As used herein, the term “C₁-C₄alkoxyC₁-C₄alkyl” refers to a radical of the formula R_{y}—O—Rₓ— where R_{y} is a C₁-C₄alkyl radical as generally defined above, and Rₓ is a C₁-C₄alkylene radical as generally defined above.

As used herein, the term “C₁-C₄haloalkoxyC₁-C₄alkyl” refers to a radical of the formula R_{y}—O—Rₓ— where R_{y} is a C₁-C₄alkyl radical as generally defined above substituted by one or more of the same or different halogen atoms, and Rₓ is a C₁-C₄alkylene radical as generally defined above.

As used herein, the term “C₁-C₄alkoxyC₁-C₄alkoxy” refers to radical of the formula R_{y}—O—Rₓ—O— where R_{y} is a C₁-C₄alkyl radical as generally defined above, and Rₓ is a C₁-C₄alkylene radical as generally defined above.

As used herein, the term “cyanoC₁-C₄alkyl” refers to a C₁-C₄alkyl radical as generally defined above substituted by one or more cyano groups. CyanoC₁-C₂alkyl should be construed accordingly.

As used herein, the term “cyanoC₁-C₄haloalkyl” refers to a C₁-C₆haloalkyl radical as generally defined above substituted by one or more cyano groups.

As used herein, the term “C₁-C₆alkylsulfanyl” refers to a radical of the formula RₓS— wherein Rₓ is a C₁-C₆alkyl radical as generally defined above.

As used herein, the term “C₁-C₆haloalkylsulfanyl” refers to a C₁-C₆alkylsulfanyl radical as generally defined above substituted by one or more of the same or different halogen atoms.

As used herein, the term “C₁-C₆alkylsulfinyl” refers to a radical of the formula RₓS(O)— wherein Rₓ is a C₁-C₆alkyl radical as generally defined above.

As used herein, the term “C₁-C₆haloalkylsulfinyl” refers to a C₁-C₆alkylsulfinyl radical as generally defined above substituted by one or more of the same or different halogen atoms.

As used herein, the term “C₁-C₆alkylsulfonyl” refers to a radical of the formula RₓS(O)₂— wherein Rₓ is a C₁-C₆alkyl radical as generally defined above.

As used herein, the term “C₁-C₆haloalkylsulfonyl” refers to a C₁-C₆alkylsulfonyl radical as generally defined above substituted by one or more of the same or different halogen atoms.

As used herein, the term “C₁-C₆alkylcarbonyl” refers to a radical of the formula RₓC(O)— where Rₓ is a C₁-C₆alkyl radical as generally defined above.

As used herein, the term “C₁-C₆haloalkylcarbonyl” refers to a C₁-C₆alkylcarbonyl radical as generally defined above substituted by one or more of the same or different halogen atoms.

As used herein, the term “C₁-C₆alkoxycarbonyl” refers to a radical of the formula RₓOC(O)— where Rₓ is a C₁-C₆alkyl radical as generally defined above.

As used herein, the term “C₁-C₆haloalkoxycarbonyl” refers to a C₁-C₆alkoxycarbonyl as generally defined above substituted by one or more of the same or different halogen atoms.

As used herein, the term “C₁-C₆alkylcarbonylamino” refers to a radical of the formula RₓC(O)N(H)— where Rₓ is a C₁-C₆alkyl radical as generally defined above.

As used herein, the term “C₁-C₆haloalkylcarbonylamino” refers to a C₁-C₆alkylcarbonylamino radical as generally defined above substituted by one or more of the same or different halogen atoms.

As used herein, the term “C₃-C₆cycloalkylcarbonylamino” refers to a radical of the formula RₓC(O)N(H)— where Rₓ is a C₃-C₆cycloalkyl radical as generally defined above.

As used herein, the term “C₃-C₆halocycloalkylcarbonylamino” refers to a C₃-C₆cycloalkylcarbonylamino radical as generally defined above substituted by one or more of the same or different halogen atoms.

As used herein, the term “C₁-C₆alkylaminocarbonyl” refers to a radical of the formula RₓNHC(O)— where Rₓ is a C₁-C₆alkyl radical as generally defined above.

As used herein, the term “C₁-C₆haloalkylaminocarbonyl” refers to a C₁-C₆alkylaminocarbonyl radical as generally defined above substituted by one or more of the same or different halogen atoms.

As used herein, the term “C₃-C₆cycloalkylaminocarbonyl” refers to a radical of the formula RₓNHC(O)— where Rₓ is a C₃-C₆cycloalkyl radical as generally defined above.

As used herein, the term “C₃-C₆halocycloalkylaminocarbonyl” refers to a C₃-C₆cycloalkylaminocarbonyl radical as generally defined above substituted by one or more of the same or different halogen atoms.

As used herein, the term “C₃-C₆cycloalkylcarbonyl” refers to a radical of the formula RₓC(O)— where Rₓ is a C₃-C₆cycloalkyl radical as generally defined above.

As used herein, the term “C₃-C₆halocycloalkylcarbonyl” refers to a C₃-C₆cycloalkylcarbonyl radical as generally defined above substituted by one or more of the same or different halogen atoms.

Examples of a 5- to 12-membered heteroaromatic ring system, which can be monocyclic or polycyclic and which comprise 1 to 4 heteroatoms selected from nitrogen, oxygen an sulfur, include pyridyl, pyrimidyl, pyrrolyl, pyrazolyl, furyl, thienyl, imidazolyl, isoxazolyl, oxazolyl, thiazolyl, isothiazolyl, triazolyl, oxadiazolyl, thiadiazolyl, tetrazolyl, pyrazinyl, pyridazinyl, triazinyl, pyranyl, quinazolinyl, isoquinolinyl, indolizinyl, isobenzofuranylnaphthyridinyl, quinoxalinyl, cinnolinyl, phthalazinyl, benzothiazolyl, benzoxazolyl, benzotriazolyl, indazolyl, indolyl, tetrahydroquinolynyl, benzofuryl, benzisofuryl, benzothienyl, benzisothienyl, isoindolyl, naphthyridinyl, benzisothiazolyl, benzisoxazolyl, benzoxazolyl, benzotriazinyl, purinyl, pteridinyl, indolizinyl, phenylpyridyl, and pyridylphenyl.

Examples of a 3- to 12-membered saturated or partially saturated heterocyclic ring system, which can be monocyclic or polycyclic and which comprise 1 to 4 heteroatoms selected from nitrogen, oxygen and sulfur, include dihydropyranyl, tetrahydrofuranyl, tetrahydrothienyl, pyrrolidinyl, isoxazolidinyl, isothiazolidinyl, pyrazolidinyl, oxazolidinyl, thiazolidinyl, imidazolidinyl, oxadiazolidinyl, thiadiazolidinyl, dihydrofuranyl, dihydrothienyl, pyrrolinyl, isoxazolinyl, dihydropyrazolyl, dihydrooxazolyl, piperidinyl, dioxanyl, tetrahydropyranyl, hexahydropyridazinyl, hexahydropyrimidinyl, oxiranyl, and piperazinyl.

Polycyclic as used herein refers to fused cyclic rings, and substituted cyclic rings in which the substituent is another cyclic ring (such as an aryl or heteroaryl ring). Examples of a fused ring are naphthyl, benzisoxazolyl or benzoxazolyl, whereas examples of a substituted ring are biphenyl, 2-phenylpyridyl or 2-pyridylphenyl.

The compounds of formula (I) according to the invention, which have at least one basic centre can form, for example, acid addition salts, for example with strong inorganic acids such as mineral acids, for example perchloric acid, sulfuric acid, nitric acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxy carboxylic acids, for example ascorbic acid, lactic acid, malic acid, tartaric acid or citric acid, or such as benzoic acid, or with organic sulfonic acids, such as C₁-C₄-alkane- or arylsulfonic acids which are unsubstituted or substituted, for example by halogen, for example methane- or p-toluenesulfonic acid. Compounds of formula (I) which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

The presence of one or more possible asymmetric carbon atoms in a compound of Formula (I) means that the compounds may occur in chiral isomeric forms, i.e., enantiomeric or diastereomeric forms. Also, atropisomers may occur as a result of restricted rotation about a single bond. Formula (I) is intended to include all those possible isomeric forms and mixtures thereof. The present invention includes all those possible isomeric forms and mixtures thereof for a compound of Formula (I). Likewise, Formula (I) is intended to include all possible tautomers (including lactam-lactim tautomerism and keto-enol tautomerism) where present. The present invention includes all possible tautomeric forms for a compound of Formula (I).

In each case, the compounds of Formula (I) according to the invention are in free form, in oxidized form as an N-oxide, in covalently hydrated form, or in salt form, e.g., an agronomically usable or agrochemically acceptable salt form. N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991. The compounds of formula (I) according to the invention also include hydrates, which may be formed during salt formation.

The following list provides definitions, including preferred definitions, for substituents W, R¹, R², m, R^{3a}, R^{3b}, R⁴ (ie, Y1, Y2, Y3, Y4, Y5, Y6, Y7), U₁ₐ, U_{1b}, U₂, and U₃ and n with reference to the compounds of Formula (I) of the present invention. For any one of these substituents, any of the definitions given below may be combined with any definition of any other substituent given below or elsewhere in this document.

W is O or S. Preferably, W is O.

R¹ is phenyl or naphthyl, each optionally: (i) mono- or polysubstituted (eg, disubstituted) by a substituent independently selected from U₁ₐ, (ii) mono- or disubstituted by a substituent independently selected from U_{1b}, or (iii) mono- or disubstituted by a substituent independently selected from U₁ₐ and monosubstituted by a substituent selected from U_{1b}; or R¹ is a 5- to 12-membered heteroaromatic ring system or a 3- to 12-membered saturated or partially saturated heterocyclic ring system, wherein the ring system is monocyclic or polycyclic (eg, bicyclic) and comprises 1 to 4 heteroatoms selected from nitrogen, oxygen and sulfur, with the proviso that each ring system cannot contain more than 2 oxygen or sulfur atoms, and wherein each ring system is optionally: (i) mono- or polysubstituted (eg, disubstituted) by a substituent independently selected from U₁ₐ, (ii) mono- or disubstituted by a substituent independently selected from U_{1b}, or (iii) mono- or disubstituted by a substituent independently selected from U₁ₐ and monosubstituted by a substituent selected from U_{1b}.

In some embodiments of the invention, R¹ is phenyl, or a 5- or 6-membered heteroaromatic monocyclic ring system, which ring system comprises 1 or 2 nitrogen atoms or is furanyl or thienyl, wherein each R¹ is optionally:

(i) mono- or polysubstituted (eg, disubstituted) by a substituent independently selected from U₁ₐ,

(ii) mono- or disubstituted by a substituent independently selected from U_{1b}, or

(iii) mono- or disubstituted by a substituent independently selected from U₁ₐ and monosubstituted by a substituent selected from U_{1b}.

In other embodiments of the invention, R¹ is phenyl, pyrazolyl, pyridinyl, pyrimidinyl, pyridazinyl, pyrazinyl, oxazolyl (1,3-oxazaloyl), oxadiazolyl (1,3,4-oxadiazolyl), thiazolyl (1,3-thiazolyl), thiadiazolyl (1,3,4-thiadiazolyl), furanyl or thienyl, wherein each R¹ is optionally:

(i) mono- or polysubstituted (eg, disubstituted) by a substituent independently selected from U₁ₐ,

(ii) mono- or disubstituted by a substituent independently selected from U_{1b}, or

(iii) mono- or disubstituted by a substituent independently selected from U₁ₐ and monosubstituted by a substituent selected from U_{1b}.

In particular, R¹ may be selected from phenyl, pyrazolyl, pyridinyl, pyrimidinyl, pyridazinyl, pyrazinyl, oxazolyl (1,3-oxazaloyl), oxadiazolyl (eg, 1,3,4-oxadiazolyl), thiazolyl (1,3-thiazolyl), thiadiazolyl (eg, 1,3,4-thiadiazolyl), furanyl or thienyl, wherein R¹ is optionally:

(i) mono- or disubstituted by a substituent independently selected from U₁ₐ, wherein U₁ₐ is halogen, C₁-C₄alkyl, C₁-C₄fluoroalkyl, C₁-C₄alkoxy and C₁-C₄fluoroalkoxy, or

(ii) mono-substituted by a substituent selected from U_{1b}, wherein U_{1b} is nitro, cyano, amino, C₃-C₆cycloalkyl, cyanoC₁-C₄alkyl, C₁-C₄alkylcarbonyl, C₁-C₄alkoxycarbonyl, C₁-C₄haloalkylcarbonyl, or phenyl or oxetan-3-yl optionally substituted by a substituent selected from U₂, wherein U₂ is selected from fluoro, chloro, methyl, ethyl, methoxy, ethoxy or trifluoromethyl.

Alternatively, R¹ may be selected from phenyl, pyrazolyl, pyridinyl, pyrimidinyl, pyridazinyl, pyrazinyl, oxazolyl (1,3-oxazaloyl), oxadiazolyl (eg, 1,3,4-oxadiazolyl), thiazolyl (1,3-thiazolyl), thiadiazolyl (eg, 1,3,4-thiadiazolyl), furanyl or thienyl, wherein R¹ is optionally:

(i) mono- or disubstituted by a substituent independently selected from U₁ₐ, wherein U₁ₐ is selected from halogen, C₁-C₄alkyl, difluoromethyl, trifluoromethyl, methoxy, ethoxy and trifluoromethoxy, or

(ii) mono-substituted by a substituent selected from U_{1b}, wherein U_{1b} is nitro, cyano, amino, C₃-C₆cycloalkyl, cyanomethyl, methylcarbonyl, ethylcarbonyl, methoxycarbonyl, ethoxycarbonyl, trifluormethylcarbonyl, or phenyl or oxetan-3-yl.

R¹ may be phenyl, pyrazol-3-yl, pyrazol-4-yl, pyridin-2-yl, pyridin-3-yl, pyridin-4-yl, pyrimidin-2-yl, pyrimidin-4-yl, pyrimidin-5-yl, pyridazin-3-yl, pyrazin-2-yl, oxazol-2-yl, 1,3,4-oxadiazol-2-yl, thiazol-2-yl, thiazol-4-yl, thiazol-5-yl, 1,3,4-thiadiazol-2-yl, furan-2-yl, furan-3-yl, thien-2-yl or thien-3-yl, wherein each R¹ is optionally:

(i) mono- or disubstituted by a substituent independently selected from U₁ₐ, wherein U₁ₐ is halogen, C₁-C₄alkyl, C₁-C₄fluoroalkyl, C₁-C₄alkoxy and C₁-C₄fluoroalkoxy, or

(ii) mono-substituted by a substituent selected from U_{1b}, wherein U_{1b} is nitro, cyano, amino, C₃-C₆cycloalkyl, cyanoC₁-C₄alkyl, C₁-C₄alkylcarbonyl, C₁-C₄alkoxycarbonyl, C₁-C₄haloalkylcarbonyl, or phenyl or oxetan-3-yl optionally substituted by a substituent selected from U₂, wherein U₂ is selected from fluoro, chloro, methyl, ethyl, methoxy, ethoxy or trifluoromethyl.

Alternatively, R¹ may be phenyl, pyrazol-3-yl, pyrazol-4-yl, pyridin-2-yl, pyridin-3-yl, pyridin-4-yl, pyrimidin-2-yl, pyrimidin-4-yl, pyrimidin-5-yl, pyridazin-3-yl, pyrazin-2-yl, oxazol-2-yl, 1,3,4-oxadiazol-2-yl, thiazol-2-yl, thiazol-4-yl, thiazol-5-yl, 1,3,4-thiadiazol-2-yl, furan-2-yl, furan-3-yl, thien-2-yl or thien-3-yl, wherein each R¹ is optionally:

(i) mono- or disubstituted by a substituent independently selected from U₁ₐ, wherein U₁ₐ is selected from halogen, C₁-C₄alkyl, difluoromethyl, trifluoromethyl, methoxy, ethoxy and trifluoromethoxy, or

(ii) mono-substituted by a substituent selected from U_{1b}, wherein U_{1b} is nitro, cyano, amino, C₃-C₆cycloalkyl, cyanomethyl, methylcarbonyl, ethylcarbonyl, methoxycarbonyl, ethoxycarbonyl, trifluormethylcarbonyl, or phenyl or oxetan-3-yl.

In still other embodiments of the invention, R¹ is phenyl, pyrazolyl, pyridinyl, pyrimidinyl, pyridazinyl or pyrazinyl, wherein each R¹ is optionally:

(i) mono- or polysubstituted (eg, disubstituted) by a substituent independently selected from U₁ₐ,

(ii) mono- or disubstituted by a substituent independently selected from U_{1b}, or

(iii) mono- or disubstituted by a substituent independently selected from U₁ₐ and monosubstituted by a substituent selected from U_{1b}.

When R¹ is optionally substituted phenyl, pyrazolyl, pyridinyl, pyrimidinyl, pyridazinyl or pyrazinyl, this may include optionally substituted pyrazol-3-yl, pyrazol-4-yl, pyridin-2-yl, pyridin-3-yl, pyridin-4-yl, pyrimidin-2-yl, pyrimidin-4-yl, pyrimidin-5-yl, pyridazin-3-yl, pyridazin-4-yl, and pyrazin-2-yl.

Preferably, R¹ is phenyl, or a 5- or 6-membered heteroaromatic monocyclic ring system, which ring system comprises 1 or 2 nitrogen atoms, and wherein each R¹ is optionally substituted by:

(i) 1 or 2 substituents independently selected from U₁ₐ, wherein U₁ₐ is halogen, C₁-C₄alkyl, C₁-C₄haloalkyl, C₁-C₄alkoxy and C₁-C₄haloalkoxy, or

(ii) 1 substituent selected from U_{1b}, wherein U_{1b} is cyano or phenyl optionally substituted by 1 substituent selected from U₂, wherein U₂ is fluoro, chloro, methyl, trifluoromethyl, methoxy or cyano.

More preferably, R¹ is phenyl, pyrazolyl, pyridinyl, pyrimidinyl, pyridazinyl or pyrazinyl, wherein each R¹ is optionally substituted by:

(i) 1 or 2 substituents independently selected from U₁ₐ, wherein U₁ₐ is halogen, C₁-C₄alkyl, C₁-C₄haloalkyl, C₁-C₄alkoxy and C₁-C₄haloalkoxy, or

(ii) 1 substituent selected from U_{1b}, wherein U_{1b} is cyano or phenyl optionally substituted by 1 substituent selected from U₂, wherein U₂ is fluoro, chloro, methyl, trifluoromethyl, methoxy or cyano.

Even more preferably, R¹ is phenyl, pyrazolyl, pyridinyl, pyrimidinyl, pyridazinyl or pyrazinyl, wherein each R¹ is optionally substituted by:

(i) 1 or 2 substituents independently selected from U₁ₐ, wherein U₁ₐ is fluoro, chloro, methyl, ethyl, n-propyl, iso-propyl, difluoromethyl, trifluoromethyl, methoxy, ethoxy and trifluoromethoxy, or

(ii) 1 substituent selected from U_{1b}, wherein U_{1b} is cyano or phenyl.

In other embodiments, R¹ is pyrazolyl optionally substituted by 1 or 2 substituents independently selected from U₁ₐ, wherein U₁ₐ is C₁-C₄alkyl or C₁-C₄haloalkyl, or a single substituent selected from U_{1b}, wherein U_{1b} is C₃-C₆cycloalkyl.

Preferably, R¹ is pyrazolyl optionally substituted by 1 or 2 substituents independently selected from U₁ₐ, wherein U₁ₐ is C₁-C₄alkyl or C₁-C₄fluoroalkyl, or a single substituent selected from U_{1b}, wherein U_{1b} is C₃-C₄cycloalkyl.

More preferably, R¹ is pyrazolyl (in particular pyrazol-4-yl), optionally substituted by 1 or 2 substituents independently selected from U₁ₐ, wherein U₁ₐ is methyl, ethyl, n-propyl, iso-propyl, difluoromethyl and trifluoromethyl, or a single substituent from U_{1b}, wherein U_{1b} is cyclopropyl.

U₁ₐ is independently selected from halogen, C₁-C₆alkyl, C₁-C₆haloalkyl, C₁-C₆alkoxy and C₁-C₆haloalkoxy.

U_{1b} is independently selected from nitro, cyano, amino, hydroxyl, —SCN, —CO₂H, C₃-C₆cycloalkyl, C₃-C₆halocycloalkyl, C₃-C₆cycloalkyl-C₁-C₄alkyl, C₃-C₆halocycloalkyl-C₁-C₄alkyl, C₁-C₄alkoxy-C₁-C₄alkyl, C₁-C₄alkoxy-C₁-C₄alkoxy, cyano-C₁-C₄alkyl, cyano-C₁-C₄haloalkyl, C₂-C₆alkenyl, C₂-C₆haloalkenyl, C₂-C₆alkynyl, C₂-C₆haloalkynyl, C₁-C₄haloalkoxy-C₁-C₄alkyl, C₁-C₆alkylsulfanyl, C₁-C₆alkylsulfinyl, C₁-C₆alkylsulfonyl, C₁-C₆haloalkylsulfanyl, C₁-C₆haloalkylsulfinyl, C₁-C₆haloalkylsulfonyl, C₁-C₆alkylcarbonyl, C₁-C₆alkoxycarbonyl, C₁-C₆haloalkylcarbonyl, C₁-C₆haloalkoxycarbonyl, (C₁-C₆alkyl)N(H)—, (C₁-C₆alkyl)₂N—, (C₃-C₆cycloalkyl)N(H)—, (C₃-C₆cycloalkyl)₂N—, C₁-C₆alkylcarbonylamino, C₃-C₆cycloalkylcarbonylamino, C₁-C₆haloalkylcarbonylamino, C₃-C₆halocycloalkylcarbonylamino, C₁-C₆alkylaminocarbonyl, C₃-C₆cycloalkylaminocarbonyl, C₁-C₆haloalkylaminocarbonyl, C₃-C₆halocycloalkylaminocarbonyl, C₃-C₆cycloalkylcarbonyl, C₃-C₆halocycloalkylcarbonyl, —SF₅, —NHS(O)₂ C₁-C₄alkyl, formyl or —C(O)NH₂; or

U_{1b} is phenyl optionally mono- or disubstituted by a group independently selected from U₂; or

U_{1b} is a 5- or 6-membered heteroaromatic ring or a 5- or 6-membered saturated or partially saturated heterocyclic ring, wherein each ring comprises 1 to 4 heteroatoms selected from nitrogen, oxygen and sulfur, with the proviso that each ring cannot contain more than 2 oxygen or sulfur atoms, and wherein each ring is optionally mono- or disubstituted by a group independently selected from U₂.

Preferably, U₁ₐ is selected from halogen, C₁-C₄alkyl, C₁-C₄fluoroalkyl, C₁-C₄alkoxy and C₁-C₄fluoroalkoxy. More preferably, U₁ₐ is selected from halogen, methyl, ethyl, n-propyl, iso-propyl, C₁-C₂fluoroalkyl, methoxy, ethoxy and C₁-C₂fluoroalkoxy. Most preferably, U₁ₐ is selected from is fluoro, chloro, methyl, ethyl, n-propyl, iso-propyl, difluormethyl, trifluoromethyl, methoxy, ethoxy and trifluoromethoxy.

Preferably, U_{1b} is selected from cyano, or phenyl optionally substituted by 1 substituent selected from U₂.

U² is halogen, C₁-C₆alkyl, C₁-C₆haloalkyl, C₁-C₆alkoxy, C₁-C₆haloalkoxy, nitro, cyano, amino, hydroxyl, —SCN, —CO₂H, C₃-C₆cycloalkyl, C₃-C₆halocycloalkyl, C₃-C₆cycloalkyl-C₁-C₄alkyl, C₃-C₆halocycloalkyl-C₁-C₄alkyl, C₁-C₄alkoxy-C₁-C₄alkyl, C₁-C₄alkoxy-C₁-C₄alkoxy, cyano-C₁-C₄alkyl, cyano-C₁-C₄haloalkyl, C₂-C₆alkenyl, C₂-C₆haloalkenyl, C₂-C₆alkynyl, C₂-C₆haloalkynyl, C₁-C₄haloalkoxy-C₁-C₄alkyl, C₁-C₆alkylsulfanyl, C₁-C₆alkylsulfinyl, C₁-C₆alkylsulfonyl, C₁-C₆haloalkylsulfanyl, C₁-C₆haloalkylsulfinyl, C₁-C₆haloalkylsulfonyl, C₁-C₆alkylcarbonyl, C₁-C₆alkoxycarbonyl, C₁-C₆haloalkylcarbonyl, C₁-C₆haloalkoxycarbonyl, —SF₅ or —C(O)NH₂. Preferably, U² is selected from chloro, fluoro, methyl, ethyl, methoxy, cyano and trifluoromethyl.

m is 0, 1 or 2. In some embodiments of the invention, m is 0. In some embodiments of the invention, m is 1 (eg, according to Formula (I-1b)). In some embodiments of the invention, m is 2. Preferably, m is 0 or 1, and more preferably, m is 0.

R² is independently selected from halogen, cyano, amino, hydroxyl, C₁-C₆alkyl, C₁-C₆haloalkyl, C₁-C₆haloalkoxy, C₁-C₆alkoxy, C₂-C₆alkenyl, C₂-C₆haloalkenyl, C₂-C₆alkynyl, C₂-C₆haloalkynyl, C₃-C₆cycloalkyl, C₃-C₆halocycloalkyl, C₁-C₆alkylsulfanyl, C₁-C₆alkylsulfinyl, C₁-C₆alkylsulfonyl, C₁-C₆haloalkylsulfanyl, C₁-C₆haloalkylsulfinyl and C₁-C₆haloalkylsulfonyl.

Preferably, R² is independently selected from halogen, cyano, amino, hydroxyl, C₁-C₄alkyl, C₁-C₄haloalkyl, C₁-C₄haloalkoxy, C₁-C₄alkoxy, C₂-C₄alkenyl, C₂-C₄haloalkenyl, C₂-C₄alkynyl, C₂-C₄haloalkynyl, C₃-C₄cycloalkyl, C₃-C₄halocycloalkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl and C₁-C₄haloalkylsulfonyl. More preferably, R² is independently selected from halogen, cyano, amino, hydroxyl, C₁-C₄alkyl, C₁-C₄fluoroalkyl, C₁-C₄fluoroalkoxy, C₁-C₄alkoxy, C₂-C₄alkenyl, C₂-C₄fluoroalkenyl, C₂-C₄alkynyl, C₂-C₄fluoroalkynyl, C₃-C₄cycloalkyl and C₃-C₄fluorocycloalkyl. Even more preferably, R² is independently selected from fluoro, chloro, methyl, ethyl, trifluoromethyl, trifluoromethoxy, methoxy or ethoxy. Still more preferably, R² is methyl, in particular, when n is 1.

R^{3a} and R^{3b} are independently selected from hydrogen, halogen, C₁-C₄alkyl, C₁-C₄haloalkyl, C₁-C₄alkoxy, C₁-C₄haloalkoxy and cyano. Preferably, R^{3a} is hydrogen and R^{3b} is selected from hydrogen, chloro, fluoro, methyl, ethyl, trifluoromethyl, 2,2-difluoroethyl, 2,2,2-trifluoroethyl, methoxy or ethoxy. More preferably, R^{3a} is hydrogen and R^{3b} is hydrogen or methyl. Most preferably, R^{3a} is hydrogen and R^{3b} is hydrogen.

R₄ is selected from one of Y1 to Y7;

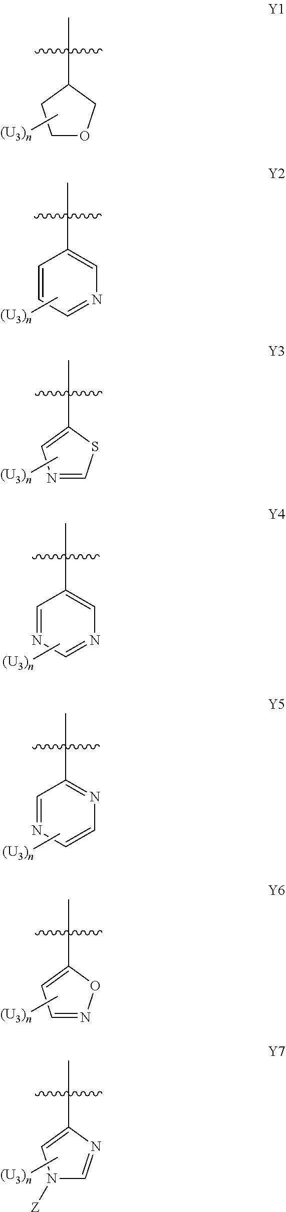

wherein, n is 0, 1, 2, or 3.

Preferably, n is 0 or 1.

Z is hydrogen, C₁-C₄alkyl, C₁-C₄haloalkyl, C₁-C₄alkoxy or C₁-C₄haloalkoxy.

Preferably, Z is hydrogen, methyl, ethyl or trifluoromethyl. More preferably, Z is hydrogen or methyl.

U₃ is independently selected from halogen, cyano, nitro, hydroxyl, amino, C₁-C₄alkyl, C₁-C₄haloalkyl, C₁-C₄alkoxy, C₁-C₄haloalkoxy, C₁-C₄haloalkoxy-C₁-C₄alkyl, C₁-C₄alkoxy-C₁-C₄alkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, formyl, cyclopropyl, C₁-C₆alkylcarbonyl or C₃-C₆cycloalkylcarbonyl.

Preferably, U₃ is independently selected from halogen, cyano, nitro, hydroxyl, amino, C₁-C₄alkyl, C₁-C₄fluoroalkyl, C₁-C₄alkoxy, C₁-C₄fluoroalkoxy, C₁-C₂fluoroalkoxy-C₁-C₂alkyl, C₁-C₂alkoxy-C₁-C₂alkyl, C₁-C₄alkylsulfanyl, C₁-C₄alkylsulfinyl, C₁-C₄alkylsulfonyl, C₁-C₄fluoroalkylsulfanyl, C₁-C₄fluoroalkylsulfinyl, C₁-C₄fluoroalkylsulfonyl, formyl, cyclopropyl, C₁-C₄alkylcarbonyl or C₃-C₆cycloalkylcarbonyl. More preferably, U₃ is independently selected from halogen, cyano, nitro, hydroxyl, amino, methyl, ethyl, trifluoromethyl, methoxy, ethoxy. Most preferably, U₃ is independently selected from fluoro, chloro and trifluoromethyl, and is in particular, chloro.

In some preferred embodiments of the invention, R₄ is selected from one of Y2, Y3 or Y4.

In some preferred embodiments of the invention, R₄ is selected from one of:

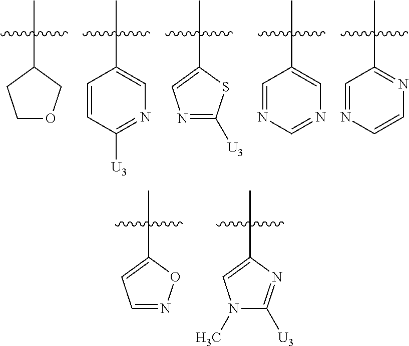

In some preferred embodiments of the invention, R₄ is selected from one of:

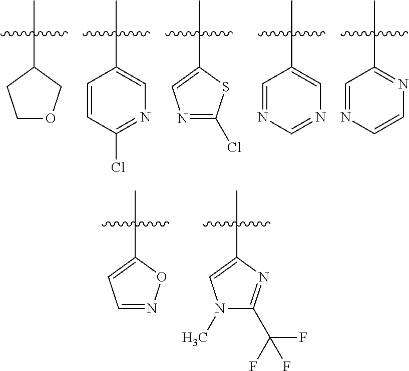

In some preferred embodiments of the invention, R₄ is selected from one of:

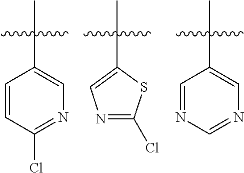

In certain embodiments of the present invention, the compound of Formula (I) is:

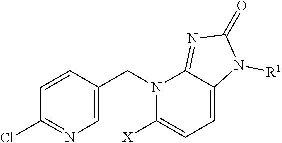

wherein R¹ is defined in accordance with the present invention, and X represents hydrogen or R² as methyl.

In certain embodiments of the present invention, the compound of Formula (I) is:

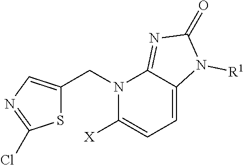

wherein R¹ is defined in accordance with the present invention, and X represents hydrogen or R² as methyl.

In certain embodiments of the present invention, the compound of Formula (I) is:

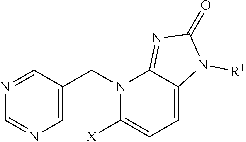

wherein R¹ is defined in accordance with the present invention, and X represents hydrogen or R² as methyl.

Preferably, the compound according to Formula (I) is selected from a compound 1.001 to 1.105 listed in Table 1 (below) or a compound A1 to A102 listed in Table A (below).

In some embodiments, in a compound according to Formula (I) of the invention:
W is O;
R¹ is phenyl, pyrazolyl, pyridinyl, pyrimidinyl, pyridazinyl or pyrazinyl, wherein each R¹ is optionally: (i) mono- or disubstituted by a substituent independently selected from U₁ₐ, (ii) mono- or disubstituted by a substituent independently selected from U_{1b}, or (iii) mono- or disubstituted by a substituent independently selected from U₁ₐ and monosubstituted by a substituent selected from U_{1b};
U₁ₐ is halogen, methyl, ethyl, n-propyl, iso-propyl, C₁-C₂fluoroalkyl, methoxy, ethoxy and C₁-C₂fluoroalkoxy;
U_{1b} is selected from cyano, or phenyl optionally substituted by 1 substituent selected from U₂, wherein U₂ is chloro, fluoro, methyl, ethyl, methoxy, cyano and trifluoromethyl;
R² is methyl;
m is 0 or 1;
R^{3a} and R^{3b} are independently selected from hydrogen and methyl; and
R⁴ is selected from:

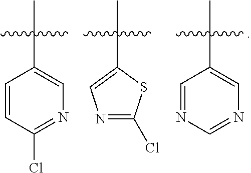

In other embodiments,
W is O;
R¹ is phenyl, pyrazolyl, pyridinyl, pyrimidinyl, pyridazinyl or pyrazinyl, wherein each R¹ is optionally: (i) mono- or disubstituted by a substituent independently selected from U₁ₐ, (ii) mono- or disubstituted by a substituent independently selected from U_{1b}, or (iii) mono- or disubstituted by a substituent independently selected from U₁ₐ and monosubstituted by a substituent selected from U_{1b};
U₁ₐ is fluoro, chloro, methyl, ethyl, n-propyl, iso-propyl, difluormethyl, trifluoromethyl, methoxy, ethoxy and trifluoromethoxy;
U_{1b} is selected from cyano, or phenyl optionally substituted by 1 substituent selected from U₂, wherein U₂ is chloro, fluoro, methyl, ethyl, methoxy, cyano and trifluoromethyl;
m is 0;
R^{3a} and R^{3b} are independently selected from hydrogen and methyl; and
R⁴ is selected from:

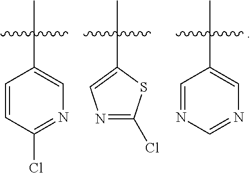

In still other embodiments,
W is O;
R¹ is phenyl, pyrazolyl, pyridinyl, pyrimidinyl, pyridazinyl or pyrazinyl, wherein each R¹ is optionally: (i) mono- or disubstituted by a substituent independently selected from U₁ₐ, (ii) mono- or disubstituted by a substituent independently selected from U_{1b}, or (iii) mono- or disubstituted by a substituent independently selected from U₁ₐ and monosubstituted by a substituent selected from U_{1b};
U₁ₐ is fluoro, chloro, methyl, ethyl, n-propyl, iso-propyl, difluormethyl, trifluoromethyl, methoxy, ethoxy and trifluoromethoxy;
U_{1b} is selected from cyano, or phenyl optionally substituted by 1 substituent selected from U₂, wherein U₂ is chloro, fluoro, methyl, ethyl, methoxy, cyano and trifluoromethyl;
m is 0;
R^{3a} and R^{3b} are hydrogen; and
R⁴ is:

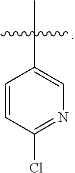 [SMILES: *c1ccc(Cl)nc1]

The compounds of Formula (II) possess the same definitions for m, R², R^{3a}, R^{3b} and R⁴ as for the compounds of Formula (I) and their corresponding preferences.

In the compounds of Formula (II), preferably m is 0 or 1, and more preferably, m is 0.

In the compounds of Formula (II), when m is 1, preferably, R² is C₁-C₄alkyl, and more preferably, methyl.

In the compounds of Formula (II), preferably R^{3a} and R^{3b} are hydrogen.

In the compounds of Formula (II), preferably, R⁴ is selected from one of:

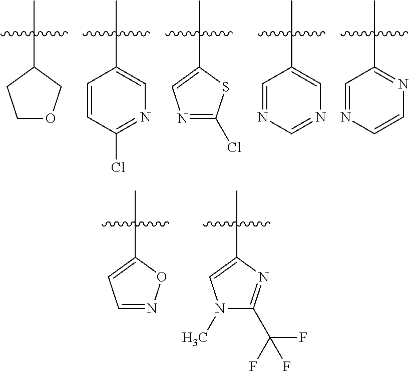

and more preferably,

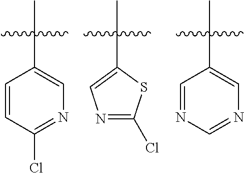

In accordance with the present invention, compounds of Formula (II) may not include the compound where m is 0, R^{3a} and R^{3b} are hydrogen and R⁴ is a 6-chloro-pyridin-3-yl group (ie, 4-[(6-chloro-3-pyridyl)methyl]-1H-imidazo[4,5-b]pyridin-2-one).

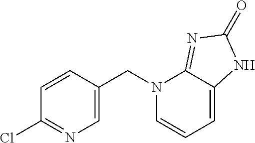 [SMILES: O=c1nc2n(Cc3ccc(Cl)nc3)cccc-2[nH]1]

Compounds of the present invention can be made as shown in the following schemes 1 to 6, in which, unless otherwise stated, the definition of each variable is as defined herein for a compound of formula (I).

Compounds of formula (Ia), wherein R¹, R², R^{3a}, R^{3b}, R⁴ and m are as defined herein, can be readily prepared. A typical example of such a synthesis is shown in Scheme 1 below.

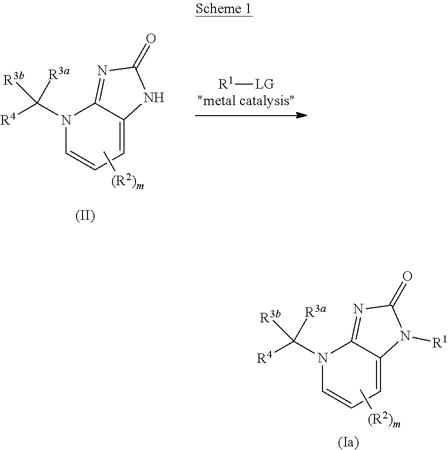

Compounds of formula (Ia) where R¹ is aryl or heteroaryl may be prepared from compounds of formula (II), wherein R², R^{3a}, R^{3b}, R⁴ and m are defined as for formula (I), by metal catalysis coupling with compounds of formula R¹-LG, wherein LG is a leaving group, such as iodide or bromide, in the presence of a base, a copper catalyst and a ligand. This type of coupling, called an Ullmann-type coupling reaction, is well known to those skilled in the art, see for example, Chem. Rev. 2004, 248, pp 2337-2364; Tetrahedron 2011, 67(29), pp 5282-5288; Angew. Chem., Int. Ed. 2003, 42, pp 5400-5449; Synlett 2003, pp 2428-2439; Ind. Eng. Chem. Res. 2005, 44, pp 789-798. The reaction is commonly performed with one to two equivalents of a base, such as potassium phosphate, in the presence of a copper catalyst, such as for example, copper (I) iodide, and under an oxygen-containing atmosphere. The reaction can be run in an inert solvent, such as dioxane or toluene, usually at a temperature between 50 to 150° C. and in the presence or not of an additional ligand, such as for example, diamine ligands (e.g., N,N′-dimethylethylenediamine), dibenzylideneacetone (dba) and 1,10-phenanthroline.

Alternatively, compounds of formula (Ia) where R¹ is aryl or heteroaryl may be prepared from compounds of formula (II), wherein R², R^{3a}, R^{3b}, R⁴ and m are as defined for formula (I) above, by a Buchwald-Hartwig cross coupling, which involves for example, reacting compounds of formula R¹-LG, wherein LG is a leaving group, for example, chloride, bromide or iodide, or an aryl- or alkylsulfonate such as trifluoromethanesulfonate with, for example, compounds of formula (II). The reaction can be catalyzed by a palladium-based catalyst, for example palladium acetate, in the presence of a base, such as cesium carbonate or sodium tert-butoxide, in a solvent or a solvent mixture, such as for example toluene, preferably under an inert atmosphere and in the presence of a chelating phosphine such as 2,2′-bis(diphenylphosphino)-1,1′-binaphthyl (BINAP) or Xantphos. The reaction temperature can preferentially range from ambient temperature to the boiling point of the reaction mixture. Such Buchwald-Hartwig cross coupling reactions are well known to those skilled in the art, many variations are described in literature and have been reviewed, for example, in Strategic Applications of Named Reactions in Organic Synthesis (Kurti, Laszlo; Czako, Barbara; Editors. Ed. ELSEVIER) 2005, p 70 and references cited therein; Modern Tools for the Synthesis of Complex Bioactive Molecules (Chapter 3: Metal-catalyzed C-heteroatom cross-coupling reactions) 2012, p. 77-109.

Alternatively, compounds of formula (Ia) where R¹ is aryl or heteroaryl may be prepared from compounds of formula (II), wherein R², R^{3a}, R^{3b}, R⁴ and m are as defined for formula (I) above, by a Chan-Lam coupling with a compound of formula R¹-LG, wherein LG is a boronic acid function or a boronate or a trifluoroborate group. The reaction may be carried out in the presence of a copper source such as copper(I) iodide (CuI) or copper(II) acetate, optionally in the presence of a suitable base such as pyridine or potassium phosphate. The reaction can be carried out in a suitable solvent such as dichloromethane, dioxane or dimethylsulfoxide at a temperature between 20 and 180° C. under microwave irradiation or not, preferentially under an oxygen atmosphere if the copper salt were used in sub-stoichiometric amounts. See for example Tetrahedron Letters 2015, 56(33), pp 4843-4847; or RSC Advances 2013, 3(29), pp 11472-11475.

Compounds of formula (II), wherein R², R^{3a}, R^{3b}, R⁴ and m are as defined in accordance with the present invention, are generally known or can be readily prepared by those skilled in the art. A typical example of such a synthesis is shown in Scheme 2.

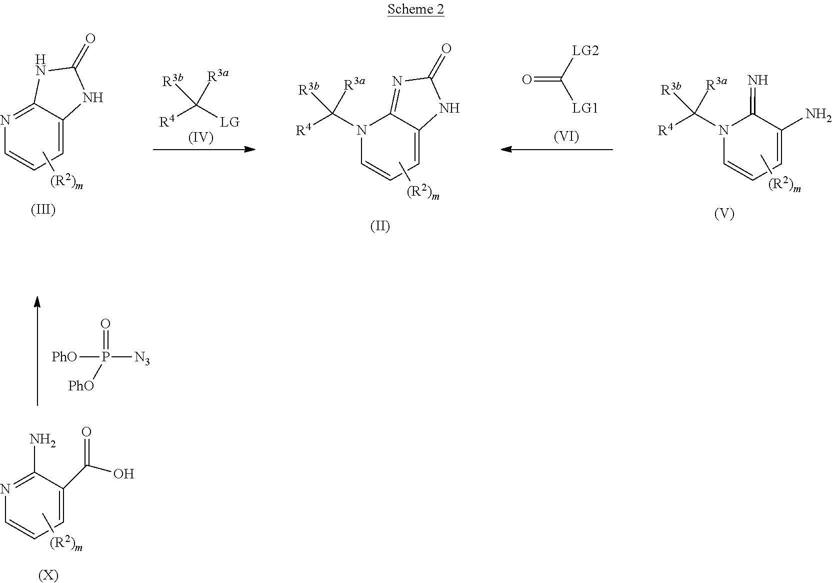

For example, compounds of formula (II) wherein R², R^{3a}, R^{3b}, R⁴ and m are as defined for formula (I), may be prepared by a reaction between a compound of formula (III) and a compound of formula (IV), wherein LG is a leaving group such as, for example, halide, in the presence or not of a suitable base, such as for example sodium carbonate, potassium carbonate or cesium carbonate, or lithium alkoxide, sodium alkoxide or potassium alkoxide, in suitable solvents that may include, for example, acetonitrile, DMF, 1,4-dioxane, and usually upon heating of the reaction mixture at temperatures between room temperature and 200° C., and preferably between 20° C. to the boiling point of the reaction mixture, optionally under microwave heating conditions. A catalyst may be used in this reaction, including sodium iodide or tert-butylammonium iodide.

Alternatively, compounds of formula (II) wherein R², R^{3a}, R^{3b}, R⁴ and m are defined as for formula (I) can be prepared by the reaction of compounds of formula (V) and compounds of formula (VI), wherein LG₁ and LG₂ are independently from each other leaving groups, such as, for example, halide, an aryloxy group or imidazolyl. The reaction can be performed at 0° C. to the boiling point of the solvent and preferably in the presence of a base, which may be, for example, pyridine, Hünig's base, triethylamine or sodium carbonate. Such processes have been described previously, for example, in J Med Chem 2015, 58, p 8066; J. Het. Chem. 2010, 47, p 683; Synth. Comm. 1982, 12, p 213.

Compounds of formula (III) are commercially available or can be readily prepared by those skilled in the art. Many examples can be found in literature, for example, see Mendeleev Communications 2016, 26(1), pp 69-71 and cited references therein; Bulletin of the Chemical Society of Japan 1987, 60(5), pp 1793-9; Journal of Heterocyclic Chemistry 1985, 22(4), pp 1061-4; Heterocycles (2002), 57(12), pp 2335-2343. Alternatively, compounds of formula (III) could be prepared as described in Scheme 2. Compounds of formula (III) wherein R² and m are defined as for formula (I) may be commercially available or may be prepared by a Curtius rearrangement from compounds of formula (X) which are commercially available, wherein R² and m are as described for formula (I), by treatment with, diphenylphosphoryl azide (DPPA), in the presence of a suitable base, such as for example trimethylamine, in suitable solvents that may include, for example, ethanol, tert-butanol, 1,4-dioxane, tetrahydrofuran, usually upon heating at temperatures between room temperature and 200° C., and preferably between 20° C. to the boiling point of the reaction mixture. Such syntheses have been described previously, for example, in Journal of Medicinal Chemistry, 58(20), 8066-8096; 2015. Alternatively, others compounds than compounds of formula (X) could be used such as, for example, analogues of 3-aminopicolinic acid.

Compounds of formula (V), wherein R², R^{3a}, R^{3b}, R⁴ and m are as defined for the present invention, are generally known or can be readily prepared by those skilled in the art. A typical example of such a synthesis is shown in Scheme 3.

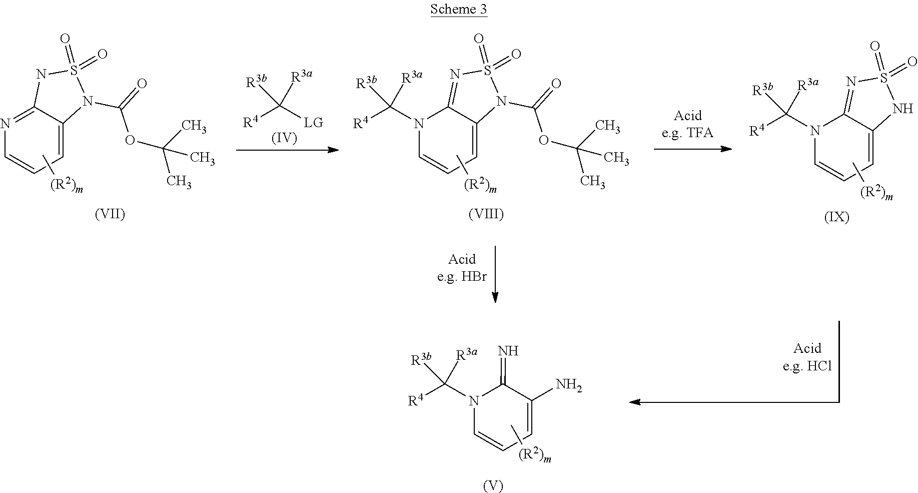

For example, compounds of formula (V) wherein R², R^{3a}, R^{3b}, R⁴ and m are defined as for formula (I) may be prepared by the hydrolysis of compounds of formula (IX) by treatment with a suitable strong acid, such, for example a 37% concentrated aqueous solution of HCl, usually upon heating at temperatures between room temperature and 200° C., and preferably between 20° C. to the boiling point of the reaction mixture, and optionally under microwave heating conditions.

Alternatively, compounds of formula (V) wherein R², R^{3a}, R^{3b}, R⁴ and m are defined as for formula (I) may be prepared by the hydrolysis of compounds of formula (VIII), by treatment with a suitable strong acid, like, for example a 37% concentrated aqueous solution of HBr, usually upon heating at temperatures between room temperature and 200° C., preferably between 20° C. to the boiling point of the reaction mixture, and optionally under microwave heating conditions.

Compounds of formula (IX) wherein R², R^{3a}, R^{3b}, R⁴ and m are defined as for formula (I), may be prepared by hydrolysis of compounds of formula (VIII) by treatment with a suitable acid, such as, for example trifluoroacetic acid (TFA), in suitable solvents that may include, for example, dichloromethane or 1,2-dichloroethane, and usually upon heating at temperatures between room temperature and 200° C., preferably between 20° C. to the boiling point of the reaction mixture, optionally under microwave heating conditions.

Compounds of formula (VIII) wherein R², R^{3a}, R^{3b}, R⁴ and m are as defined for formula (I), may be prepared by reaction between compounds of formula (VII) and compounds of formula (IV), wherein LG is a leaving group such as, for example, halide, in the presence, or not of a suitable base, such as for example sodium carbonate, potassium carbonate or cesium carbonate, or lithium alkoxide, sodium alkoxide or potassium ethanoate, in suitable solvents that may include, for example, acetonitrile, DMF, 1,4-dioxane, and usually upon heating at temperatures between room temperature and 200° C., preferably between 20° C. to the boiling point of the reaction mixture, and optionally under microwave heating conditions. A catalyst may be used in this reaction, including sodium iodide or tert-butylammonium iodide.

Compounds of formula (VII), wherein R₂ and m are as defined for the present invention, are generally known or can be readily prepared by those skilled in the art. A typical example of such synthesis is shown in Scheme 4.

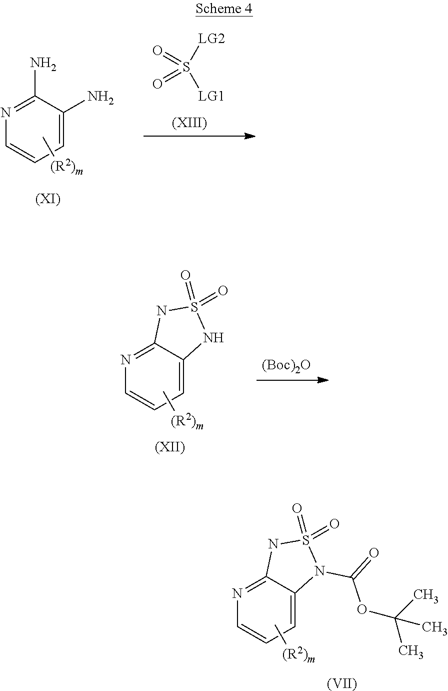

For example, compounds of formula (VII) wherein R² and m are defined as for formula (I) may be prepared from compounds of formula (XII) by treatment with, for example, di-tert-butyl dicarbonate ((Boc)₂O), in a suitable solvent that may include, for example, dichloromethane, 1,2-dichloroethane, tetrahydrofuran or by performing the reaction in molten di-tert-butyl dicarbonate, usually upon heating at temperatures between room temperature and 200° C., preferably between 20° C. to the boiling point of the reaction mixture. Alternative protecting group could be used, such as for example, benzyl. Compounds of formula (XII) may be prepared by the reaction of compounds of formula (XI) and compounds of formula (XIII), wherein LG₁ and LG₂ are independently from each other leaving groups, like, for example amino, halide or imidazole. The reaction can be performed at 0° C. to the boiling point of the solvent and preferably in the presence of a base, which could be, for example, pyridine, Hünig's base, triethylamine or sodium carbonate. Such processes have been described previously, Eur. J. Med. Chem. 2016, 113, p 102. Compounds of formula (XI) are commercially available or can be readily prepared by those skilled in the art.

Alternatively, the sequence to prepare the compounds of formula (Ia), wherein R¹, R², R^{3a}, R^{3b} and R⁴ are as described under formula (I) above, from compounds of formula (XIV) may involve:

(i) halogen displacement of a compound of formula (XIV) wherein LG is a leaving group such as fluoride or bromide, with a compound of formula (XVIII), to form a compound of formula (XV). This reaction may be done by nucleophilic displacement of LG (eg, fluoro) or by metal catalysis such as via Buchwald-Hartwig cross-coupling. The nucleophilic displacement of LG takes place under basic conditions, such as with potassium carbonate in a solvent such as DMF, at from ambient temperature to the boiling point of the reaction mixture. The Buchwald-Hartwig cross-coupling reaction can be catalyzed by a palladium-based catalyst (eg, palladium acetate) in the presence of a base (eg, cesium carbonate or sodium tert-butoxide) in a solvent (eg, toluene) or solvent mixture and preferably under inert an atmosphere in the presence of a chelating phosphine, such as BINAP or Xantphos. The reaction temperature can preferentially range from ambient temperature to the boiling point of the reaction mixture. Such Buchwald-Hartwig cross-coupling reactions are well-known to those skilled in the art. Many variations are described in literature and have been reviewed, eg, in Strategic Applications of Named Reactions in Organic Synthesis (Kurti, Laszlo; Czako, Barbara; Editors. Ed. ELSEVIER) 2005, p 70 and references cited therein; Modern Tools for the Synthesis of Complex Bioactive Molecules (Chapter 3: Metal-catalyzed C-heteroatom cross-coupling reactions) 2012, p. 77-109. Alternatively, the cross-coupling reaction can be catalyzed by copper catalyst and a ligand. This type of coupling is called an Ullmann-type coupling reaction and is described in scheme 1.

(ii) reduction of the nitro derivative (XV) to generate the compound of formula (XVI) via classical conditions, see for example, Synthetic Organic Methodology: Comprehensive Organic Transformations. A Guide to Functional Group Preparations, Larock, R. C. 1989 p. 411. For references describing examples of steps (i) and/or (ii) see WO 2011/123751, WO 2017/178819 or European Journal of Organic Chemistry (22), 3753-3764, S3753/1-S3753/35; 2009;

(iii) Reaction of the compound of formula (XVI) with a compound of formula (VI) by an analogous process to the method described in Scheme 2 to yield a compound of formula (XVII); and

(iv) alkylation of the pyridinyl fragment of the compound of formula (XVII) with a compound of formula (IV) by analogy with the method described in scheme 2 to yield the compound of formula (Ia).

See scheme 5.

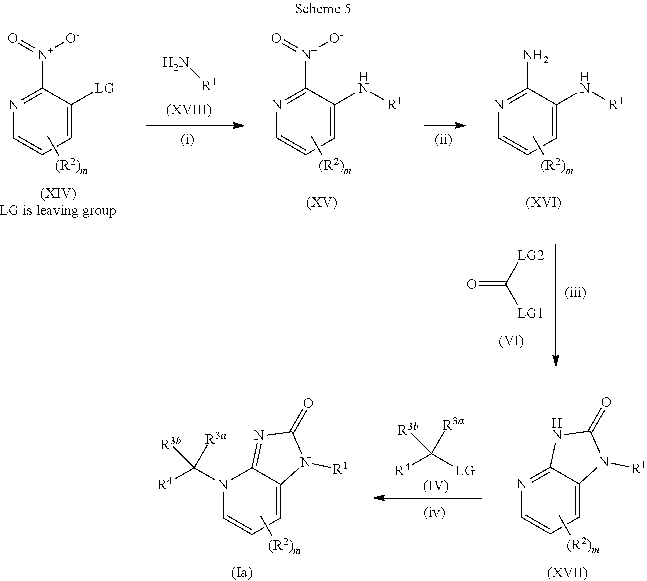

Alternatively, the sequence to prepare the compounds of formula (Ia), wherein R¹, R², R^{3a}, R^{3b} and R⁴ are as described under formula (I) above, from compounds of formula (XVI) may involve:

(i) alkylation of the pyridinyl fragment of the compound of formula (XVI) with a compound of formula (IV) by analogy with the method described in scheme 2 to yield the compound of formula (XIX).

(ii) Reaction of the compound of formula (XIX) wherein R¹, R², m, R^{3a}, R^{3b} and R⁴ are as described for formula (I) with at least two equivalents of a compound of formula X—CN (XX), wherein X is a halogen, such as bromine, to give the compound of formula (XXI). The reaction occurs in the presence or not of a base (such as triethylamine or sodium hydride), in the presence or not of a catalysis, such as 4-dimethylaminopyridine, in an appropriate solvent (eg, N,N-dimethylformamide, N,N-dimethylacetamide or acetonitrile) or in the absence of solvent, at temperatures between −78° C. and 150° C., and preferably between 0° C. and 150° C. Analogues reactions are well known to those skilled in the art and similar conditions could be used, for example, see EP 0 427 526; European Journal of Medicinal Chemistry (1993), 28(7-8), 633-6 or WO 2017/005673.

(iii) Hydrolysis of the compound of formula (XXI), wherein R¹, R², R^{3a}, R^{3b} and R⁴ are as described under formula (I) above under acidic conditions, such as hydrogen chloride in solvent such as water or a mixture of water and alcohol (e.g ethanol) at temperature between −20° C. to reflux, to give a compound of formula (Ia). See scheme 6.

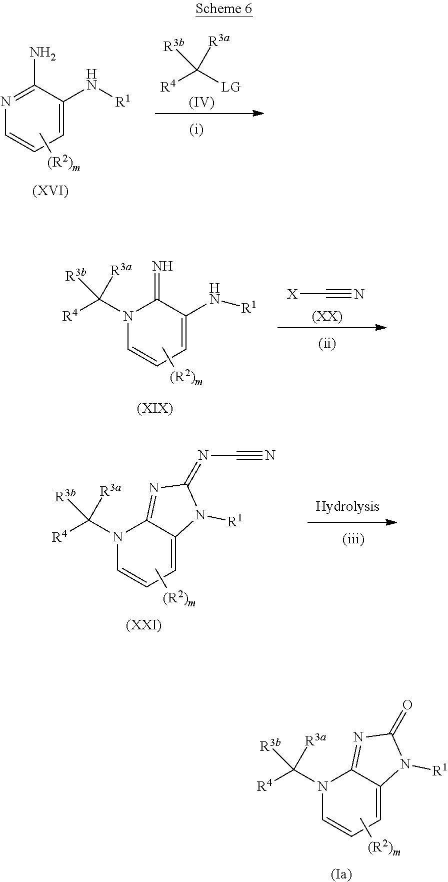

In accordance with the reactions described in any of Schemes 1 to 6, examples of suitable bases may include alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylamnnoniunn hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

Reactions are advantageously carried out in a temperature range from approximately −80° C. to approximately 140° C., preferably from approximately −30° C. to approximately 100° C., in many cases in the range between ambient temperature and approximately 80° C.

A compound of formula (I) can be converted in a manner known per se into another compound of formula (I) by replacing one or more substituents of the starting compound of formula (I) in the customary manner by (an)other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula (I) can be prepared in a manner known per se. Thus, for example, acid addition salts of compounds of formula (I) are obtained by treatment with a suitable acid or a suitable ion exchanger reagent and salts with bases are obtained by treatment with a suitable base or with a suitable ion exchanger reagent.

Salts of compounds of formula (I) can be converted in the customary manner into the free compounds (I), acid addition salts, for example, by treatment with a suitable basic compound or with a suitable ion exchanger reagent and salts with bases, for example, by treatment with a suitable acid or with a suitable ion exchanger reagent.

Salts of compounds of formula (I) can be converted in a manner known per se into other salts of compounds of formula (I), acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula (I), which have salt-forming properties, can be obtained in free form or in the form of salts.

The compounds of formula (I) and, where appropriate, the tautomer's thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule, the invention relates to the pure isomers and also to all isomer mixtures which are possible and is to be understood in each case in this sense hereinabove and herein below, even when stereochemical details are not mentioned specifically in each case.

Diastereomeric mixtures or racemic mixtures of compounds of formula (I), in free form or in salt form, which can be obtained depending on which starting materials and procedures have been chosen can be separated in a known manner into the pure diastereomers or racemates on the basis of the physicochemical differences of the components, for example by fractional crystallization, distillation and/or chromatography.

Enantiomeric mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula (I) and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds of formula (I) according to the invention are preventively and/or curatively valuable active ingredients in the field of pest control, even at low rates of application, which have a very favorable biocidal spectrum and may be well-tolerated by warm-blooded species, fish and plants. The compounds of formula (I) may have a beneficial safety profile towards non-target species, such as bees, and accordingly a good toxicity profile. The active ingredients according to the invention may act against all or individual developmental stages of normally sensitive, but also resistant pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate.

Examples of the above-mentioned pests are:

from the order Acarina, for example,

Acalitus spp., Aculus spp., Acaricalus spp., Aceria spp., Acarus siro, Amblyomma spp., Argas spp., Boophilus spp., Brevipalpus spp., Bryobia spp., Calipitrimerus spp., Chorioptes spp., Dermanyssus gallinae, Dermatophagoides spp., Eotetranychus spp., Eriophyes spp., Hemitarsonemus spp., Hyalomma spp., Ixodes spp., Olygonychus spp., Ornithodoros spp., Polyphagotarsone latus, Panonychus spp., Phyllocoptruta oleivora, Phytonemus spp., Polyphagotarsonemus spp., Psoroptes spp., Rhipicephalus spp., Rhizoglyphus spp., Sarcoptes spp., Steneotarsonemus spp., Tarsonemus spp. and Tetranychus spp.,

from the order Anoplura, for example,

Haematopinus spp., Linognathus spp., Pediculus spp., Pemphigus spp. and Phylloxera spp.,

from the order Coleoptera, for example,

Agriotes spp., Amphimallon majale, Anomala orientalis, Anthonomus spp., Aphodius spp., Astylus atromaculatus, Ataenius spp., Atomaria linearis, Chaetocnema tibialis, Cerotoma spp., Conoderus spp., Cosmopolites spp., Cotinis nitida, Curculio spp., Cyclocephala spp., Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp., Maecolaspis spp., Maladera castanea, Megascelis spp., Melighetes aeneus, Melolontha spp., Myochrous armatus, Orycaephilus spp., Otiorhynchus spp., Phyllophaga spp., Phlyctinus spp., Popillia spp., Psylliodes spp., Rhyssomatus aubtilis, Rhizopertha spp., Scarabeidae, Sitophilus spp., Sitotroga spp., Somaticus spp., Sphenophorus spp., Sternechus subsignatus, Tenebrio spp., Tribolium spp. and Trogoderma spp.,

from the order Diptera, for example,

Aedes spp., Anopheles spp., Antherigona soccata, Bactrocea oleae, Bibio hortulanus, Bradysia spp., Calliphora erythrocephala, Ceratitis spp., Chrysomyia spp., Culex spp., Cuterebra spp., Dacus spp., Delia spp., Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp., Rivelia quadrifasciata, Scatella spp., Sciara spp., Stomoxys spp., Tabanus spp., Tannia spp. and Tipula spp.,

from the order Hemiptera, for example,

Acanthocoris scabrator, Acrosternum spp., Adelphocoris lineolatus, Amblypelta nitida, Bathycoelia thalassina, Blissus spp., Cimex spp., Clavigralla tomentosicollis, Creontiades spp., Distantiella theobroma, Dichelops furcatus, Dysdercus spp., Edessa spp., Euchistus spp., Eurydema pulchrum, Eurygaster spp., Euschistus spp. (stinkbugs), Halyomorpha halys, Horcias nobilellus, Leptocorisa spp., Lygus spp., Margarodes spp., Murgantia histrionic, Neomegalotomus spp., Nesidiocoris tenuis, Nezara spp., Nysius simulans, Oebalus insularis, Piesma spp., Piezodorus spp., Rhodnius spp., Sahlbergella singularis, Scaptocoris castanea, Scotinophara spp., Thyanta spp., Triatoma spp., Vatiga illudens,

Acyrthosium pisum, Adalges spp., Agalliana ensigera, Agonoscena targionii, Aleurodicus spp., Aleurocanthus spp., Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp., Brachycaudus spp., Brevicoryne brassicae, Cacopsylla spp., Cavariella aegopodii Scop., Ceroplaster spp., Chrysomphalus aonidium, Chrysomphalus dictyospermi, Cicadella spp., Cofana spectra, Cryptomyzus spp., Cicadulina spp., Coccus hesperidum, Dalbulus maidis, Dialeurodes spp., Diaphorina citri, Diuraphis noxia, Dysaphis spp., Empoasca spp., Eriosoma larigerum, Erythroneura spp., Gascardia spp., Glycaspis brimblecombei, Hyadaphis pseudobrassicae, Hyalopterus spp., Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp., Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp., Phorodon humuli, Phylloxera spp., Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp., Trialeurodes spp., Tridiscus sporoboli, Trionymus spp., Trioza erytreae, Unaspis citri, Zygina flammigera, Zyginidia scutellaris,

from the order Hymenoptera, for example,

Acromyrmex, Arge spp., Atta spp., Cephus spp., Diprion spp., Diprionidae, Gilpinia polytoma, Hoplo-campa spp., Lasius spp., Monomorium pharaonis, Neodiprion spp., Pogonomyrmex spp., Slenopsis invicta, Solenopsis spp. and Vespa spp.,

from the order Isoptera, for example,

Coptotermes spp., Corniternes cumulans, Incisitermes spp., Macrotermes spp., Mastotermes spp., Microtermes spp., Reticulitermes spp., Solenopsis geminate

from the order Lepidoptera, for example,

Acleris spp., Adoxophyes spp., Aegeria spp., Agrotis spp., Alabama argillaceae, Amylois spp., Anticarsia gemmatalis, Archips spp., Argyresthia spp., Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp., Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp., Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp., Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp., Noctua spp., Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp., Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.,

from the order Mallophaga, for example,

Damalinea spp. and Trichodectes spp.,

from the order Orthoptera, for example,

Blatta spp., Blattella spp., Gryllotalpa spp., Leucophaea maderae, Locusta spp., Neocurtilla hexadactyla, Periplaneta spp., Scapteriscus spp., and Schistocerca spp.,

from the order Psocoptera, for example,

Liposcelis spp.,

from the order Siphonaptera, for example,

Ceratophyllus spp., Ctenocephalides spp. and Xenopsylla cheopis,

from the order Thysanoptera, for example,

Calliothrips phaseoli, Frankliniella spp., Heliothrips spp., Hercinothrips spp., Parthenothrips spp., Scirtothrips aurantii, Sericothrips variabilis, Taeniothrips spp., Thrips spp.,

from the order Thysanura, for example, Lepisma saccharina.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum, beet, such as sugar or fodder beet, fruit, for example pomaceous fruit, stone fruit or soft fruit, such as apples, pears, plums, peaches, almonds, cherries or berries, for example strawberries, raspberries or blackberries, leguminous crops, such as beans, lentils, peas or soya, oil crops, such as oilseed rape, mustard, poppies, olives, sunflowers, coconut, castor, cocoa or ground nuts, cucurbits, such as pumpkins, cucumbers or melons, fibre plants, such as cotton, flax, hemp or jute, citrus fruit, such as oranges, lemons, grapefruit or tangerines, vegetables, such as spinach, lettuce, asparagus, cabbages, carrots, onions, tomatoes, potatoes or bell peppers, Lauraceae, such as avocado, Cinnamonium or camphor, and also tobacco, nuts, coffee, eggplants, sugarcane, tea, pepper, grapevines, hops, the plantain family, latex plants and ornamentals.

The active ingredients according to the invention may especially be suitable for controlling Aphis craccivora, Diabrotica balteata, Thrips tabaci, Euschistus heros, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species, cyst-forming nematodes, Globodera rostochiensis and other Globodera species, Heterodera avenae, Heterodera glycines, Heterodera schachtii, Heterodera trifolii, and other Heterodera species, Seed gall nematodes, Anguina species, Stem and foliar nematodes, Aphelenchoides species, Sting nematodes, Belonolaimus longicaudatus and other Belonolaimus species, Pine nematodes, Bursaphelenchus xylophilus and other Bursaphelenchus species, Ring nematodes, Criconema species, Criconemella species, Criconemoides species, Mesocriconema species, Stem and bulb nematodes, Ditylenchus destructor, Ditylenchus dipsaci and other Ditylenchus species, Awl nematodes, Dolichodorus species, Spiral nematodes, Heliocotylenchus multicinctus and other Helicotylenchus species, Sheath and sheathoid nematodes, Hemicycliophora species and Hemicriconemoides species, Hirshmanniella species, Lance nematodes, Hoploaimus species, false rootknot nematodes, Nacobbus species, Needle nematodes, Longidorus elongatus and other Longidorus species, Pin nematodes, Pratylenchus species, Lesion nematodes, Pratylenchus neglectus, Pratylenchus penetrans, Pratylenchus curvitatus, Pratylenchus goodeyi and other Pratylenchus species, Burrowing nematodes, Radopholus similis and other Radopholus species, Reniform nematodes, Rotylenchus robustus, Rotylenchus reniformis and other Rotylenchus species, Scutellonema species, Stubby root nematodes, Trichodorus primitivus and other Trichodorus species, Paratrichodorus species, Stunt nematodes, Tylenchorhynchus claytoni, Tylenchorhynchus dubius and other Tylenchorhynchus species, Citrus nematodes, Tylenchulus species, Dagger nematodes, Xiphinema species, and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae, Arion (A. ater, A. circumscriptus, A. hortensis, A. rufus), Bradybaenidae (Bradybaena fruticum), Cepaea (C. hortensis, C. Nemoralis), ochlodina, Deroceras (D. agrestis, D. empiricorum, D. laeve, D. reticulatum), Discus (D. rotundatus), Euomphalia, Galba (G. trunculata), Helicelia (H. itala, H. obvia), Helicidae Helicigona arbustorum), Helicodiscus, Helix (H. aperta), Limax (L. cinereoniger, L. flavus, L. marginatus, L. maximus, L. tenellus), Lymnaea, Milax (M. gagates, M. marginatus, M. sowerbyi), Opeas, Pomacea (P. canaticulata), Vallonia and Zanitoides.

Compounds according to Formula (I) may find utility in controlling resistant populations of insects previously sensitive to the neonicotinoid class of pesticidal (insecticidal) agents (the “neonicotinoids”). Accordingly, the present invention may relate to a method of controlling insects which are resistant to a neonicotinoid insecticide comprising applying a compound of Formula (I) (eg, a single compound selected from compounds 1.001 to 1.105 listed in Table 1 (below) or a compound A1 to A102 listed in Table A (below) to a neonicotinoid-resistant insect. Likewise, the present invention may relate to the use of a compound of Formula (I) (eg, a single compound selected from compounds 1.001 to 1.105 listed in Table 1 (below) or a compound A1 to A102 listed in Table A (below) as an insecticide against neonicotinoid-resistant insects. Such neonicotinoid-resistant insects may include insects from the order Lepidoptera or Hemiptera, in particular from the family Aphididae.

The neonicotinoids represent a well-known class of insecticides introduced to the market since the commercialization of pyrethroids (Nauen & Denholm, 2005: Archives of Insect Biochemistry and Physiology 58:200-215) and are extremely valuable insect control agents, not least because they had exhibited little or no cross-resistance to older insecticide classes, which suffer markedly from resistance problems. However, reports of insect resistance to the neonicotinoid class of insecticides are on the increase.

The increase in resistance of such insects to neonicotinoid insecticides thus poses a significant threat to the cultivation of a number of commercially important crops, fruits and vegetables, and there is thus a need to find alternative insecticides capable of controlling neonicotinoid resistant insects (i.e. to find insecticides that do not exhibit any cross-resistance with the neonicotinoid class).

Resistance may be defined as “a heritable change in the sensitivity of a pest population that is reflected in the repeated failure of a product containing an insecticidal active ingredient to achieve the expected level of control when used according to the label recommendation for that pest species” (IRAC). Cross-resistance occurs when resistance to one insecticide confers resistance to another insecticide via the same biochemical mechanism. This can happen within insecticide chemical groups or between insecticide chemical groups. Cross-resistance may occur even if the resistant insect has never been exposed to one of the chemical classes of insecticide.

Two of the major mechanisms for neonicotinoid resistance include:—
(i) Target site resistance, whereby resistance is associated with replacement of one or more amino acids in the insecticide target protein (i.e. the nicotinic acetylcholine receptor); and
(ii) Metabolic resistance, such as enhanced oxidative detoxification of neonicotinoids due to overexpression of monooxygenases;

For general review on insect resistance to neonicotinoid insecticides see, for example, Pesticide Biochemistry and Physiology (2015), 121, 78-87 or Advances in Experimental Medicine and Biology (2010), 683 (Insect Nicotinic Acetylcholine Receptors), 75-83.

The cytochrome P450 monooxygenases are an important metabolic system involved in the detoxification/activation of xenobiotics. As such, P450 monooxygenases play an important role in insecticide resistance. P450 monooxygenases have such a phenomenal array of metabolisable substrates because of the presence of numerous P450s (60-111) in each species, as well as the broad substrate specificity of some P450s. Studies of monooxygenase-mediated resistance have indicated that resistance can be due to increased expression of one P450 (via increased transcription) involved in detoxification of the insecticide and might also be due to a change in the structural gene itself. As such, metabolic cross-resistance mechanisms affect not only insecticides from the given class (e.g. neonicotinoids) but also seemingly unrelated insecticides. For example, cross-resistance relationships between the neonicotinoids and pymetrozine in Bemisia tabaci have been reported by Gorman et al (Pest Management Science 2010, p. 1186-1190). Or for example, for evidence on detoxification via P450, see, for example, Harrop, Thomas W R and al. Pest Management Science (2018), 74(7), p 1616-1622 and cited references.

Target site resistance of nicotinoids is well studied and it has been shown that modification of the active site of nicotinic acetylcholine receptor confers the resistance to nicotinoids. For example, see Bass et al BMC Neuroscience (2011), 12, p 51, Pest Management Science (2018), 74(6), 1297-1301,

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae, or insecticidal proteins from Bacillus thuringiensis, such as 5-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A, or insecticidal proteins of bacteria colonising nematodes, for example Photorhabdus spp. or Xenorhabdus spp., such as Photorhabdus luminescens, Xenorhabdus nematophilus, toxins produced by animals, such as scorpion toxins, arachnid toxins, wasp toxins and other insect-specific neurotoxins, toxins produced by fungi, such as Streptomycetes toxins, plant lectins, such as pea lectins, barley lectins or snowdrop lectins, agglutinins, proteinase inhibitors, such as trypsin inhibitors, serine protease inhibitors, patatin, cystatin, papain inhibitors, ribosome-inactivating proteins (RIP), such as ricin, maize-RIP, abrin, luffin, saporin or bryodin, steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 5-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cry1-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin), YieldGard Rootworm® (maize variety that expresses a Cry3Bb1 toxin), YieldGard Plus® (maize variety that expresses a Cry1Ab and a Cry3Bb1 toxin), Starlink® (maize variety that expresses a Cry9C toxin), Herculex I® (maize variety that expresses a Cry1Fa2 toxin and the enzyme phosphinothricine N-acetyltransferase (PAT) to achieve tolerance to the herbicide glufosinate ammonium), NuCOTN 33B® (cotton variety that expresses a Cry1Ac toxin), Bollgard I® (cotton variety that expresses a Cry1Ac toxin), Bollgard II® (cotton variety that expresses a Cry1Ac and a Cry2Ab toxin), VipCot® (cotton variety that expresses a Vip3A and a Cry1Ab toxin), NewLeaf® (potato variety that expresses a Cry3A toxin), NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.

2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.

3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.

5. IPC 531 Cotton from Monsanto Europe S.A. 270-272 Avenue de Tervuren, B-1150 Brussels, Belgium, registration number C/ES/96/02.

6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins, stilbene synthases, bibenzyl synthases, chitinases, glucanases, the so-called “pathogenesis-related proteins” (PRPs, see e.g. EP-A-0 392 225), antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store ambients and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors, see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents. A further object of the invention is therefore a substrate selected from nonwoven and fabric material comprising a composition which contains a compound of formula I.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 03/034823, U.S. Pat. No. 5,631,072, WO 2005/64072, WO 2006/128870, EP 1724392, WO 2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following Table:

Examples of Exotic Woodborers of Economic Importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus | Apple, Pear, Cranberry, |
| | vittaticolllis | Serviceberry, Hawthorn |
| | Chrysobothris | Apple, Apricot, Beech, Boxelder, |
| | femorata | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania | Basswood, Beech, Maple, Oak, |
| | campestris | Sycamore, Willow, Yellow-poplar |
| Cerambycidae | Goes | Beech, Elm, Nuttall, Willow, Black |
| | pulverulentus | oak, Cherrybark oak, Water oak, |
| | | Sycamore |
| | Goes tigrinus | Oak |
| | Neoclytus | Ash, Hickory, Oak, Walnut, Birch, |
| | acuminatus | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes | Fig, Alder, Mulberry, Willow, |
| | trilineatus | Netleaf hackberry |
| | Oberea | Sumac, Apple, Peach, Plum, |
| | ocellata | Pear, Currant, Blackberry |
| | Oberea | Dogwood, Viburnum, Elm, |
| | tripunctata | Sourwood, Blueberry, |
| | | Rhododendron, Azalea, Laurel, |
| | | Poplar, Willow, Mulberry |
| | Oncideres | Hickory, Pecan, Persimmon, Elm, |
| | cingulata | Sourwood, Basswood, Honeylocust, |
| | | Dogwood, Eucalyptus, Oak, |
| | | Hackberry, Maple, Fruit trees |
| | Saperda | Poplar |
| | calcarata | |
| | Strophiona | Chestnut, Oak, Hickory, Walnut, |
| | nitens | Beech, Maple |
| Scolytidae | Corthylus | Maple, Oak, Yellow-poplar, |
| | columbianus | Beech, Boxelder, Sycamore, Birch, |
| | | Basswood, Chestnut, Elm |
| | Dendroctonus | Pine |
| | frontalis | |
| | Dryocoetes | Birch, Sweetgum, Wild cherry, |
| | betulae | Beech, Pear |
| | Monarthrum | Oak, Maple, Birch, Chestnut, |
| | fasciatum | Sweetgum, Blackgum, Poplar, |
| | | Hickory, Mimosa, Apple, Peach, Pine |
| | Phloeotribus | Peach, Cherry, Plum, Black |
| | liminaris | cherry, Elm, Mulberry, Mountain-ash |
| | Pseudo- | Oak, American beech, Black |
| | pityophthorus | cherry, Chickasaw plum, Chestnut, |
| | pruinosus | Maple, Hickory, Hornbeam, |
| | | Hophornbeam |
| Sesiidae | Paranthrene | Oak, American chestnut |
| | simulans | |
| | Sannina | Persimmon |
| | uroceriformis | |
| | Synanthedon | Peach, Plum, Nectarine, Cherry, |
| | exitiosa | Apricot, Almond, Black cherry |
| | Synanthedon | Peach, Plum, Cherry, Beach, |
| | pictipes | Black Cherry |
| | Synanthedon | Tupelo |
| | rubrofascia | |
| | Synanthedon | Dogwood, Pecan, Hickory, Oak, |
| | scitula | Chestnut, Beech, Birch, Black cherry, |
| | | Elm, Mountain-ash, Viburnum, |
| | | Willow, Apple, Loquat, Ninebark, |
| | | Bayberry |
| | Vitacea | Grape |
| | polistiformis | |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged, Scapteriscus spp., Gryllotalpa africana) and leatherjackets (European crane fly, Tipula spp).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:
Of the order Anoplurida: Haematopinus spp., Linognathus spp., Pediculus spp. and Phtirus spp., Solenopotes spp.,
Of the order Mallophagida: Trimenopon spp., Menopon spp., Trinoton spp., Bovicola spp., Werneckiella spp., Lepikentron spp., Damalina spp., Trichodectes spp. and Felicola spp.,
Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.,
Of the order Siphonapterida, for example Pulex spp., Ctenocephalides spp., Xenopsylla spp., Ceratophyllus spp.,
Of the order Heteropterida, for example Cimex spp., Triatoma spp., Rhodnius spp., Panstrongylus spp.,
Of the order Blattarida, for example Blatta orientalis, Periplaneta americana, Blatt elagermanica and Supella spp.,
Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.,
Of the orders Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec, and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds according to the invention can be used as pesticidal agents in unmodified form, but they are generally formulated into compositions in various ways using formulation adjuvants or addditives, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate, salts of alkylarylsulfonates, such as calcium dodecylbenzenesulfonate, alkylphenol/alkylene oxide addition products, such as nonylphenol ethoxylate, alcohol/alkylene oxide addition products, such as tridecylalcohol ethoxylate, soaps, such as sodium stearate, salts of alkylnaphthalenesulfonates, such as sodium dibutylnaphthalenesulfonate, dialkyl esters of sulfosuccinate salts, such as sodium di(2-ethylhexyl)sulfosuccinate, sorbitol esters, such as sorbitol oleate, quaternary amines, such as lauryltrimethylammonium chloride, polyethylene glycol esters of fatty acids, such as polyethylene glycol stearate, block copolymers of ethylene oxide and propylene oxide, and salts of mono- and di-alkylphosphate esters, and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

Emulsifiable Concentrates:

active ingredient: 1 to 95%, preferably 60 to 90%

surface-active agent: 1 to 30%, preferably 5 to 20%

liquid carrier: 1 to 80%, preferably 1 to 35%

Dusts:

active ingredient: 0.1 to 10%, preferably 0.1 to 5%

solid carrier: 99.9 to 90%, preferably 99.9 to 99%

Suspension Concentrates:

active ingredient: 5 to 75%, preferably 10 to 50%

water: 94 to 24%, preferably 88 to 30%

surface-active agent: 1 to 40%, preferably 2 to 30%

Wettable Powders:

active ingredient: 0.5 to 90%, preferably 1 to 80%

surface-active agent: 0.5 to 20%, preferably 1 to 15%

solid carrier: 5 to 95%, preferably 15 to 90%

Granules:

active ingredient: 0.1 to 30%, preferably 0.1 to 15%

solid carrier: 99.5 to 70%, preferably 97 to 85%

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether (7-8 mol | — | 2% | — |
| of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | — | 20% |

The combination is thoroughly mixed with the adjuvants and the mixture is thoroughly ground in a suitable mill, affording powders that can be used directly for seed treatment.

| Emulsifiable concentrate | |
|---|---|
| active ingredients | 10% |
| octylphenol polyethylene glycol ether (4-5 mol of ethylene | 3% |
| oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

| Suspension concentrate | |
|---|---|
| active ingredients | 40% |
| propylene glycol | 10% |
| nonylphenol polyethylene glycol ether (15 mol | 6% |
| of ethylene oxide) | |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion. PGP-53 TI

| Flowable concentrate for seed treatment | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form of a | 0.5% |
| 20% solution in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

In a further aspect, the present invention makes available a pesticidal composition comprising a compound of the first aspect, one or more formulation additives and a carrier.

The activity of the compositions according to the invention can be broadened considerably, and adapted to prevailing circumstances, by adding other insecticidally, acaricidally and/or fungicidally active ingredients. The mixtures of the compounds of formula (I) with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use.

Suitable additions to active ingredients here are, for example, representatives of the following classes of active ingredients: organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, neonicotinoids and Bacillus thuringiensis preparations.

The following mixtures of the compounds of formula (I) with active ingredients are preferred (the abbreviation “TX” means “one compound selected from the group consisting of a compound 1.001 to 1.105 listed in Table 1 (below) or a compound A1 to A102 listed in Table A (below):

an adjuvant selected from the group of substances consisting of petroleum oils (628)+TX,

an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chlorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate+TX, bromocyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chinomethionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin I (696)+TX, cinerin II (696)+TX, cinerins (696)+TX, clofentezine (158)+TX, closantel [CCN]+TX, coumaphos (174)+TX, crotamiton [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermethrin (201)+TX, DCPM (1032)+TX, DDT (219)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulfon (1039)+TX, diafenthiuron (226)+TX, dialifos (1042)+TX, diazinon (227)+TX, dichlofluanid (230)+TX, dichlorvos (236)+TX, dicliphos+TX, dicofol (242)+TX, dicrotophos (243)+TX, dienochlor (1071)+TX, dimefox (1081)+TX, dimethoate (262)+TX, dinactin (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram [CCN]+TX, disulfoton (278)+TX, DNOC (282)+TX, dofenapyn (1113)+TX, doramectin [CCN]+TX, endosulfan (294)+TX, endothion (1121)+TX, EPN (297)+TX, eprinomectin [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad+TX, fenpyroximate (345)+TX, fenson (1157)+TX, fentrifanil (1161)+TX, fenvalerate (349)+TX, fipronil (354)+TX, fluacrypyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (473)+TX, isopropyl 0-(methoxyaminothiophosphoryl)salicyiate (IUPAC name) (473)+TX, ivermectin [CCN]+TX, jasmolin I (696)+TX, jasmolin II (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime [CCN]+TX, mipafox (1293)+TX, monocrotophos (561)+TX, morphothion (1300)+TX, moxidectin [CCN]+TX, naled (567)+TX, NC-184 (compound code)+TX, NC-512 (compound code)+TX, nifluridide (1309)+TX, nikkomycins [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp′-DDT (219)+TX, parathion (615)+TX, permethrin (626)+TX, petroleum oils (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin I (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos+TX, selamectin [CCN]+TX, SI-0009 (compound code)+TX, sophamide (1402)+TX, spirodiclofen (738)+TX, spiromesifen (739)+TX, SSI-121 (development code) (1404)+TX, sulfiram [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam+TX, tetrachlorvinphos (777)+TX, tetradifon (786)+TX, tetranactin (653)+TX, tetrasul (1425)+TX, thiafenox+TX, thiocarboxime (1431)+TX, thiofanox (800)+TX, thiometon (801)+TX, thioquinox (1436)+TX, thuringiensin [CCN]+TX, triamiphos (1441)+TX, triarathene (1443)+TX, triazophos (820)+TX, triazuron+TX, trichlorfon (824)+TX, trifenofos (1455)+TX, trinactin (653)+TX, vamidothion (847)+TX, vaniliprole [CCN] and YI-5302 (compound code)+TX,

an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX,

an anthelmintic selected from the group of substances consisting of abamectin (1)+TX, crufomate (1011)+TX, doramectin [CCN]+TX, emamectin (291)+TX, emamectin benzoate (291)+TX, eprinomectin [CCN]+TX, ivermectin [CCN]+TX, milbemycin oxime [CCN]+TX, moxidectin [CCN]+TX, piperazine [CCN]+TX, selamectin [CCN]+TX, spinosad (737) and thiophanate (1435)+TX, an avicide selected from the group of substances consisting of chloralose (127)+TX, endrin (1122)+TX, fenthion (346)+TX, pyridin-4-amine (IUPAC name) (23) and strychnine (745)+TX,

a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal [CCN]+TX,

a biological agent selected from the group of substances consisting of Adoxophyes orana GV (12)+TX, Agrobacterium radiobacter (13)+TX, Amblyseius spp. (19)+TX, Anagrapha falcifera NPV (28)+TX, Anagrus atomus (29)+TX, Aphelinus abdominalis (33)+TX, Aphidius colemani (34)+TX, Aphidoletes aphidimyza (35)+TX, Autographa californica NPV (38)+TX, Bacillus firmus (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (53)+TX, Beauveria brongniartii (54)+TX, Chrysoperla carnea (151)+TX, Cryptolaemus montrouzieri (178)+TX, Cydia pomonella GV (191)+TX, Dacnusa sibirica (212)+TX, Diglyphus isaea (254)+TX, Encarsia formosa (scientific name) (293)+TX, Eretmocerus eremicus (300)+TX, Helicoverpa zea NPV (431)+TX, Heterorhabditis bacteriophora and H. megidis (433)+TX, Hippodamia convergens (442)+TX, Leptomastix dactylopii (488)+TX, Macrolophus caliginosus (491)+TX, Mamestra brassicae NPV (494)+TX, Metaphycus helvolus (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (575)+TX, Onus spp. (596)+TX, Paecilomyces fumosoroseus (613)+TX, Phytoseiulus persimilis (644)+TX, Spodoptera exigua multicapsid nuclear polyhedrosis virus (scientific name) (741)+TX, Steinernema bibionis (742)+TX, Steinernema carpocapsae (742)+TX, Steinernema feltiae (742)+TX, Steinernema glaseri (742)+TX, Steinernema nobrave (742)+TX, Steinernema riobravis (742)+TX, Steinernema scapterisci (742)+TX, Steinernema spp. (742)+TX, Trichogramma spp. (826)+TX, Typhlodromus occidentalis (844) and Verticillium lecanii (848)+TX,

a soil sterilant selected from the group of substances consisting of iodomethane (IUPAC name) (542) and methyl bromide (537)+TX,

a chemosterilant selected from the group of substances consisting of apholate [CCN]+TX, bisazir [CCN]+TX, busulfan [CCN]+TX, diflubenzuron (250)+TX, dimatif [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron [CCN]+TX, tepa [CCN]+TX, thiohempa [CCN]+TX, thiotepa [CCN]+TX, tretamine [CCN] and uredepa [CCN]+TX,

an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin [CCN]+TX, brevicomin [CCN]+TX, codlelure [CCN]+TX, codlemone (167)+TX, cuelure (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure [CCN]+TX, ethyl 4-methyloctanoate (IUPAC name) (317)+TX, eugenol [CCN]+TX, frontalin [CCN]+TX, gossyplure (420)+TX, grandlure (421)+TX, grandlure I (421)+TX, grandlure II (421)+TX, grandlure III (421)+TX, grandlure IV (421)+TX, hexalure [CCN]+TX, ipsdienol [CCN]+TX, ipsenol [CCN]+TX, japonilure (481)+TX, lineatin [CCN]+TX, litlure [CCN]+TX, looplure [CCN]+TX, medlure [CCN]+TX, megatomoic acid [CCN]+TX, methyl eugenol (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure [CCN]+TX, oryctalure (317)+TX, ostramone [CCN]+TX, siglure [CCN]+TX, sordidin (736)+TX, sulcatol [CCN]+TX, tetradec-11-en-1-yl acetate (IUPAC name) (785)+TX, trimedlure (839)+TX, trimedlure A (839)+TX, trimedlure B₁ (839)+TX, trimedlure B₂ (839)+TX, trimedlure C (839) and trunc-call [CCN]+TX,

an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX,

an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 1,2-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichlorophenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethylcarbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin [CCN]+TX, allyxycarb (866)+TX, alpha-cypermethrin (202)+TX, alpha-ecdysone [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (41)+TX, azamethiphos (42)+TX, azinphos-ethyl (44)+TX, azinphos-methyl (45)+TX, azothoate (889)+TX, Bacillus thuringiensis delta endotoxins (52)+TX, barium hexafluorosilicate [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrin (194)+TX, beta-cypermethrin (203)+TX, bifenthrin (76)+TX, bioallethrin (78)+TX, bioallethrin S-cyclopentenyl isomer (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate+TX, bromfenvinfos (914)+TX, bromocyclen (918)+TX, bromo-DDT [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, chloroform [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin I (696)+TX, cinerin II (696)+TX, cinerins (696)+TX, cis-resmethrin+TX, cismethrin (80)+TX, clocythrin+TX, cloethocarb (999)+TX, closantel [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton [CCN]+TX, crotoxyphos (1010)+TX, crufomate (1011)+TX, cryolite (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate [CCN]+TX, d-limonene [CCN]+TX, d-tetramethrin (788)+TX, DAEP (1031)+TX, dazomet (216)+TX, DDT (219)+TX, decarbofuran (1034)+TX, deltamethrin (223)+TX, demephion (1037)+TX, demephion-0 (1037)+TX, demephion-S (1037)+TX, demeton (1038)+TX, demeton-methyl (224)+TX, demeton-0 (1038)+TX, demeton-O-methyl (224)+TX, demeton-S (1038)+TX, demeton-S-methyl (224)+TX, demeton-S-methylsulphon (1039)+TX, diafenthiuron (226)+TX, dialifos (1042)+TX, diamidafos (1044)+TX, diazinon (227)+TX, dicapthon (1050)+TX, dichlofenthion (1051)+TX, dichlorvos (236)+TX, dicliphos+TX, dicresyl [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin [CCN]+TX, DSP (1115)+TX, ecdysterone [CCN]+TX, El 1642 (development code) (1118)+TX, emamectin (291)+TX, emamectin benzoate (291)+TX, EMPC (1120)+TX, empenthrin (292)+TX, endosulfan (294)+TX, endothion (1121)+TX, endrin (1122)+TX, EPBP (1123)+TX, EPN (297)+TX, epofenonane (1124)+TX, eprinomectin [CCN]+TX, esfenvalerate (302)+TX, etaphos [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (473)+TX, isodrin (1235)+TX, isofenphos (1236)+TX, isolane (1237)+TX, isoprocarb (472)+TX, isopropyl 0-(methoxyaminothiophosphoryl)salicylate (IUPAC name) (473)+TX, isoprothiolane (474)+TX, isothioate (1244)+TX, isoxathion (480)+TX, ivermectin [CCN]+TX, jasmolin I (696)+TX, jasmolin II (696)+TX, jodfenphos (1248)+TX, juvenile hormone I [CCN]+TX, juvenile hormone II [CCN]+TX, juvenile hormone III [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (533)+TX, methoxychlor (534)+TX, methoxyfenozide (535)+TX, methyl bromide (537)+TX, methyl isothiocyanate (543)+TX, methylchloroform [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime [CCN]+TX, mipafox (1293)+TX, mirex (1294)+TX, monocrotophos (561)+TX, morphothion (1300)+TX, moxidectin [CCN]+TX, naftalofos [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, O-5-dichloro-4-iodophenyl O-ethyl ethylphosphonothioate (IUPAC name) (1057)+TX, O,O-diethyl O-4-methyl-2-oxo-2H-chromen-7-yl phosphorothioate (IUPAC name) (1074)+TX, O,O-diethyl O-6-methyl-2-propylpyrimidin-4-yl phosphorothioate (IUPAC name) (1075)+TX, O,O,O′,O′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron [CCN]+TX, pentachlorophenol (623)+TX, pentachlorophenyl laurate (IUPAC name) (623)+TX, permethrin (626)+TX, petroleum oils (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, potassium arsenite [CCN]+TX, potassium thiocyanate [CCN]+TX, prallethrin (655)+TX, precocene I [CCN]+TX, precocene II [CCN]+TX, precocene III [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia [CCN]+TX, quinalphos (711)+TX, quinalphos-methyl (1376)+TX, quinothion (1380)+TX, quintiofos (1381)+TX, R-1492 (development code) (1382)+TX, rafoxanide [CCN]+TX, resmethrin (719)+TX, rotenone (722)+TX, RU 15525 (development code) (723)+TX, RU 25475 (development code) (1386)+TX, ryania (1387)+TX, ryanodine (traditional name) (1387)+TX, sabadilla (725)+TX, schradan (1389)+TX, sebufos+TX, selamectin [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spiropidion (CCN)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron+TX, trichlorfon (824)+TX, trichlormetaphos-3 [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (725)+TX, veratrine (725)+TX, XMC (853)+TX, xylylcarb (854)+TX, YI-5302 (compound code)+TX, zeta-cypermethrin (205)+TX, zetamethrin+TX, zinc phosphide (640)+TX, zolaprofos (1469) and ZXI 8901 (development code) (858)+TX, cyantraniliprole [736994-63-19+TX, chlorantraniliprole [500008-45-7]+TX, cyenopyrafen [560121-52-0]+TX, cyflumetofen [400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole [704886-18-0]+TX, meperfluthrin [915288-13-0]+TX, tetramethylfluthrin [84937-88-2]+TX, triflumezopyrim (disclosed in WO 2012/092115)+TX,

a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX,

a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (210)+TX, dazomet (216)+TX, DBCP (1045)+TX, DCIP (218)+TX, diamidafos (1044)+TX, dichlofenthion (1051)+TX, dicliphos+TX, dimethoate (262)+TX, doramectin [CCN]+TX, emamectin (291)+TX, emamectin benzoate (291)+TX, eprinomectin [CCN]+TX, ethoprophos (312)+TX, ethylene dibromide (316)+TX, fenamiphos (326)+TX, fenpyrad+TX, fensulfothion (1158)+TX, fosthiazate (408)+TX, fosthietan (1196)+TX, furfural [CCN]+TX, GY-81 (development code) (423)+TX, heterophos [CCN]+TX, iodomethane (IUPAC name) (542)+TX, isamidofos (1230)+TX, isazofos (1231)+TX, ivermectin [CCN]+TX, kinetin (210)+TX, mecarphon (1258)+TX, metam (519)+TX, metam-potassium (519)+TX, metam-sodium (519)+TX, methyl bromide (537)+TX, methyl isothiocyanate (543)+TX, milbemycin oxime [CCN]+TX, moxidectin [CCN]+TX, Myrothecium verrucaria composition (565)+TX, NC-184 (compound code)+TX, oxamyl (602)+TX, phorate (636)+TX, phosphamidon (639)+TX, phosphocarb [CCN]+TX, sebufos+TX, selamectin [CCN]+TX, spinosad (737)+TX, terbam+TX, terbufos (773)+TX, tetrachlorothiophene (IUPAC/Chemical Abstracts name) (1422)+TX, thiafenox+TX, thionazin (1434)+TX, triazophos (820)+TX, triazuron+TX, xylenols [CCN]+TX, YI-5302 (compound code) and zeatin (210)+TX, fluensulfone [318290-98-1]+TX,

a nitrification inhibitor selected from the group of substances consisting of potassium ethylxanthate [CCN] and nitrapyrin (580)+TX,

a plant activator selected from the group of substances consisting of acibenzolar (6)+TX, acibenzolar-S-methyl (6)+TX, probenazole (658) and Reynoutria sachalinensis extract (720)+TX,

a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX,

a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,

an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX,

a virucide selected from the group of substances consisting of imanin [CCN] and ribavirin [CCN]+TX,

a wound protectant selected from the group of substances consisting of mercuric oxide (512)+TX, octhilinone (590) and thiophanate-methyl (802)+TX,

and biologically active compounds selected from the group consisting of azaconazole (60207-31-0]+TX, bitertanol [70585-36-3]+TX, bromuconazole [116255-48-2]+TX, cyproconazole [94361-06-5]+TX, difenoconazole [119446-68-3]+TX, diniconazole [83657-24-3]+TX, epoxiconazole [106325-08-0]+TX, fenbuconazole [114369-43-6]+TX, fluquinconazole [136426-54-5]+TX, flusilazole [85509-19-9]+TX, flutriafol [76674-21-0]+TX, hexaconazole [79983-71-4]+TX, imazalil [35554-44-0]+TX, imibenconazole [86598-92-7]+TX, ipconazole [125225-28-7]+TX, metconazole [125116-23-6]+TX, myclobutanil [88671-89-0]+TX, pefurazoate [101903-30-4]+TX, penconazole [66246-88-6]+TX, prothioconazole [178928-70-6]+TX, pyrifenox [88283-41-4]+TX, prochloraz [67747-09-5]+TX, propiconazole [60207-90-1]+TX, simeconazole [149508-90-7]+TX, tebuconazole [107534-96-3]+TX, tetraconazole [112281-77-3]+TX, triadimefon [43121-43-3]+TX, triadimenol [55219-65-3]+TX, triflumizole [99387-89-0]+TX, triticonazole [131983-72-7]+TX, ancymidol [12771-68-5]+TX, fenarimol [60168-88-9]+TX, nuarimol [63284-71-9]+TX, bupirimate [41483-43-6]+TX, dimethirimol [5221-53-4]+TX, ethirimol [23947-60-6]+TX, dodemorph [1593-77-7]+TX, fenpropidine [67306-00-7]+TX, fenpropimorph [67564-91-4]+TX, spiroxamine [118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl [71626-11-4]+TX, furalaxyl [57646-30-7]+TX, metalaxyl [57837-19-1]+TX, R-metalaxyl [70630-17-0]+TX, ofurace [58810-48-3]+TX, oxadixyl [77732-09-3]+TX, benomyl [17804-35-2]+TX, carbendazim [10605-21-7]+TX, debacarb [62732-91-6]+TX, fuberidazole [3878-19-1]+TX, thiabendazole [148-79-8]+TX, chlozolinate [84332-86-5]+TX, dichlozoline [24201-58-9]+TX, iprodione [36734-19-7]+TX, myclozoline [54864-61-8]+TX, procymidone [32809-16-8]+TX, vinclozoline [50471-44-8]+TX, boscalid[188425-85-6]+TX, carboxin [5234-68-4]+TX, fenfuram [24691-80-3]+TX, flutolanil [66332-96-5]+TX, mepronil [55814-41-0]+TX, oxycarboxin [5259-88-1]+TX, penthiopyrad [183675-82-3]+TX, thifluzamide [130000-40-7]+TX, guazatine [108173-90-6]+TX, dodine [2439-10-3] [112-65-2](free base)+TX, iminoctadine [13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl [143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol [2425-06-1]+TX, captan [133-06-2]+TX, dichlofluanid [1085-98-9]+TX, fluoroimide [41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper [53988-93-5]+TX, oxine-copper [10380-28-6]+TX, dinocap [131-72-6]+TX, nitrothal-isopropyl [10552-74-6]+TX, edifenphos [17109-49-8]+TX, iprobenphos [26087-47-8]+TX, isoprothiolane [50512-35-1]+TX, phosdiphen [36519-00-3]+TX, pyrazophos [13457-18-6]+TX, tolclofos-methyl [57018-04-9]+TX, acibenzolar-S-methyl [135158-54-2]+TX, anilazine [101-05-3]+TX, benthiavalicarb [413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone [117-80-6]+TX, diclocymet [139920-32-4]+TX, diclomezine [62865-36-5]+TX, dicloran [99-30-9]+TX, diethofencarb [87130-20-9]+TX, dimethomorph [110488-70-5]+TX, SYP-LI90 (Flumorph) [211867-47-9]+TX, dithianon [3347-22-6]+TX, ethaboxam [162650-77-3]+TX, etridiazole [2593-15-9]+TX, famoxadone [131807-57-3]+TX, fenamidone [161326-34-7]+TX, fenoxanil [115852-48-7]+TX, fentin [668-34-8]+TX, ferimzone [89269-64-7]+TX, fluazinam [79622-59-6]+TX, fluopicolide [239110-15-7]+TX, flusulfamide [106917-52-6]+TX, fenhexamid [126833-17-8]+TX, fosetyl-aluminium [39148-24-8]+TX, hymexazol [10004-44-1]+TX, iprovalicarb [140923-17-7]+TX, IKF-916 (Cyazofamid) [120116-88-3]+TX, kasugamycin [6980-18-3]+TX, methasulfocarb [66952-49-6]+TX, metrafenone [220899-03-6]+TX, pencycuron [66063-05-6]+TX, phthalide [27355-22-2]+TX, polyoxins [11113-80-7]+TX, probenazole [27605-76-1]+TX, propamocarb [25606-41-1]+TX, proquinazid [189278-12-4]+TX, pyroquilon [57369-32-1]+TX, quinoxyfen [124495-18-7]+TX, quintozene [82-68-8]+TX, sulfur [7704-34-9]+TX, tiadinil [223580-51-6]+TX, triazoxide [72459-58-6]+TX, tricyclazole [41814-78-2]+TX, triforine [26644-46-2]+TX, validamycin [37248-47-8]+TX, zoxamide (RH7281) [156052-68-5]+TX, mandipropamid [374726-62-2]+TX, isopyrazam [881685-58-1]+TX, sedaxane [874967-67-6]+TX, 3-difluoromethyl-1-methyl-1H-pyrazole-4-carboxylic acid (9-dichloromethylene-1,2,3,4-tetrahydro-1,4-methano-naphthalen-5-yl)-amide (dislosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R, 12aS, 12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11Hnaphtho[2,1-b]pyrano[3,4-e]pyran-4-yl]methyl-cyclopropanecarboxylate [915972-17-7]+TX and 1,3,5-trimethyl-N-(2-methyl-1-oxopropyl)-N-[3-(2-methylpropyl)-4-[2,2,2-trifluoro-1-m ethoxy-1-(trifluoromethyl)ethyl]phenyl]-1H-pyrazole-4-carboxamide [926914-55-8]+TX, lancotrione [1486617-21-3]+TX, florpyrauxifen [943832-81-3]]+TX, ipfentrifluconazole[1417782-08-1]+TX, mefentrifluconazole [1417782-03-6]+TX, quinofumelin [861647-84-9]+TX, chloroprallethrin [399572-87-3]+TX, cyhalodiamide [1262605-53-7]]+TX, fluazaindolizine [1254304-22-7]+TX, fluxametamide [928783-29-3]+TX, epsilon-metofluthrin [240494-71-7]+TX, epsilon-momfluorothrin [1065124-65-3]+TX, pydiflumetofen [1228284-64-7]+TX, kappa-bifenthrin [439680-76-9]+TX, broflanilide [1207727-04-5]+TX, dicloromezotiaz [1263629-39-5]+TX, dipymetitrone [16114-35-5]+TX, pyraziflumid [942515-63-1]+TX, kappa-tefluthrin [391634-71-2]+TX, fenpicoxamid [517875-34-2]+TX, fluindapyr [1383809-87-7]+TX, alpha-bromadiolone [28772-56-7]+TX, flupyrimin [1689566-03-7]+TX, benzpyrimoxan [1449021-97-9]+TX, acynonapyr [1332838-17-1]+TX, inpyrfluxam [1352994-67-2]+TX, isoflucypram [1255734-28-1]+TX, rescalure [64309-03-1]+TX, aminopyrifen [1531626-08-0]+TX, tyclopyrazoflor [1477919-27-9]+TX, Dichloromezotiaz+TX, Momfluorothrin+TX, Fluopyram+TX, Tioxazafen+TX, Terpenoid blend+TX, Fluhexafon+TX, Cyclaniliprole+TX, and spiropidion [1229023-00-0]+TX; and

microbials including: Acinetobacter Iwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®, BioNem-WP®, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1 Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmominiatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HC®+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus, and

Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®)+TX, and

pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC-LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, Tetradecatrienyl acetate+TX, 13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate, and

Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline cucumeris®)+TX, Amblyseius fallacis (Fallacis®)+TX, Amblyseius swirskii (Bugline swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperia carnea (Chrysoline®)+TX, Chrysoperia carnea (Chrysopa®)+TX, Chrysoperia rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline E®)+TX, Eretmocerus eremicus (Bemimix®)+TX, Eretmocerus hayati+TX, Eretmocerus mundus (Bemipar®+TX, Eretline M®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline Hm®+TX, Larvanem-M®)+TX, Hippodamia convergens+TX, Hypoaspis aculeifer (Aculeifer-System®+TX, Entomite-A®)+TX, Hypoaspis miles (Hypoline M®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline C®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Onus insidiosus (Thripor-I®+TX, Oriline I®)+TX, Onus laevigatus (Thripor-L®+TX, Oriline I®)+TX, Onus majusculus (Oriline M®)+TX, Onus strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline P®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline Sf®+TX, Scia-rid®+TX, Entonem®)+TX, Steinernema kraussei (Nemasys L®+TX, BioNem L®+TX, Exhibitline Srb®)+TX, Steinernema nobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine B®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator, and

other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline D®)+TX, Erwinia amylovora (Harpin) (ProAct®+TX, Ni-HIBIT Gold CST®)+TX, Ferri-phosphate (Ferramol®)+TX, Funnel traps (Trapline Y®)+TX, Gallex®+TX, Grower's Secret®+TX, Homo-brassonolide+TX, Iron Phosphate (Lilly Miller Worry Free Ferramol Slug & Snail Bait®)+TX, MOP hail trap (Trapline F®)+TX, Microctonus hyperodae+TX, Mycoleptodiscus terrestris (Des-X®)+TX, BioGain®+TX, Aminomite®+TX, Zenox®+TX, Pheromone trap (Thripline Ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+B®)+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium, Thirteenth Edition, Editor: C. D. S. TomLin, The British Crop Protection Council], they are described therein under the entry number given in round brackets hereinabove for the particular compound, for example, the compound “abamectin” is described under entry number (1). Where “[CCN]” is added hereinabove to the particular compound, the compound in question is included in the “Compendium of Pesticide Common Names”, which is accessible on the internet [A. Wood, Compendium of Pesticide Common Names, Copyright © 1995-2004], for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound, in that case, the IUPAC name, the IUPAC/Chemical Abstracts name, a “chemical name”, a “traditional name”, a “compound name” or a “development code” is used. “CAS Reg. No” means the Chemical Abstracts Registry Number.

The ratio (by weight) of active ingredient mixture of the compounds of formula (I) selected from a compound 1.001 to 1.105 listed in Table 1 (below) or a compound A1 to A102 listed in Table A (below) with active ingredients described above is from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula (I) selected from a compound 1.001 to 1.105 listed in Table 1 (below) or a compound A1 to A102 listed in Table A (below) and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula (I) selected from a compound 1.001 to 1.105 listed in Table 1 (below) or a compound A1 to A102 listed in Table A (below) and the active ingredients as described above is not essential for working the present invention.

In a further aspect, the present invention provides a combination of active ingredients comprising a compound defined in the first aspect, and one or more further active ingredients (whether chemical or biological).

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds (I) for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula (I). The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

A further aspect is a plant propagation material comprising by way of treatment or coating one or more compounds of formula (I) according to the invention, optionally also comprising a colour pigment.

In each aspect and embodiment of the invention, “consisting essentially” and inflections thereof are a preferred embodiment of “comprising” and its inflections, and “consisting of” and inflections thereof are a preferred embodiment of “consisting essentially of” and its inflections.

The disclosure in the present application makes available each and every combination of embodiments disclosed herein.

Table 1: This table discloses the 105 compounds of the formula (I-1):

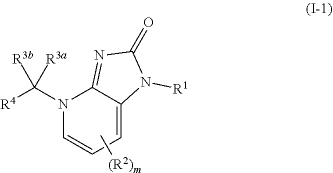

wherein m is 0, R^{3a} and R^{3b} are hydrogen, and R¹ and R⁴ are as defined in the below Table.

| Compound | | |
|---|---|---|
| no. | R⁴ | R¹ |
| 1.001 | 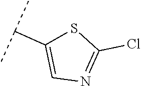 | phenyl |
| 1.002 | 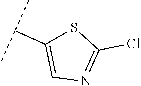 | 3- cyanophenyl |
| 1.003 | 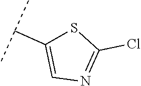 | 2-fluoro- phenyl |
| 1.004 | 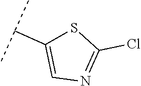 | pyridin-2-yl |
| 1.005 | 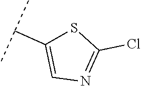 | pyridin-3-yl |
| 1.006 | 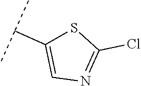 | 5-fluoro- pyridin-3-yl |
| 1.007 | 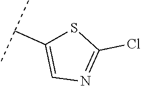 | thien-2-yl |
| 1.008 | 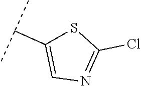 | 4-cyano- pyridin-2-yl |
| 1.009 | 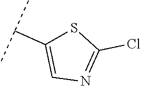 | 5-cyano- pyridin-2-yl |
| 1.010 | 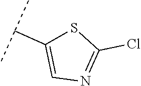 | 3-cyano- pyridin-2-yl |
| 1.011 | 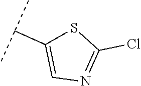 | pyridin-4-yl |
| 1.012 | 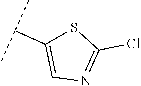 | pyrimidin-2-yl |
| 1.013 | 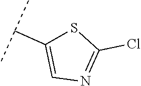 | 3-phenyl- phenyl |
| 1.014 | 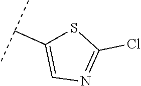 | quinolin-3-yl |
| 1.015 | 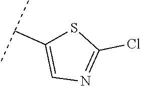 | 1-methyl- pyrazol-4-yl |
| 1.016 | 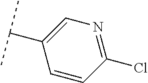 | phenyl |
| 1.017 | 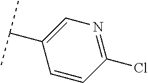 | 3-cyanophenyl |
| 1.018 | 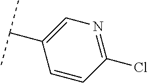 | 2-fluoro- phenyl |
| 1.019 | 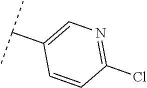 | pyridin-2-yl |
| 1.020 | 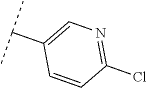 | pyridin-3-yl |
| 1.021 | 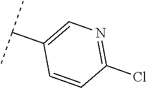 | 5-fluoro- pyridin-3-yl |
| 1.022 | 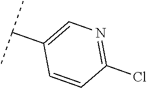 | thien-2-yl |
| 1.023 | 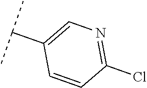 | 4-cyano- pyridin-2-yl |
| 1.024 | 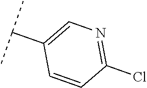 | 5-cyano- pyridin-2-yl |
| 1.025 | 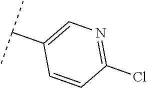 | 3-cyano- pyridin-2-yl |
| 1.026 | 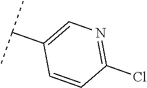 | pyridin-4-yl |
| 1.027 | 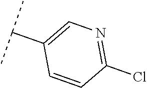 | pyrimidin-2-yl |
| 1.028 | 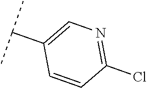 | 3-phenyl- phenyl |
| 1.029 | 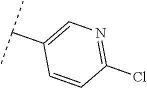 | quinolin-3-yl |
| 1.030 | 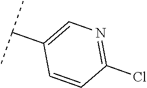 | 1-methyl- pyrazol-4-yl |
| 1.031 | 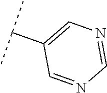 | phenyl |
| 1.032 | 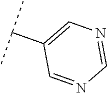 | 3-cyano- phenyl |
| 1.033 | 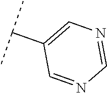 | 2-fluoro- phenyl |
| 1.034 | 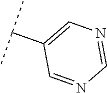 | pyridin-2-yl |
| 1.035 | 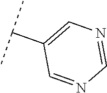 | pyridin-3-yl |
| 1.036 | 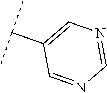 | 5-fluoro- pyridin-3-yl |
| 1.037 | 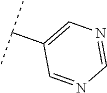 | thien-2-yl |
| 1.038 | 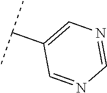 | 4-cyano- pyridin-2-yl |
| 1.039 | 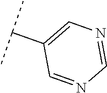 | 5-cyano- pyridin-2-yl |
| 1.040 | 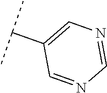 | 3-cyano- pyridin-2-yl |
| 1.041 | 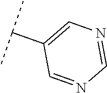 | pyridin-4-yl |
| 1.042 | 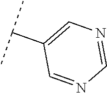 | pyrimidin-2-yl |
| 1.043 | 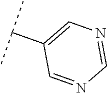 | 3-phenyl- phenyl |
| 1.044 | 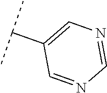 | quinolin-3-yl |
| 1.045 | 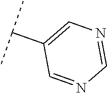 | 1-methyl- pyrazol-4-yl |
| 1.046 | 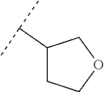 | phenyl |
| 1.047 | 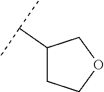 | 3-cyanophenyl |
| 1.048 | 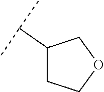 | 2-fluoro- phenyl |
| 1.049 | 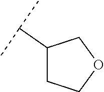 | pyridin-2-yl |
| 1.050 | 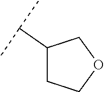 | pyridin-3-yl |
| 1.051 | 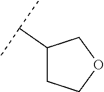 | 5-fluoro- pyridin-3-yl |
| 1.052 | 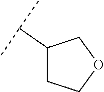 | thien-2-yl |
| 1.053 | 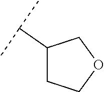 | 4-cyano- pyridin-2-yl |
| 1.054 | 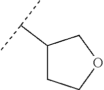 | 5-cyano- pyridin-2-yl |
| 1.055 | 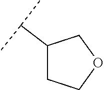 | 3-cyano- pyridin-2-yl |
| 1.056 | 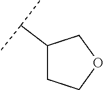 | pyridin-4-yl |
| 1.057 | 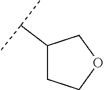 | pyrimidin-2-yl |
| 1.058 | 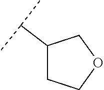 | 3-phenyl- phenyl |
| 1.059 | 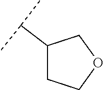 | quinolin-3-yl |
| 1.060 | 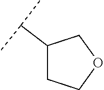 | 1-methyl- pyrazol-4-yl |
| 1.061 | 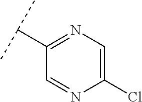 | phenyl |
| 1.062 | 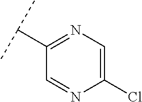 | 3-cyano- phenyl |
| 1.063 | 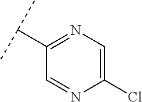 | 2-fluoro- phenyl |
| 1.064 | 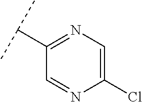 | pyridin-2-yl |
| 1.065 | 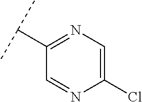 | pyridin-3-yl |
| 1.066 | 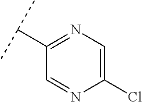 | 5-fluoro- pyridin-3-yl |
| 1.067 | 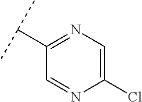 | thien-2-yl |
| 1.068 | 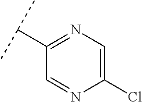 | 4-cyano- pyridin-2-yl |
| 1.069 | 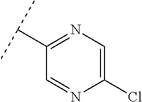 | 5-cyano- pyridin-2-yl |
| 1.070 | 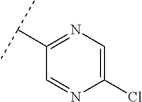 | 3-cyano- pyridin-2-yl |
| 1.071 | 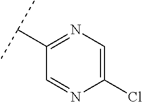 | pyridin-4-yl |
| 1.072 | 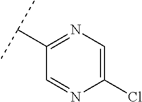 | pyrimidin-2-yl |
| 1.073 | 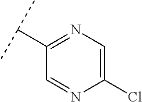 | 3-phenyl- phenyl |
| 1.074 | 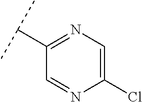 | quinolin-3-yl |
| 1.075 | 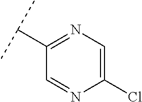 | 1-methyl- pyrazol-4-yl |
| 1.076 | 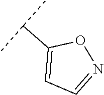 | phenyl |
| 1.077 | 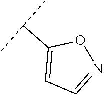 | 3-cyano- phenyl |
| 1.078 | 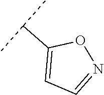 | 2-fluoro- phenyl |
| 1.079 | 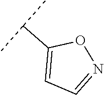 | pyridin-2-yl |
| 1.080 | 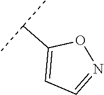 | pyridin-3-yl |
| 1.081 | 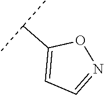 | 5-fluoro- pyridin-3-yl |
| 1.082 | 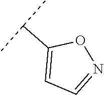 | thien-2-yl |
| 1.083 | 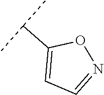 | 4-cyano- pyridin-2-yl |
| 1.084 | 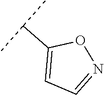 | 5-cyano- pyridin-2-yl |
| 1.085 | 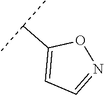 | 3-cyano- pyridin-2-yl |
| 1.086 | 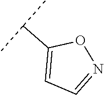 | pyridin-4-yl |
| 1.087 | 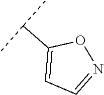 | pyrimidin-2-yl |
| 1.088 | 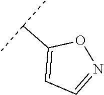 | 3-phenyl- phenyl |
| 1.089 | 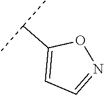 | quinolin-3-yl |
| 1.090 | 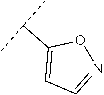 | 1-methyl- pyrazol-4-yl |
| 1.091 | 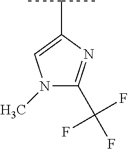 | phenyl |
| 1.092 | 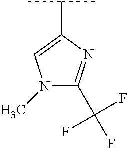 | 3-cyano- phenyl |
| 1.093 | 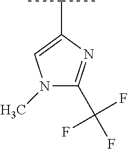 | 2-fluoro- phenyl |
| 1.094 | 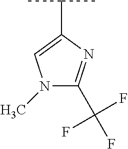 | pyridin-2-yl |
| 1.095 | 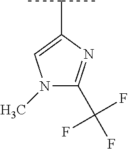 | pyridin-3-yl |
| 1.096 | 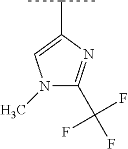 | 5-fluoro- pyridin-3-yl |
| 1.097 | 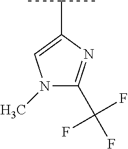 | thien-2-yl |
| 1.098 | 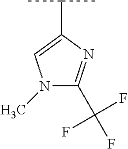 | 4-cyano- pyridin-2-yl |
| 1.099 | 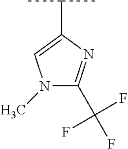 | 5-cyano- pyridin-2-yl |
| 1.100 | 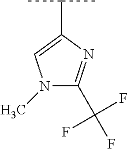 | 3-cyano- pyridin-2-yl |
| 1.101 | 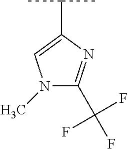 | pyridin-4-yl |
| 1.102 | 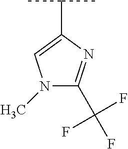 | pyrimidin-2-yl |
| 1.103 | 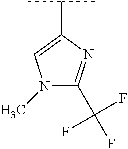 | 3-phenyl- phenyl |
| 1.104 | 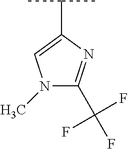 | quinolin-3-yl |
| 1.105 | 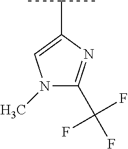 | 1-methyl- pyrazol-4-yl |

### EXAMPLES

The Examples which follow serve to illustrate the invention.

The compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm or 0.2 ppm, or lower application rates, such as 300, 200 or 100 mg of Al per m².

Compounds of Formula (I) may possess any number of benefits including, inter alia, advantageous levels of biological activity for protecting plants against insects or superior properties for use as agrochemical active ingredients (for example, greater biological activity, an advantageous spectrum of activity, an increased safety profile (including improved crop tolerance), improved physico-chemical properties, or increased biodegradability).

Throughout this description, temperatures are given in degrees Celsius (° C.) and “mp.” means melting point.

LC/MS means Liquid Chromatography Mass Spectrometry and the description of the apparatus and the method A is outlined below. The characteristic LC/MS values obtained for each compound were the retention time (“Rt”, recorded in minutes (min)) and the measured molecular ion (M+H)⁺ and/or (M−H)⁻.

¹H NMR measurements were recorded on Brucker 400 MHz or 300 MHz spectrometers, chemical shifts are given in ppm relevant to a TMS standard. Spectra are measured in deuterated solvents (eg, dimethyl sulfoxide (DMSO)) as indicated.

Method A—Standard

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLCfrom Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min) 0.85.

Method B.

Spectra were recorded on a ACQUITY Mass Spectrometer from Waters Corporations (SQD or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLCfrom Waters Corporations with solvent degasser, binary pump, heated column compartment and diode-array detector. Column: Waters UPLC HSS T3, 1.8 μm, 30×2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 2.5 min; Flow (ml/min) 0.75.

### Example P1: Preparation of 4-[(6-chloro-3-pyridyl)methyl]-1-phenyl-imidazo[4,5-b]pyridin-2-one (Compound A34)

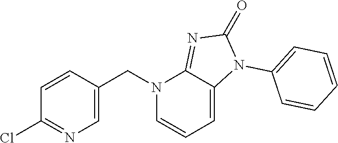 [SMILES: O=c1nc2n(Cc3ccc(Cl)nc3)cccc-2n1-c1ccccc1]

### Step A: Preparation of 1,3-dihydro-[1,2,5]thiadiazolo[3,4-b]pyridine 2,2-dioxide

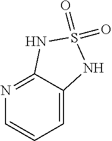 [SMILES: O=S1(=O)Nc2cccnc2N1]

To a solution of 2,3-diaminopyridine (10 g, 87.1 mmol) in pyridine (348 mL) was added sulfamide (12.7 g, 130.6 mmol) and the mixture was refluxed for 1.5 hour. After cooling, the pyridine was removed under reduced pressure and residual pyridine removed by azeotrope evaporation using toluene to give a crude black solid. Purification by chromatography on silica gel (dichloromethane/methanol gradient, 95:5→90:10) afforded 1,3-dihydro-[1,2,5]thiadiazolo[3,4-b]pyridine 2,2-dioxide (8.6 g, 58%) as a yellow solid.

¹H-NMR (400 MHz, DMSO-d₆): δ ppm 7.17 (dd, J=1.2, 6.6 Hz, 1H), 6.77 (dd, J=1.2, 7.2 Hz, 1H), 6.49-6.42 (m, 1H).

LCMS (method A): Rₜ=0.16 min, m/z=172 (M+H⁺).

### Step B: Preparation of tert-butyl 2,2-dioxo-3H-[1,2,5]thiadiazolo[3,4-b]pyridine-1-carboxylate

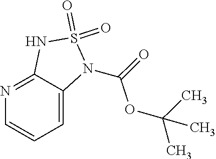 [SMILES: CC(C)(C)OC(=O)N1c2cccnc2NS1(=O)=O]

To a solution of 1,3-dihydro-[1,2,5]thiadiazolo[3,4-b]pyridine 2,2-dioxide (8.6 g, 50.2 mmol) in DMF (167 mL) was added 60% NaH in paraffin oil (2.2 g, 55.26 mmol). The suspension was stirred at room temperature for one hour, and a solution of (Boc)₂O (12.2 g, 55.26 mmol) in DMF (40 mL) was added dropwise. The reaction mixture was then stirred overnight and most of the solvent was removed at the rotavapor under reduced pressure. The residue was diluted with ethyl acetate (50 mL) and a saturated aqueous solution of ammonium chloride (50 mL) and the pH of the aqueous layer was adjusted to pH=5 to 6 with hydrochloric acid. The phases were separated and the aqueous phase was extracted with ethyl acetate (10 mL) and the combined organic phases were washed with water (2×10 mL), brine (10 mL), and dried with sodium sulfate, filtered and evaporated to give a crude black residue. Purification by chromatography on silica gel (cyclohexane/ethyl acetate gradient, 1:1->0:1) afforded tert-butyl 2,2-dioxo-3H-[1,2,5]thiadiazolo[3,4-b]pyridine-1-carboxylate (9.9 g, 73%) as a light yellow solid.

¹H-NMR (400 MHz, DMSO-d₆): δ ppm 13.75 (br s, 1H), 7.59 (dd, J=1.2, 7.4 Hz, 1H), 7.48 (dd, J=1.2, 6.6 Hz, 1H), 6.68 (dd, J=6.6, 7.4 Hz, 1H), 1.54 (s, 9H).

LCMS (method A): Rₜ=0.75 min, m/z=272 (M+H⁺).

### Step C: Preparation of tert-butyl 4-[(6-chloro-3-pyridyl)methyl]-2,2-dioxo-[1,2,5]thiadiazolo[3,4-b]pyridine-1-carboxylate

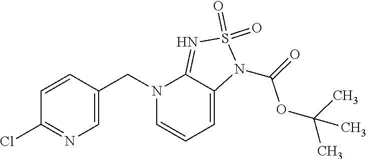 [SMILES: CC(C)(C)OC(=O)N1C2=CC=CN(Cc3ccc(Cl)nc3)C2=NS1(=O)=O]

To a solution of tert-butyl 2,2-dioxo-3H-[1,2,5]thiadiazolo[3,4-b]pyridine-1-carboxylate (9.1 g, 33.6 mmol) in DMF (134 mL) was added potassium carbonate (13.9 g, 100.7 mmol) and 2-chloro-5-(chloromethyl)pyridine (10.88 g, 67.16 mmol). The resulting mixture was stirred at room temperature overnight and quenched with water (100 mL). The aqueous phase was extracted with ethyl acetate (2×30 mL), the combined organic phases were washed with water (2×10 mL), brine (10 mL), and dried with sodium sulfate, filtered and evaporated. Purification by chromatography on silica gel (cyclohexane/ethyl acetate gradient, 1:1->0:1) afforded tert-butyl 4-[(6-chloro-3-pyridyl)methyl]-2,2-dioxo-[1,2,5]thiadiazolo[3,4-b]pyridine-1-carboxylate (11.48 g, 86%) as a light yellow solid.

¹H-NMR (400 MHz, DMSO-d₆): δ ppm 8.50 (d, J=2.6 Hz, 1H), 7.88-7.83 (m, 2H), 7.62-7.55 (m, 2H), 6.79 (s, 1H), 5.40 (s, 2H), 1.54 (s, 9H).

LCMS (method A): Rₜ=0.94 min, m/z=397 (M+H⁺).

### Step D: Preparation of 4-[(6-chloro-3-pyridyl)methyl]-1H-[1,2,5]thiadiazolo[3,4-b]pyridine 2,2-dioxide

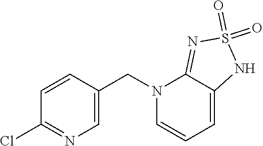 [SMILES: O=S1(=O)N=C2C(=CC=CN2Cc2ccc(Cl)nc2)N1]

A three-necked 200 mL flask fitted with a condenser was charged with tert-butyl 4-[(6-chloro-3-pyridyl)methyl]-2,2-dioxo-[1,2,5]thiadiazolo[3,4-b]pyridine-1-carboxylate (13.7 g, 33.1 mmol) in MeOH (300 mL). Acetyl chloride (7.17 mL, 99.4 mmol) was added dropwise and carefully (gas evolution during the addition) and the resulting yellow suspension was stirred at 65° C. for four hours. The reaction mixture was evaporated to give 4-[(6-chloro-3-pyridyl)methyl]-1H-[1,2,5]thiadiazolo[3,4-b]pyridine 2,2-dioxide (10.7 g, 100%) as a beige solid, which was used in the next step without any further purification.

¹H-NMR (400 MHz, DMSO-d₆): δ ppm 11.29 (br s, 1H), 8.50 (d, J=2.3 Hz, 1H), 7.86 (dd, J=2.3, 8.5 Hz, 1H), 7.57 (dd, J=1.1, 7.1 Hz, 1H), 7.56 (d, J=8.5 Hz, 1H), 6.77 (dd, J=1.1, 7.1 Hz, 1H), 6.55 (t, J=7.1 Hz, 1H), 5.35 (s, 2H).

LCMS (method A): Rₜ=0.57 min, m/z=297 (M+1).

### Step E: Preparation of 1-[(6-chloro-3-pyridyl)methyl]-2-imino-pyridin-3-amine

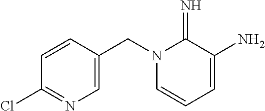 [SMILES: N=c1c(N)cccn1Cc1ccc(Cl)nc1]

A 25 mL microwave vial was charged with 4-[(6-chloro-3-pyridyl)methyl]-1H-[1,2,5]thiadiazolo[3,4-b]pyridine 2,2-dioxide (1 g, 2.36 mmol) and aqueous 37% HCl (6 mL). The vial was sealed and the purple mixture was stirred at 110° C. for 30 minutes. The reaction was cooled at 40° C. and the overpressure in the vial was released carefully. The vial was sealed again and the orange mixture was further stirred at 110° C. for two hours. The reaction mixture was cooled at room temperature, poured on ice, and basified to pH 10 with aqueous 10 M NaOH (8 mL). The aqueous phase was extracted with dichloromethane (4×30 mL), the combined organic phases were washed with water (5 mL), brine (5 mL), and dried with sodium sulfate, filtered and evaporated to give 1-[(6-chloro-3-pyridyl)methyl]-2-imino-pyridin-3-amine (270 mg, 49%) as an orange gum.

¹H-NMR (400 MHz, DMSO-d₆): δ ppm 8.37 (d, J=2.3 Hz, 1H), 7.74 (dd, J=2.3, 8.4 Hz, 1H), 7.47 (d, J=8.4 Hz, 1H), 6.86 (dd, J=1.5, 7.0 Hz, 1H), 6.25 (br s, 1H), 6.05 (dd, J=1.5, 7.0 Hz, 1H), 5.67 (t, J=7.0 Hz, 1H), 5.15-5.05 (m 4H).

LCMS (method A): Rₜ=0.25 min, m/z=235 (M+H⁺).

### Step F: Preparation of 4-[(6-chloro-3-pyridyl)methyl]-1H-imidazo[4,5-b]pyridin-2-one

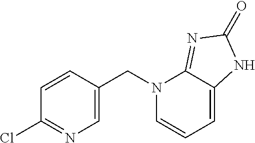 [SMILES: O=c1nc2n(Cc3ccc(Cl)nc3)cccc-2[nH]1]

To a solution of 1-[(6-chloro-3-pyridyl)methyl]-2-imino-pyridin-3-amine (715 mg, 2.59 mmol) in dichloromethane (30 mL) cooled at 0° C. was added phosgene (15% in toluene, 2.59 mL, 3.63 mmol) and the reaction mixture was stirred at room temperature overnight. The reaction was then cooled at 5° C., quenched with a 7 M solution of ammonia in MeOH (2 mL) and evaporated. Purification by chromatography on silica gel (dichloromethane/methanol, 90:10→80:20) afforded 4-[(6-chloro-3-pyridyl)methyl]-1H-imidazo[4,5-b]pyridin-2-one (600 mg, 90%).

¹H-NMR (400 MHz, DMSO-d₆): δ ppm 10.66 (bs, 1H), 8.54 (d, J=2.3 Hz, 1H), 7.89 (dd, J=2.3, 8.4 Hz, 1H), 7.73 (dd, J=1.0, 7.2 Hz, 1H), 7.53 (d, J=8.4 Hz, 1H), 6.97 (dd, J=1.0, 7.2 Hz, 1H), 6.69 (t, J=7.2 Hz, 1H), 5.49 (s, 2H).

LCMS (method A): Rₜ=0.30 min, m/z=261 (M+H⁺).

### Step G: Preparation of 4-[(6-chloro-3-pyridyl)methyl]-1-phenyl-imidazo[4,5-b]pyridin-2-one (Compound A34)

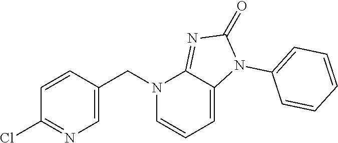 [SMILES: O=c1nc2n(Cc3ccc(Cl)nc3)cccc-2n1-c1ccccc1]

A 10 mL flask was charged with 4-[(6-chloro-3-pyridyl)methyl]-1H-imidazo[4,5-b]pyridin-2-one (200 mg, 0.70 mmol), acetonitrile (4 mL), cuprous iodide (44.7 mg, 0.23 mmol), potassium carbonate (268 mg, 1.92 mmol), N,N′-dimethylethylenediamine (41 mg, 0.46 mmol) and iodobenzene (237 mg, 1.15 mmol). The flask was fitted with a condenser and flushed with argon, and the resulting dark grey-blue suspension was stirred at 95° C. for eight hours. The dark grey-violet reaction mixture was passed through a pad of Celite which was carefully rinsed with MeOH (3×2 mL) and the remaining solution evaporated. Purification by chromatography on silica gel (dichloromethane/methanol gradient, 100:0→90:10) afforded 4-[(6-chloro-3-pyridyl)methyl]-1-phenyl-imidazo[4,5-b]pyridin-2-one as a light orange solid (195 mg, 75%).

¹H-NMR (400 MHz, CD₃OD): δ ppm 8.56 (d, J=2.6, 1H), 7.96 (dd, J=2.6, 8.4 Hz, 1H), 7.85 (dd, J=1.1, 7.0 Hz, 1H), 7.58-7.41 (m, 6H), 7.27 (dd, J=0.9, 7.5 Hz, 1H), 6.97-6.91 (m, 1H), 5.66 (s, 2H).

LCMS (method A): Rₜ=0.75 min, m/z=337 (M+H⁺).

### Example P2: Preparation of 1-phenyl-4-(pyrimidin-5-ylmethyl)imidazo[4,5-b]pyridin-2-one (Compound A35)

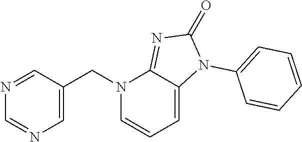 [SMILES: O=c1nc2n(Cc3cncnc3)cccc-2n1-c1ccccc1]

### Step A: preparation of 1,3-dihydroimidazo[4,5-b]pyridin-2-one

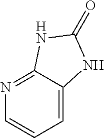

2-Aminopyridine-3-carboxylic acid (CAS 5345-47-1) (10.00 g, 72.40 mmol) was dispersed into 1,4-dioxane (300 mL) to obtain a light-brown suspension. Triethylamine (11.1 mL, 79.64 mmol) was then added slowly followed by diphenylphosphoryl azide (16.09 mL, 72.40 mmol) which was added dropwise over 15 minutes. The resulting light-brown suspension was slowly heated to reflux and stirred at reflux for 21 hours 30 minutes and allowed to cool to room temperature. The reaction medium was concentrated under reduced pressure at 40° C. to afford a brown oil. A minimum amount of MeOH was added and the precipitate formed was filtered through a sintered disc filter funnel. The solid press cake was washed with Et₂O and dried to afford 1,3-dihydroimidazo[4,5-b]pyridin-2-one. The filtrate was evaporated and purified by chromatography. Solids from the filtration and chromatography were mixed to afford 1,3-dihydroimidazo[4,5-b]pyridin-2-one.

¹H NMR (400 MHz, d⁶-DMSO): δ ppm 11.28 (br s, 1H), 10.81 (br s, 1H), 7.85 (dd, J=5.32, 1.28 Hz, 1H), 7.21 (dd, J=7.70, 1.10 Hz, 1H), 6.93 (dd, J=7.52, 5.32 Hz, 1H).

LC-MS (Method A): Rt=0.19 min, MS ES+=136 (M+H).

### Step B: Preparation of tert-butyl 2-oxo-3H-imidazo[4,5-b]pyridine-1-carboxylate

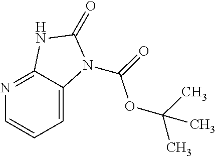 [SMILES: CC(C)(C)OC(=O)n1c(=O)[nH]c2ncccc21]

Under argon, 1,3-dihydroimidazo[4,5-b]pyridin-2-one (1.500 g, 11.10 mmol) was dissolved in DMF (20 mL). Then sodium hydride (0.4662 g, 11.66 mmol) was added portion-wise over 15 minutes. The resulting suspension was stirred at room temperature for 1 hour. Then di-tert-butyl dicarbonate (2.47 g, 11.10 mmol), dissolved into DMF (16.9 mL), was added dropwise over 10 minutes and the orange solution obtained was stirred at room temperature for 3 hours. Some MeOH was carefully added to quench the reaction mixture and then water was added. The pH of the solution was 8 to 9. The aqueous layer was extracted once with ethyl acetate and this extract was discarded (removal of impurities) then the pH was adjusted to 7 with 4 M aq. HCl solution and extraction was carried out with ethyl acetate. The combined organic layers were washed with water (×2), dried over anhydrous sodium sulfate, filtered and concentrated under reduced pressure at 40° C. to afford tert-butyl 2-oxo-3H-imidazo[4,5-b]pyridine-1-carboxylate.

¹H NMR (400 MHz, CDCl₃): δ ppm 8.80-10.50 (brs, 1H), 8.13 (m, 1H), 8.00 (dd, J=7.89, 1.28 Hz, 1H), 7.10 (dd, J=7.89, 5.32 Hz, 1H), 1.69 (s, 9H).

LC-MS: Rt=0.75 min, MS ES+=236 (M+H⁺).

### Step C: Preparation of tert-butyl 2-oxo-4-(pyrimidin-5-ylmethyl)imidazo[4,5-b]pyridine-1-carboxylate

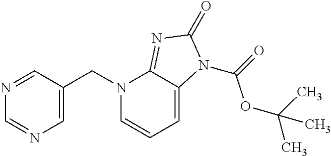 [SMILES: CC(C)(C)OC(=O)n1c2cccn(Cc3cncnc3)c-2nc1=O]

Tert-butyl 2-oxo-3H-imidazo[4,5-b]pyridine-1-carboxylate (1.000 g, 4.251 mmol) was dispersed into acetonitrile (10.63 mL). Then potassium carbonate (1.780 g, 12.75 mmol) and 5-(chloromethyl)pyrimidine hydrochloride (0.7384 g, 4.251 mmol) were added. The resulting suspension was stirred at room temperature for 4 hours, then at reflux for 17 hours and 30 minutes. The temperature was allowed to cool at room temperature and 0.5 equivalent of additional 5-(choromethyl)pyrimidine hydrochloride (0.369 g, 2.125 mmol) were added. The resulting mixture was stirred at reflux for 1 hour 30 minutes. After cooling at room temperature, the reaction medium was concentrated under reduced pressure at 40° C. and the crude material was purified by chromatography over silica gel to afford tert-butyl 2-oxo-4-(pyrimidin-5-ylmethyl)imidazo[4,5-b]pyridine-1-carboxylate.

¹H NMR (400 MHz, CDCl₃): δ ppm 9.24 (s, 1H), 8.90 (s, 2H), 7.73 (dd, J=7.33-1.1 Hz, 1H), 7.31 (dd, J=6.96-1.1 Hz, 1H), 6.74 (t, J=6.97 Hz, 1H), 5.51 (s, 2H), 1.65 (s, 9H).

LC-MS: Rₜ=0.65 min, MS ES+=328 (M+H⁺).

### Step D: Preparation of 4-(pyrimidin-5-ylmethyl)-1H-imidazo[4,5-b]pyridin-2-one

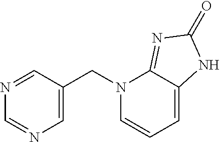 [SMILES: O=c1nc2n(Cc3cncnc3)cccc-2[nH]1]

Tert-butyl 2-oxo-4-(pyrimidin-5-ylmethyl)imidazo[4,5-b]pyridine-1-carboxylate (0.304 g, 0.929 mmol) was dissolved in dichloromethane (1.86 mL). Then trifluoroacetic acid (TFA) (0.358 mL, 4.64 mmol) was added slowly with a syringe. Gas evolution was observed. The resulting mixture was stirred at room temperature for 5 hours and then concentrated under reduced pressure at 40° C. The residue obtained was purified by chromatography over silica gel to afford 4-(pyrimidin-5-ylmethyl)-1H-imidazo[4,5-b]pyridin-2-one.

¹H NMR (400 MHz, d6-DMSO): δ ppm 10.98 (br s, 1H), 9.17 (s, 1H), 8.92 (s, 2H), 7.89 (d, J=6.24 Hz, 1H), 7.15 (d, J=6.97 Hz, 1H), 6.86 (t, J=6.97 Hz, 1H), 5.56 (s, 2H).

LC-MS: Rₜ=0.17 min, MS ES+=228 (M+H⁺).

### Step E: Synthesis of 1-phenyl-4-(pyrimidin-5-ylmethyl)imidazo[4,5-b]pyridin-2-one

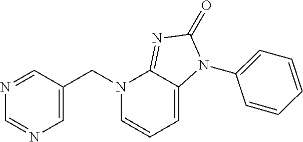 [SMILES: O=c1nc2n(Cc3cncnc3)cccc-2n1-c1ccccc1]

4-(pyrimidin-5-ylmethyl)-1H-imidazo[4,5-b]pyridin-2-one (0.200 g, 0.880 mmol) was dispersed into acetonitrile (4.40 mL). Then copper(I) iodide (0.0513 g, 0.264 mmol) and potassium carbonate (0.307 g, 2.20 mmol) were added and the resulting suspension purged with argon for 5 minutes. Then 1,2-dimethylethylenediamine (DMEDA) (0.0574 mL, 0.528 mmol) and iodobenzene (0.272 g, 0.149 mL, 1.32 mmol) were added and the resulting suspension was stirred at reflux for 5 hours and 30 minutes. After cooling at room temperature, the reaction mixture was filtered through Celite and the solid press cake was washed with MeOH (×3). The filtrate was concentrated under reduced pressure at 40° C. The crude material obtained was purified by chromatography over silica gel to afford 1-phenyl-4-(pyrimidin-5-ylmethyl)imidazo[4,5-b]pyridin-2-one.

¹H NMR (400 MHz, MeOD): δ ppm 9.15 (s, 1H), 8.99 (s, 2H), 7.92 (dd, J=6.79, 0.92 Hz, 1H), 7.50-7.60 (m, 4H), 7.43-7.47 (m, 1H), 7.29 (dd, J=7.34, 1.10 Hz, 1H), 6.97 (t, J=6.91 Hz, 1H), 5.70 (s, 2H).

LC-MS: Rₜ=0.59 min, MS ES+=304 (M+H⁺).

### Example P3: Preparation of 4-[(2-chlorothiazol-5-yl)methyl]-5-methyl-1-phenyl-imidazo[4,5-b]pyridin-2-one (Compound A90)

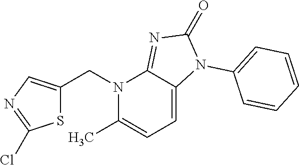 [SMILES: Cc1ccc2n(-c3ccccc3)c(=O)nc-2n1Cc1cnc(Cl)s1]

### Step A: Preparation of 5-methyl-1,3-dihydroimidazo[4,5-b]pyridin-2-one

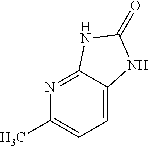

2-Amino-6-methylnicotinic acid (CAS 846021-26-9) (5.00 g, 31.9 mmol) was dispersed into 1,4-dioxane (150 mL) as a brown suspension. Then triethylamine (4.89 mL, 35.1 mmol) was added slowly followed by diphenylphosphoryl azide (7.08 mL, 31.9 mmol) added dropwise over 5 minutes. The resulting brown suspension was slowly brought to reflux, stirred at reflux for 3 hours and 30 minutes and then cooled down at room temperature. The reaction was monitored by TLC and LC-MS. The reaction medium was concentrated under reduced pressure at 40° C. to afford a brown oil. The residue obtained was purified by chromatography over silica gel to afford 5-methyl-1,3-dihydroimidazo[4,5-b]pyridin-2-one.

¹H NMR (400 MHz, d6-DMSO): δ ppm 11.13 (br s, 1H), 10.65 (br s, 1H), 7.10 (d, J=7.70 Hz, 1H), 6.78 (d, J=7.70 Hz, 1H), 2.36 (s, 3H).

LC-MS: Rₜ=0.25 min, MS ES+=150 (M+H⁺).

### Step B: Preparation of tert-butyl 5-methyl-2-oxo-3H-imidazo[4,5-b]pyridine-1-carboxylate

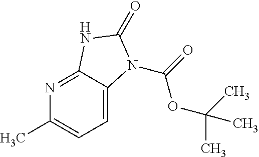 [SMILES: Cc1ccc2c(n1)[nH]c(=O)n2C(=O)OC(C)(C)C]

5-methyl-1,3-dihydroimidazo[4,5-b]pyridin-2-one (2.1411 g, 14.355 mmol) was dissolved into DMF (30 mL) and sodium hydride (0.6029 g, 15.073 mmol) added portion wise over 20 minutes at room temperature. The resulting mixture was stirred at room temperature for 1 hour. Di-tert-butyl dicarbonate (3.20 g, 14.355 mmol) dissolved in DMF (20 mL) was added dropwise over 15 minutes. The reaction mixture was vigorously stirred at room temperature for 5 hours. The reaction was monitored by TLC and LC-MS. Methanol was carefully added to quench the reaction medium and then water was added (pH=8-9). The aqueous layer was extracted once with ethyl acetate and discarded to remove impurities. Then the pH was adjusted to 7 with 4 M aq. HCl solution and the aqueous layer extracted with ethyl acetate. The combined organic layers were washed with water, dried over anhydrous sodium sulfate, filtered and concentrated under reduced pressure at 40° C. to afford tert-butyl 5-methyl-2-oxo-3H-imidazo[4,5-b]pyridine-1-carboxylate.

¹H NMR (400 MHz, CDCl₃): δ ppm 11.0-12.0 (br s, 1H), 7.87 (d, J=8.07 Hz, 1H), 6.92 (d, J=8.07 Hz, 1H), 2.66 (s, 3H), 1.68 (s, 9H).

LC-MS: Rₜ=0.81 min, MS ES+=250 (M+H⁺).

### Step C: Preparation of tert-butyl 4-[(2-chlorothiazol-5-yl)methyl]-5-methyl-2-oxo-imidazo[4,5-b]pyridine-1-carboxylate

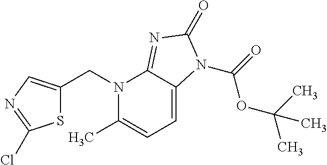 [SMILES: Cc1ccc2n(C(=O)OC(C)(C)C)c(=O)nc-2n1Cc1cnc(Cl)s1]

A 25 mL single-neck round-bottom flask was charged at room temperature with tert-butyl 5-methyl-2-oxo-3H-imidazo[4,5-b]pyridine-1-carboxylate (1.00 g, 4.01 mmol) dissolved in DMF (10.0 mL). Then potassium carbonate (1.12 g, 8.02 mmol) and 2-chloro-5-(chloromethyl)thiazole (CAS 105827-91-6) (0.674 g, 4.01 mmol) were added. The resulting yellow suspension was stirred at room temperature for 5 hours until completion of the reaction. Water was added to the reaction medium and the aqueous layer was extracted with ethyl acetate. The combined organic layers were washed with water (×2), with brine, dried over dry sodium sulfate, filtered and concentrated under reduced pressure at 40° C. The crude was purified by chromatography over silica gel to afford tert-butyl 4-[(2-chlorothiazol-5-yl)methyl]-5-methyl-2-oxo-imidazo[4,5-b]pyridine-1-carboxylate.

¹H NMR (400 MHz, CDCl₃): δ ppm 7.62 (s, 1H), 7.59 (d, J=7.70 Hz, 1H), 6.52 (d, J=7.70 Hz, 1H), 5.65 (s, 2H), 2.61 (s, 3H), 1.66 (s, 9H).

LC-MS: Rₜ=0.86 min, MS ES+=381/383 (M+H⁺).

### Step D: Preparation of 4-[(2-chlorothiazol-5-yl)methyl]-5-methyl-1H-imidazo[4,5-b]pyridin-2-one

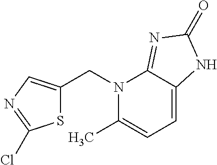 [SMILES: Cc1ccc2[nH]c(=O)nc-2n1Cc1cnc(Cl)s1]

A 5 mL single-necked round-bottom flask was charged at room temperature with tert-butyl 4-[(2-chlorothiazol-5-yl)methyl]-5-methyl-2-oxo-imidazo[4,5-b]pyridine-1-carboxylate (0.0517 g, 0.136 mmol) dissolved in dichloromethane (0.5 mL) as a light yellow solution. Then trifluoroacetic acid (0.0523 mL, 0.679 mmol) was added slowly with a syringe. The resulting mixture was stirred at room temperature for 2 hours. The reaction mixture was concentrated under reduced pressure at 40° C. to afford 4-[(2-chlorothiazol-5-yl)methyl]-5-methyl-1H-imidazo[4,5-b]pyridin-2-one.

¹H NMR (400 MHz, CDCl₃): δ ppm 7.79 (d, J=7.70 Hz, 1H), 7.69 (s, 1H), 7.17 (d, J=7.70 Hz, 1H), 5.97 (s, 2H), 2.82 (s, 3H).

LC-MS: Rₜ=0.53 min, MS ES+=281/283 (M+H⁺).

### Step E: Preparation of 4-[(2-chlorothiazol-5-yl)methyl]-5-methyl-1-phenyl-imidazo[4,5-b]pyridin-2-one

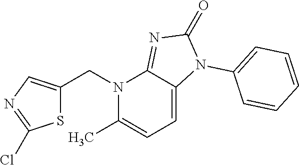 [SMILES: Cc1ccc2n(-c3ccccc3)c(=O)nc-2n1Cc1cnc(Cl)s1]

4-[(2-chlorothiazol-5-yl)methyl]-5-methyl-1H-imidazo[4,5-b]pyridin-2-one (0.070 g, 0.25 mmol) was dissolved in acetonitrile (2.5 mL), followed by the addition of copper(I) iodide (0.015 g, 0.075 mmol) and potassium carbonate (0.087 g, 0.62 mmol). The resulting yellow suspension was purged with argon for 5 minutes and DMEDA (0.016 mL, 0.15 mmol) and iodobenzene (0.042 mL, 0.37 mmol) were added. The resulting suspension was stirred at reflux for 2 hours and cooled down at room temperature. The reaction was monitored by TLC and LC-MS. After completion, water was added to the reaction medium and the aqueous layer was extracted with ethyl acetate (3×). The combined organic layers were washed with brine, dried over dry sodium sulfate, filtered and concentrated under reduced pressure at 40° C. The crude material was purified by chromatography over silica gel to afford 4-[(2-chlorothiazol-5-yl)methyl]-5-methyl-1-phenyl-imidazo[4,5-b]pyridin-2-one.

¹H NMR (400 MHz, CDCl₃): δ ppm 7.69 (s, 1H), 7.44-7.53 (m, 4H), 7.30-7.37 (m, 1H), 6.99 (d, J=7.70 Hz, 1H), 6.52 (d, J=7.70 Hz, 1H), 5.77 (s, 2H), 2.65 (s, 3H).

LC-MS: Rₜ=0.82 min, MS ES+=357/359 (M+H⁺).

### Example P4: Preparation of 4-[(2-chlorothiazol-5-yl)methyl]-1-phenyl-imidazo[4,5-b]pyridin-2-one (Compound A36)

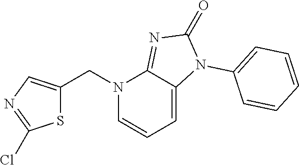 [SMILES: O=c1nc2n(Cc3cnc(Cl)s3)cccc-2n1-c1ccccc1]

### Step A: Preparation of tert-butyl 4-[(2-chlorothiazol-5-yl)methyl]-2-oxo-imidazo[4,5-b]pyridine-1-carboxylate

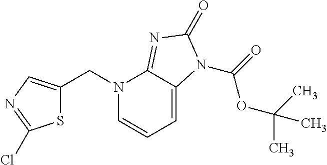 [SMILES: CC(C)(C)OC(=O)n1c2cccn(Cc3cnc(Cl)s3)c-2nc1=O]

Tert-butyl 2-oxo-3H-imidazo[4,5-b]pyridine-1-carboxylate (1.00 g, 4.25 mmol) was dispersed into acetonitrile (21.3 mL). Potassium carbonate (1.19 g, 8.50 mmol) and 2-chloro-5-(chloromethyl)thiazole (CAS 105827-91-6) (0.714 g, 4.25 mmol) were then added. The resulting suspension was stirred at room temperature for 19 hours, then at reflux for 2 hours and 30 minutes. After cooling at room temperature, water was added to the reaction medium and the aqueous layer was extracted with ethyl acetate. The combined organic layers were washed with brine, dried over dry sodium sulfate, filtered and evaporated. The crude material was purified by chromatography.

¹H NMR (400 MHz, CDCl₃): δ ppm 7.70 (dd, J=7.52, 0.92 Hz, 1H), 7.67-7.69 (m, 1H), 7.27-7.30 (m, 1H), 6.72 (t, J=6.95 Hz, 1H), 5.54-5.57 (m, 2H), 1.66 (s, 9H).

LC-MS: Rt=0.81 min. MS ES+=367/369 (M+H⁺).

### Step B: Preparation of 4-[(2-chlorothiazol-5-yl)methyl]-1H-imidazo[4,5-b]pyridin-2-one

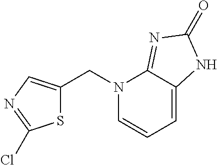 [SMILES: O=c1nc2n(Cc3cnc(Cl)s3)cccc-2[nH]1]

Tert-butyl 4-[(2-chlorothiazol-5-yl)methyl]-2-oxo-imidazo[4,5-b]pyridine-1-carboxylate (0.3340 g, 0.9106 mmol) was dissolved in dichloromethane (1 mL) and then TFA (0.3508 mL, 4.553 mmol) was added slowly with a syringe. The resulting mixture was stirred at room temperature for 5 hours. The reaction mixture was concentrated under reduced pressure at 40° C. and the crude material was purified by chromatography over silica gel to afford 4-[(2-chlorothiazol-5-yl)methyl]-1H-imidazo[4,5-b]pyridin-2-one.

¹H NMR (400 MHz, d6-DMSO): δ ppm 10.84 (br s, 1H), 7.86 (s, 1H), 7.76 (d, J=6.24 Hz, 1H), 7.05 (d, J=6.97 Hz, 1H), 6.76 (t, J=6.97 Hz, 1H), 5.65 (s, 2H).

LC-MS: Rt=0.37 min, MS ES+=267/269 (M+H⁺).

### Step C: Preparation of 4-[(2-chlorothiazol-5-yl)methyl]-1-phenyl-imidazo[4,5-b]pyridin-2-one

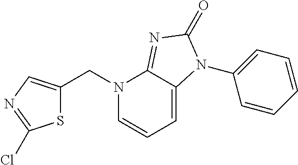 [SMILES: O=c1nc2n(Cc3cnc(Cl)s3)cccc-2n1-c1ccccc1]

Under argon, 4-[(2-chlorothiazol-5-yl)methyl]-1H-imidazo[4,5-b]pyridin-2-one (0.070 g, 0.26 mmol,) was dispersed into acetonitrile (4 mL) followed by the addition of copper(I) iodide (0.015 g, 0.079 mmol) and potassium carbonate (0.092 g, 0.66 mmol) were added. The resulting beige suspension was purged with argon for 5 minutes. Then DMEDA (0.017 mL, 0.16 mmol) and iodobenzene (0.044 mL, 0.39 mmol) were added and the resulting suspension was stirred at reflux for 1 hour and 30 minutes. After cooling at room temperature, water was added to the reaction medium and extraction was carried out with ethyl acetate (×3). The combined organic layers were washed with brine, dried over dry sodium sulfate, filtered and concentrated under reduced pressure at 40° C. The crude material was purified by chromatography over silica gel to afford 4-[(2-chlorothiazol-5-yl)methyl]-1-phenyl-imidazo[4,5-b]pyridin-2-one.

¹H NMR (400 MHz, CDCl₃): δ ppm 7.73 (s, 1H), 7.48-7.56 (m, 4H), 7.34-7.42 (m, 1H), 7.27 (d, 1H), 7.02 (d, 1H), 6.68 (t, 1H), 5.64 (s, 2H).

LC-MS: Rt=0.77 min, MS ES+=343/345 (M+H⁺).

### Example P5: Preparation of Compounds A41 to A80

To a solution of 4-[(2-chlorothiazol-5-yl)methyl]-1H-imidazo[4,5-b]pyridin-2-one (prepared according to example P4, Step B) (0.03 mmol in 1 mL N,N-dimethylformamide (DMF)) was added potassium carbonate K₂CO₃ (3.0 eq.), copper (I) iodide (0.3 eq.), trans-N,N′-dimethylcyclohexane-1,2-diamine (1.0 eq.) and the appropriate aryl iodide or bromide (1 eq.). The mixture was submitted to microwave radiation for 15 minutes at 140° C. The DMF was evaporated and the residue dissolved in ethyl acetate. A solution of EDTA (12% in water) was added and the water phase extracted three times with ethyl acetate. The organic phases were combined and evaporated under vacuum. The desired compounds were isolated by HPLC and identified by LC-MS (method B).

The compounds A1 to A102 may be prepared by analogy with the reactions described at Examples P1 to P5.

TABLE A
This table discloses compounds of the formula (I):

| | | | ¹HNMR or LC-MS | | |
|---|---|---|---|---|---|
| | | | Rt | [M + H] | |
| Entry | IUPAC name | Structure | (min) | (measured) | Method |
| A1 | 4-[(6-chloro-3- pyridyl)methyl]-1-(3- phenylphenyl) imidazo[4,5-b]pyridin-2- one | 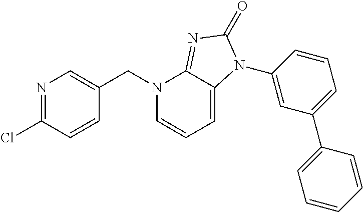 [SMILES: O=c1nc2n(Cc3ccc(Cl)nc3)cccc-2n1-c1cccc(-c2ccccc2)c1] | 0.93 | 413 | A |
| A2 | 4-[(6-chloro-3- pyridyl)methyl]-1-(1- methylpyrazol-3- yl)imidazo[4,5-b]pyridin- 2-one | 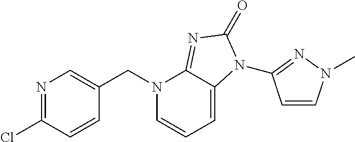 [SMILES: Cn1ccc(-n2c3cccn(Cc4ccc(Cl)nc4)c-3nc2=O)n1] | 0.67 | 341 | A |
| A3 | 4-[(6-chloro-3- pyridyl)methyl]-1-(1- methylpyrazol-4- yl)imidazo[4,5-b]pyridin- 2-one | 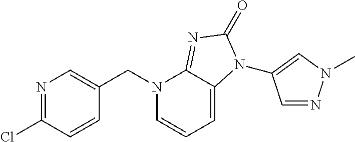 [SMILES: Cn1cc(-n2c3cccn(Cc4ccc(Cl)nc4)c-3nc2=O)cn1] | 0.6 | 341 | A |
| A4 | 4-[(6-chloro-3- pyridyl)methyl]-1-(1- phenylpyrazol-3- yl)imidazo[4,5-b]pyridin- 2-one | 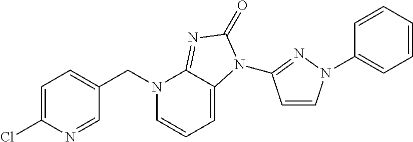 [SMILES: O=c1nc2n(Cc3ccc(Cl)nc3)cccc-2n1-c1ccn(-c2ccccc2)n1] | 0.82 | 403 | A |
| A5 | 4-[(6-chloro-3- pyridyl)methyl]-1-(1- phenylpyrazol-4- yl)imidazo[4,5-b]pyridin- 2-one | 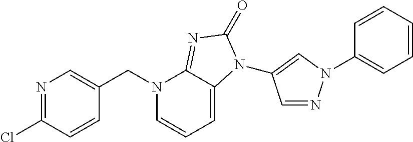 [SMILES: O=c1nc2n(Cc3ccc(Cl)nc3)cccc-2n1-c1cnn(-c2ccccc2)c1] | 0.9 | 403 | A |
| A6 | 1-(3-chlorophenyl)-4-[(6- chloro-3- pyridyl)methyl]imidazo [4,5-b]pyridin-2-one | 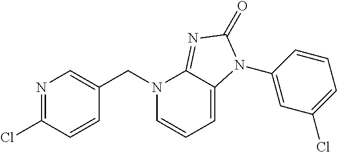 [SMILES: O=c1nc2n(Cc3ccc(Cl)nc3)cccc-2n1-c1cccc(Cl)c1] | 0.83 | 371 | A |
| A7 | 4-[(6-chloro-3- pyridyl)methyl]-1-(3- methoxyphenyl)imidazo [4,5-b]pyridin-2-one | 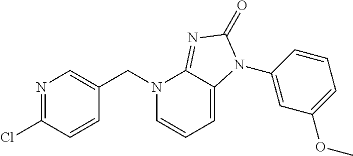 [SMILES: COc1cccc(-n2c3cccn(Cc4ccc(Cl)nc4)c-3nc2=O)c1] | 0.77 | 367 | A |
| A8 | 4-[(6-chloro-3- pyridyl)methyl]-1-(4- methoxyphenyl)imidazo [4,5-b]pyridin-2-one | 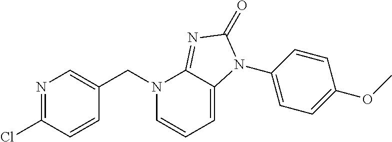 [SMILES: COc1ccc(-n2c3cccn(Cc4ccc(Cl)nc4)c-3nc2=O)cc1] | 0.76 | 367 | A |
| A9 | 1-(4-chlorophenyl)-4-[(6- chloro-3- pyridyl)methyl]imidazo [4,5-b]pyridin-2-one | 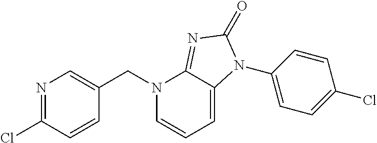 [SMILES: O=c1nc2n(Cc3ccc(Cl)nc3)cccc-2n1-c1ccc(Cl)cc1] | 0.83 | 371 | A |
| A10 | 4-[4-[(6-chloro-3- pyridyl)methyl]-2-oxo- imidazo[4,5-b]pyridin-1- yl]benzonitrile | 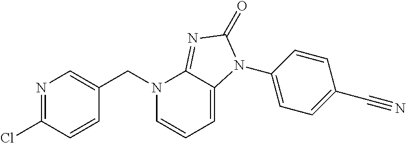 [SMILES: N#Cc1ccc(-n2c3cccn(Cc4ccc(Cl)nc4)c-3nc2=O)cc1] | 0.72 | 362 | A |
| A11 | 3-[4-[(6-chloro-3- pyridyl)methyl]-2-oxo- imidazo[4,5-b]pyridin-1- yl]benzonitrile | 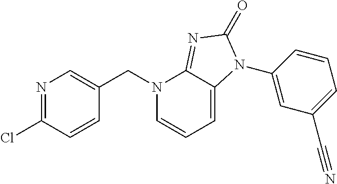 [SMILES: N#Cc1cccc(-n2c3cccn(Cc4ccc(Cl)nc4)c-3nc2=O)c1] | 0.73 | 362 | A |
| A12 | 4-[(6-chloro-3- pyridyl)methyl]-1-[5- (trifluoromethyl)-2- pyridyl]imidazo[4,5- b]pyridin-2-one | 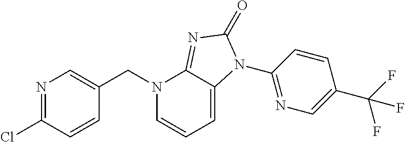 [SMILES: O=c1nc2n(Cc3ccc(Cl)nc3)cccc-2n1-c1ccc(C(F)(F)F)cn1] | 0.92 | 406 | A |
| A13 | 4-[(6-chloro-3- pyridyl)methyl]-1-[4- (trifluoromethyl)phenyl] imidazo[4,5-b]pyridin-2- one | 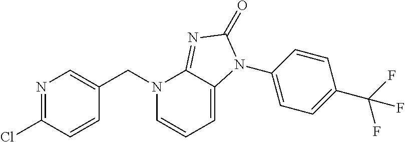 [SMILES: O=c1nc2n(Cc3ccc(Cl)nc3)cccc-2n1-c1ccc(C(F)(F)F)cc1] | 0.87 | 405 | A |
| A14 | 4-[(6-chloro-3- pyridyl)methyl]-1-[2- (trifluoromethyl)-4- pyridyl]imidazo[4,5- b]pyridin-2-one | 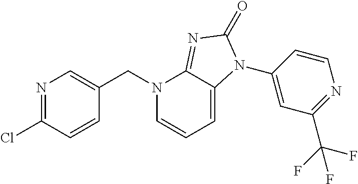 [SMILES: O=c1nc2n(Cc3ccc(Cl)nc3)cccc-2n1-c1ccnc(C(F)(F)F)c1] | 0.78 | 406 | A |
| A15 | 4-[(6-chloro-3- pyridyl)methyl]-1-(4- phenylphenyl)imidazo [4,5-b]pyridin-2-one | 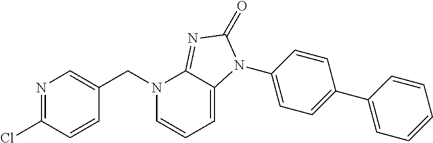 [SMILES: O=c1nc2n(Cc3ccc(Cl)nc3)cccc-2n1-c1ccc(-c2ccccc2)cc1] | 0.94 | 413 | A |
| A16 | 4-[(6-chloro-3- pyridyl)methyl]-1- pyrimidin-5-yl- imidazo[4,5-b]pyridin-2- one | 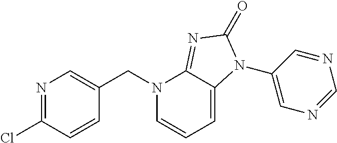 [SMILES: O=c1nc2n(Cc3ccc(Cl)nc3)cccc-2n1-c1cncnc1] | 0.57 | 339 | A |
| A17 | 4-[(6-chloro-3- pyridyl)methyl]-1-[6- (trifluoromethyl)pyridazin- 3-yl]imidazo[4,5- b]pyridin-2-one | 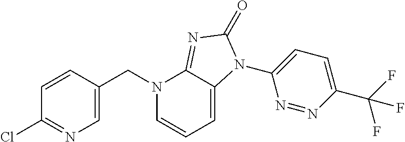 [SMILES: O=c1nc2n(Cc3ccc(Cl)nc3)cccc-2n1-c1ccc(C(F)(F)F)nn1] | 0.86 | 407 | A |
| A18 | 4-[(6-chloro-3- pyridyl)methyl]-1- pyrazin-2-yl-imidazo[4,5- b]pyridin-2-one | 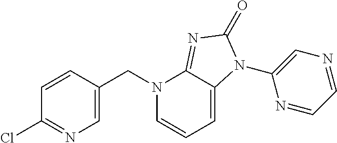 [SMILES: O=c1nc2n(Cc3ccc(Cl)nc3)cccc-2n1-c1cnccn1] | 0.67 | 339 | A |
| A19 | 4-[(6-chloro-3- pyridyl)methyl]-1-[4- (difluoromethyl)-2- pyridyl]imidazo[4,5- b]pyridin-2-one | 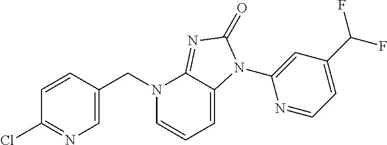 [SMILES: O=c1nc2n(Cc3ccc(Cl)nc3)cccc-2n1-c1cc(C(F)F)ccn1] | 0.83 | 388 | A |
| A20 | 1-(5-chloropyrazin-2-yl)- 4-[(6-chloro-3- pyridyl)methyl]imidazo [4,5-b]pyridin-2-one | 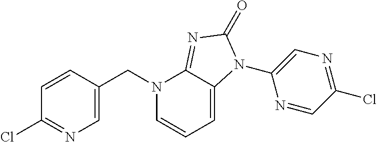 [SMILES: O=c1nc2n(Cc3ccc(Cl)nc3)cccc-2n1-c1cnc(Cl)cn1] | 0.82 | 373 | A |
| A21 | 4-[(6-chloro-3- pyridyl)methyl]-1-[3- (trifluoromethyl)phenyl] imidazo[4,5-b]pyridin-2- one | 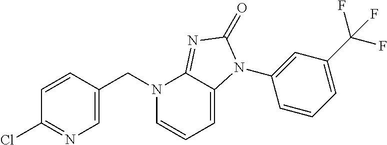 [SMILES: O=c1nc2n(Cc3ccc(Cl)nc3)cccc-2n1-c1cccc(C(F)(F)F)c1] | 0.88 | 405 | A |
| A22 | 1-(5-chloro-3-pyridyl)-4- [(6-chloro-3- pyridyl)methyl]imidazo [4,5-b]pyridin-2-one | 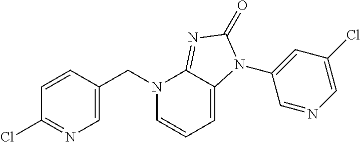 [SMILES: O=c1nc2n(Cc3ccc(Cl)nc3)cccc-2n1-c1cncc(Cl)c1] | 0.73 | 374 | A |
| A23 | 4-[(6-chloro-3- pyridyl)methyl]-1- pyrimidin-2-yl- imidazo[4,5-b]pyridin-2- one | 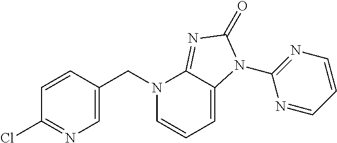 [SMILES: O=c1nc2n(Cc3ccc(Cl)nc3)cccc-2n1-c1ncccn1] | 0.61 | 339 | A |
| A24 | 4-[(6-chloro-3- pyridyl)methyl]-1-[6- (trifluoromethyl)-3- pyridyl]imidazo[4,5- b]pyridin-2-one | 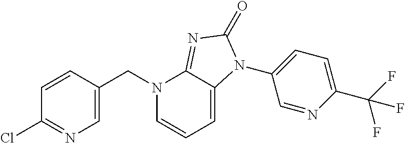 [SMILES: O=c1nc2n(Cc3ccc(Cl)nc3)cccc-2n1-c1ccc(C(F)(F)F)nc1] | 0.81 | 406 | A |
| A25 | 4-[(6-chloro-3- pyridyl)methyl]-1-[5- (trifluoromethyl)-3- pyridyl]imidazo[4,5- b]pyridin-2-one | 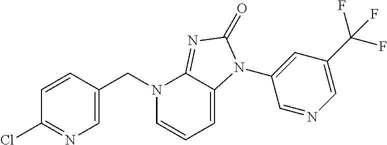 [SMILES: O=c1nc2n(Cc3ccc(Cl)nc3)cccc-2n1-c1cncc(C(F)(F)F)c1] | 0.79 | 406 | A |
| A26 | 4-[(6-chloro-3- pyridyl)methyl]-1-(5- fluoro-3- pyridyl)imidazo[4,5- b]pyridin-2-one | 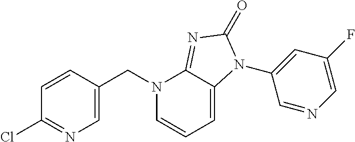 [SMILES: O=c1nc2n(Cc3ccc(Cl)nc3)cccc-2n1-c1cncc(F)c1] | 0.67 | 356 | A |
| A27 | 4-[(6-chloro-3- pyridyl)methyl]-1-(2- pyridyl)imidazo[4,5- b]pyridin-2-one | 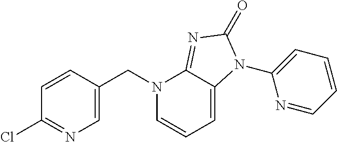 [SMILES: O=c1nc2n(Cc3ccc(Cl)nc3)cccc-2n1-c1ccccn1] | 0.73 | 338 | A |
| A28 | 4-[(6-chloro-3- pyridyl)methyl]-1-(4- pyridyl)imidazo[4,5- b]pyridin-2-one | 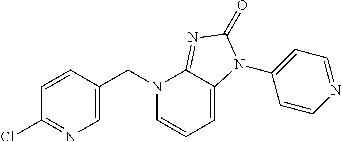 [SMILES: O=c1nc2n(Cc3ccc(Cl)nc3)cccc-2n1-c1ccncc1] | 0.4 | 338 | A |
| A29 | 4-[(6-chloro-3- pyridyl)methyl]-1-(m- tolyl)imidazo[4,5- b]pyridin-2-one | 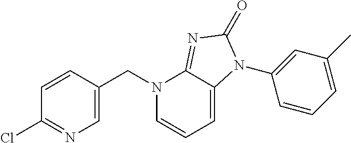 [SMILES: Cc1cccc(-n2c3cccn(Cc4ccc(Cl)nc4)c-3nc2=O)c1] | 0.82 | 352 | A |
| A30 | 4-[(6-chloro-3- pyridyl)methyl]-1-(p- tolyl)imidazo[4,5- b]pyridin-2-one | 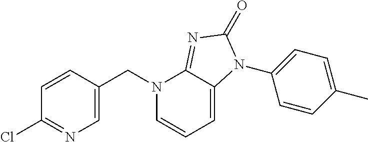 [SMILES: Cc1ccc(-n2c3cccn(Cc4ccc(Cl)nc4)c-3nc2=O)cc1] | 0.82 | 352 | A |
| A31 | 4-[(6-chloro-3- pyridyl)methyl]-1-(3- fluorophenyl)imidazo[4,5- b]pyridin-2-one | 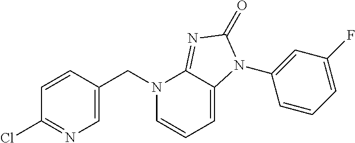 [SMILES: O=c1nc2n(Cc3ccc(Cl)nc3)cccc-2n1-c1cccc(F)c1] | 0.78 | 355 | A |
| A32 | 4-[(6-chloro-3- pyridyl)methyl]-1-(4- fluorophenyl)imidazo[4,5- b]pyridin-2-one | 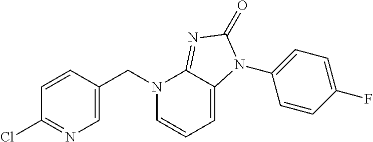 [SMILES: O=c1nc2n(Cc3ccc(Cl)nc3)cccc-2n1-c1ccc(F)cc1] | 0.78 | 355 | A |
| A33 | 4-[(6-chloro-3- pyridyl)methyl]-1-(3- pyridyl)imidazo[4,5- b]pyridin-2-one | 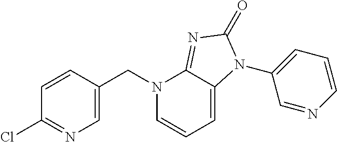 [SMILES: O=c1nc2n(Cc3ccc(Cl)nc3)cccc-2n1-c1cccnc1] | 0.59 | 338 | A |
| A34 | 4-[(6-chloro-3- pyridyl)methyl]-1-phenyl- imidazo[4,5-b]pyridin-2- one | 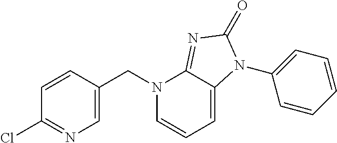 [SMILES: O=c1nc2n(Cc3ccc(Cl)nc3)cccc-2n1-c1ccccc1] | 0.76 | 336 | A |
| A35 | 1-phenyl-4-(pyrimidin-5- ylmethyl)imidazo[4,5- b]pyridin-2-one | 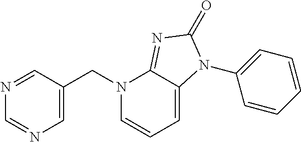 [SMILES: O=c1nc2n(Cc3cncnc3)cccc-2n1-c1ccccc1] | 0.59 | 304 | A |
| A36 | 4-[(2-chlorothiazol-5- yl)methyl]-1-phenyl- imidazo[4,5-b]pyridin-2- one | 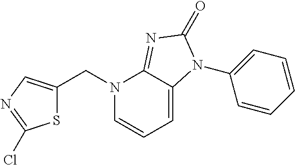 [SMILES: O=c1nc2n(Cc3cnc(Cl)s3)cccc-2n1-c1ccccc1] | 0.76 | 343 | A |
| A37 | 4-[(6-chloro-3- pyridyl)methyl]-1- pyrimidin-4-yl- imidazo[4,5-b]pyridin-2- one | 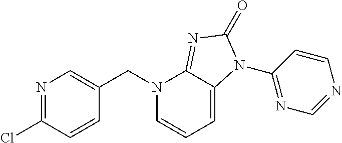 [SMILES: O=c1nc2n(Cc3ccc(Cl)nc3)cccc-2n1-c1ccncn1] | 0.68 | 339 | A |
| A38 | 1-(2-chlorophenyl)-4-[(6- chloro-3- pyridyl)methyl]imidazo [4,5-b]pyridin-2-one | 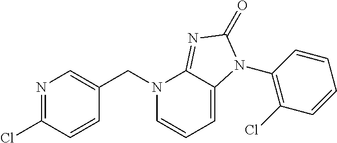 [SMILES: O=c1nc2n(Cc3ccc(Cl)nc3)cccc-2n1-c1ccccc1Cl] | 0.79 | 371 | A |
| A39 | 4-[(6-chloro-3- pyridyl)methyl]-1-[4- (trifluoromethoxy)phenyl] imidazo[4,5-b]pyridin-2- one | 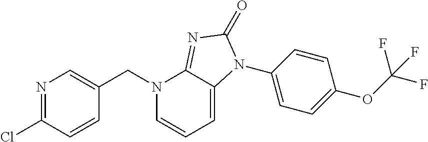 [SMILES: O=c1nc2n(Cc3ccc(Cl)nc3)cccc-2n1-c1ccc(OC(F)(F)F)cc1] | 0.9 | 421 | A |
| A40 | 2-[4-[(6-chloro-3- pyridyl)methyl]-2-oxo- imidazo[4,5-b]pyridin-1- yl]benzonitrile | 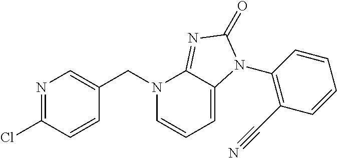 [SMILES: N#Cc1ccccc1-n1c2cccn(Cc3ccc(Cl)nc3)c-2nc1=O] | 0.71 | 362 | A |
| A41 | 4-[(6-chloro-3- pyridyl)methyl]-1-(2,4- dichlorophenyl)imidazo [4,5-b]pyridin-2-one | 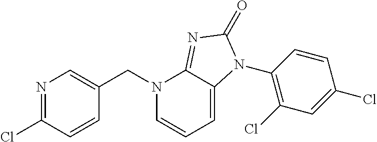 [SMILES: O=c1nc2n(Cc3ccc(Cl)nc3)cccc-2n1-c1ccc(Cl)cc1Cl] | 0.87 | 405 | A |
| A42 | 4-[(6-chloro-3- pyridyl)methyl]-1-[3- (trifluoromethyl)pyrazin- 2-yl]imidazo[4,5- b]pyridin-2-one | 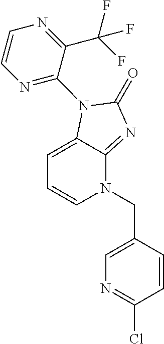 [SMILES: O=c1nc2n(Cc3ccc(Cl)nc3)cccc-2n1-c1nccnc1C(F)(F)F] | 0.98 | 407.04 | B |
| A43 | 4-[(6-chloro-3- pyridyl)methyl]-1-(4- methyl-2- pyridyl)imidazo[4,5- b]pyridin-2-one | 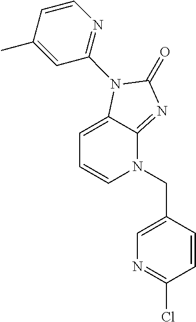 [SMILES: Cc1ccnc(-n2c3cccn(Cc4ccc(Cl)nc4)c-3nc2=O)c1] | 1.11 | 352.04 | B |
| A44 | 4-[(6-chloro-3- pyridyl)methyl]-1-(6- methoxy-2- pyridyl)imidazo[4,5- b]pyridin-2-one | 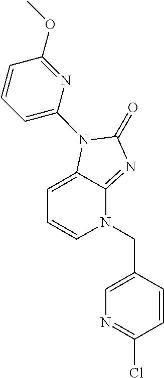 [SMILES: COc1cccc(-n2c3cccn(Cc4ccc(Cl)nc4)c-3nc2=O)n1] | 1.19 | 368.05 | B |
| A45 | 4-[(6-chloro-3- pyridyl)methyl]-1-[5- (trifluoromethyl)pyrazin- 2-yl]imidazo[4,5- b]pyridin-2-one | 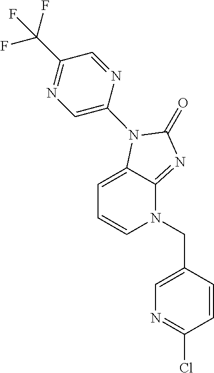 [SMILES: O=c1nc2n(Cc3ccc(Cl)nc3)cccc-2n1-c1cnc(C(F)(F)F)cn1] | 1.33 | 407.03 | B |
| A46 | ethyl 3-[4-[(6-chloro-3- pyridyl)methyl]-2-oxo- imidazo[4,5-b]pyridin-1- yl]benzoate | 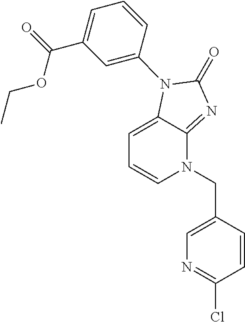 [SMILES: CCOC(=O)c1cccc(-n2c3cccn(Cc4ccc(Cl)nc4)c-3nc2=O)c1] | 1.23 | 409.10 | B |
| A47 | 4-[(6-chloro-3- pyridyl)methyl]-1-(3- thienyl)imidazo[4,5- b]pyridin-2-one | 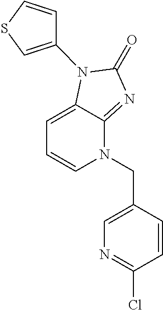 [SMILES: O=c1nc2n(Cc3ccc(Cl)nc3)cccc-2n1-c1ccsc1] | 1.01 | 342.99 | B |
| A48 | 1-(3-acetylphenyl)-4-[(6- chloro-3- pyridyl)methyl]imidazo [4,5-b]pyridin-2-one | 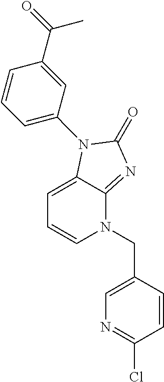 [SMILES: CC(=O)c1cccc(-n2c3cccn(Cc4ccc(Cl)nc4)c-3nc2=O)c1] | 1.01 | 379.06 | B |
| A49 | 1-(5-aminopyrazin-2-yl)- 4-[(6-chloro-3- pyridyl)methyl]imidazo [4,5-b]pyridin-2-one | 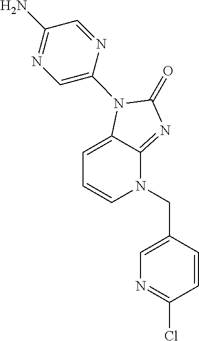 [SMILES: Nc1cnc(-n2c3cccn(Cc4ccc(Cl)nc4)c-3nc2=O)cn1] | 0.74 | 354.03 | B |
| A50 | 4-[(6-chloro-3- pyridyl)methyl]-1-(3- ethyl-4-iodo- phenyl)imidazo[4,5- b]pyridin-2-one | 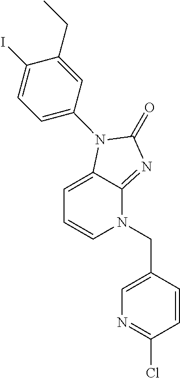 [SMILES: CCc1cc(-n2c3cccn(Cc4ccc(Cl)nc4)c-3nc2=O)ccc1I] | 1.53 | 491.06 | B |
| A51 | 4-[(6-chloro-3- pyridyl)methyl]-1-(2,5- dimethylphenyl)imidazo [4,5-b]pyridin-2-one | 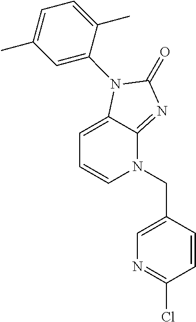 [SMILES: Cc1ccc(C)c(-n2c3cccn(Cc4ccc(Cl)nc4)c-3nc2=O)c1] | 1.23 | 365.07 | B |
| A52 | 2-[3-[4-[(6-chloro-3- pyridyl)methyl]-2-oxo- imidazo[4,5-b]pyridin-1- yl]phenyl]acetonitrile | 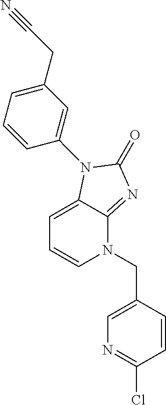 [SMILES: N#CCc1cccc(-n2c3cccn(Cc4ccc(Cl)nc4)c-3nc2=O)c1] | 0.99 | 376.05 | B |
| A53 | 4-[(6-chloro-3- pyridyl)methyl]-1-(2- fluoro-4- pyridyl)imidazo[4,5- b]pyridin-2-one | 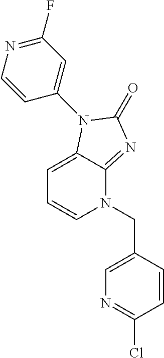 [SMILES: O=c1nc2n(Cc3ccc(Cl)nc3)cccc-2n1-c1ccnc(F)c1] | 0.93 | 356.02 | B |
| A54 | 4-[(6-chloro-3- pyridyl)methyl]-1-(5- nitro-2- pyridyl)imidazo[4,5- b]pyridin-2-one | 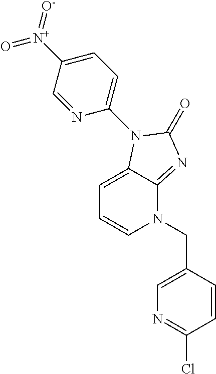 | 1.21 | 383.04 | B |
| A55 | 4-[(6-chloro-3- pyridyl)methyl]-1-(6- methyl-2- pyridyl)imidazo[4,5- b]pyridin-2-one | 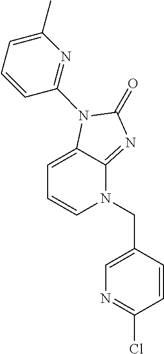 [SMILES: Cc1cccc(-n2c3cccn(Cc4ccc(Cl)nc4)c-3nc2=O)n1] | 1.13 | 352.04 | B |
| A56 | 4-[(6-chloro-3- pyridyl)methyl]-1-[4- (trifluoromethyl)-2- pyridyl]imidazo[4,5- b]pyridin-2-one | 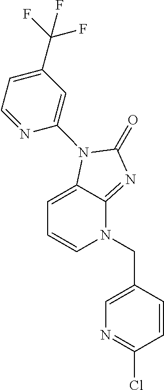 [SMILES: O=c1nc2n(Cc3ccc(Cl)nc3)cccc-2n1-c1cc(C(F)(F)F)ccn1] | 1.38 | 406.04 | B |
| A57 | ethyl 5-[4-[(6-chloro-3- pyridyl)methyl]-2-oxo- imidazo[4,5-b]pyridin-1- yl]thiophene-2- carboxylate | 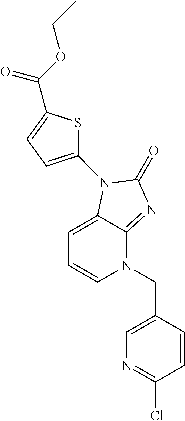 [SMILES: CCOC(=O)c1ccc(-n2c3cccn(Cc4ccc(Cl)nc4)c-3nc2=O)s1] | 1.27 | 415.05 | B |
| A58 | 4-[(6-chloro-3- pyridyl)methyl]-1-(4- methyl-3- thienyl)imidazo[4,5- b]pyridin-2-one | 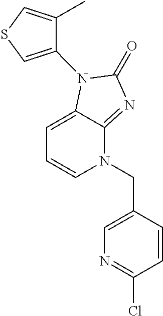 [SMILES: Cc1cscc1-n1c2cccn(Cc3ccc(Cl)nc3)c-2nc1=O] | 1.06 | 357.00 | B |
| A59 | 4-[(6-chloro-3- pyridyl)methyl]-1-[5- (2,2,2-trifluoroacetyl)-2- thienyl]imidazo[4,5- b]pyridin-2-one | 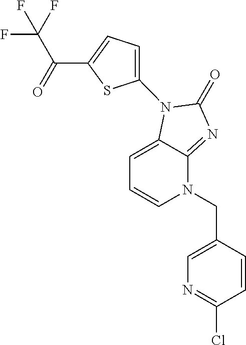 [SMILES: O=C(c1ccc(-n2c3cccn(Cc4ccc(Cl)nc4)c-3nc2=O)s1)C(F)(F)F] | 1.40 | 439.02 | B |
| A60 | 4-[(6-chloro-3- pyridyl)methyl]-1-(5- chlorothiazol-2- yl)imidazo[4,5-b]pyridin- 2-one | 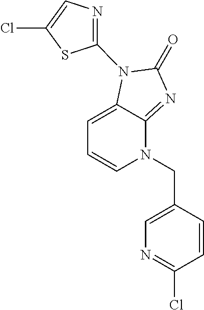 [SMILES: O=c1nc2n(Cc3ccc(Cl)nc3)cccc-2n1-c1ncc(Cl)s1] | 1.40 | 377.96 | B |
| A61 | 4-[(6-chloro-3- pyridyl)methyl]-1-[5- (trifluoromethyl)-1,3,4- thiadiazol-2- yl]imidazo[4,5-b]pyridin- 2-one | 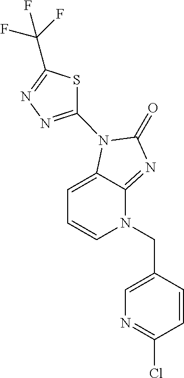 [SMILES: O=c1nc2n(Cc3ccc(Cl)nc3)cccc-2n1-c1nnc(C(F)(F)F)s1] | 1.39 | 413.03 | B |
| A62 | 4-[(6-chloro-3- pyridyl)methyl]-1-(1- isopropylpyrazol-4- yl)imidazo[4,5-b]pyridin- 2-one | 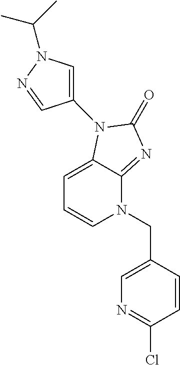 [SMILES: CC(C)n1cc(-n2c3cccn(Cc4ccc(Cl)nc4)c-3nc2=O)cn1] | 0.97 | 369.08 | B |
| A63 | 4-[(6-chloro-3- pyridyl)methyl]-1-(1- ethylpyrazol-4- yl)imidazo[4,5-b]pyridin- 2-one | 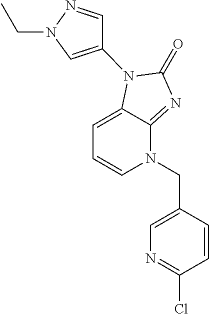 [SMILES: CCn1cc(-n2c3cccn(Cc4ccc(Cl)nc4)c-3nc2=O)cn1] | 0.87 | 355.06 | B |
| A64 | 5-[4-[(6-chloro-3- pyridyl)methyl]-2-oxo- imidazo[4,5-b]pyridin-1- yl]thiophene-2- carbonitrile | 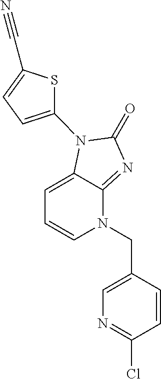 [SMILES: N#Cc1ccc(-n2c3cccn(Cc4ccc(Cl)nc4)c-3nc2=O)s1] | 1.09 | 367.98 | B |
| A65 | 5-[4-[(6-chloro-3- pyridyl)methyl]-2-oxo- imidazo[4,5-b]pyridin-1- yl]thiophene-3- carbonitrile | 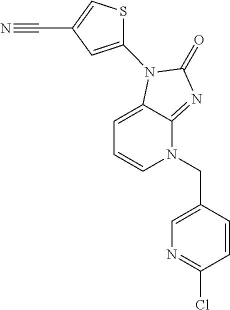 [SMILES: N#Cc1csc(-n2c3cccn(Cc4ccc(Cl)nc4)c-3nc2=O)c1] | 1.03 | 367.98 | B |
| A66 | 4-[(6-chloro-3- pyridyl)methyl]-1-thiazol- 5-yl-imidazo[4,5- b]pyridin-2-one | 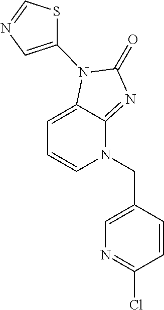 [SMILES: O=c1nc2n(Cc3ccc(Cl)nc3)cccc-2n1-c1cncs1] | 0.82 | 343.99 | B |
| A67 | 4-[(6-chloro-3- pyridyl)methyl]-1-(5- methyl-2- thienyl)imidazo[4,5- b]pyridin-2-one | 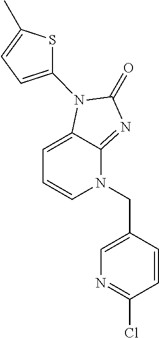 [SMILES: Cc1ccc(-n2c3cccn(Cc4ccc(Cl)nc4)c-3nc2=O)s1] | 1.17 | 357.01 | B |
| A68 | 4-[(6-chloro-3- pyridyl)methyl]-1-[5- (difluoromethyl)-3- thienyl]imidazo[4,5- b]pyridin-2-one | 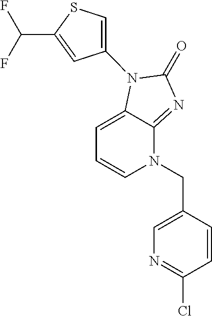 [SMILES: O=c1nc2n(Cc3ccc(Cl)nc3)cccc-2n1-c1csc(C(F)F)c1] | 1.16 | 393.02 | B |
| A69 | 4-[(6-chloro-3- pyridyl)methyl]-1-[4- (trifluoromethyl)oxazol- 2-yl]imidazo[4,5- b]pyridin-2-one | 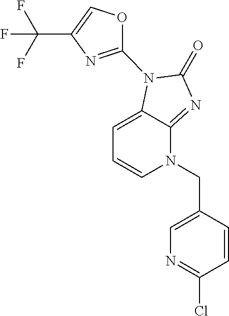 [SMILES: O=c1nc2n(Cc3ccc(Cl)nc3)cccc-2n1-c1nc(C(F)(F)F)co1] | 1.19 | 396.04 | B |
| A70 | 4-[(6-chloro-3- pyridyl)methyl]-1-(3- furyl)imidazo[4,5- b]pyridin-2-one | 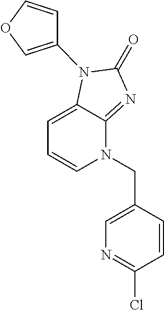 [SMILES: O=c1nc2n(Cc3ccc(Cl)nc3)cccc-2n1-c1ccoc1] | 0.93 | 327.00 | B |
| A71 | 4-[(6-chloro-3- pyridyl)methyl]-1-[5- (trifluoromethyl)-3- thienyl]imidazo[4,5- b]pyridin-2-one | 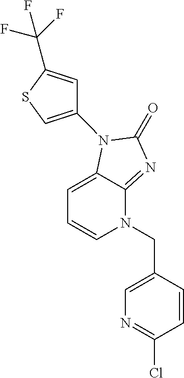 [SMILES: O=c1nc2n(Cc3ccc(Cl)nc3)cccc-2n1-c1csc(C(F)(F)F)c1] | 1.32 | 411.02 | B |
| A72 | 4-[(6-chloro-3- pyridyl)methyl]-1-[5- (difluoromethyl)-2- thienyl]imidazo[4,5- b]pyridin-2-one | 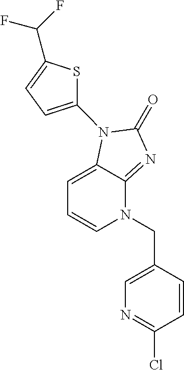 [SMILES: O=c1nc2n(Cc3ccc(Cl)nc3)cccc-2n1-c1ccc(C(F)F)s1] | 1.19 | 393.02 | B |
| A73 | 4-[(6-chloro-3- pyridyl)methyl]-1-(4- fluoro-2- thienyl)imidazo[4,5- b]pyridin-2-one | 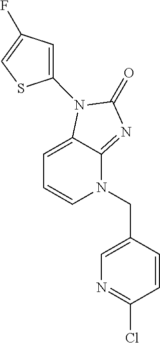 [SMILES: O=c1nc2n(Cc3ccc(Cl)nc3)cccc-2n1-c1cc(F)cs1] | 1.11 | 360.99 | B |
| A74 | 4-[(6-chloro-3- pyridyl)methyl]-1-[2- (trifluoromethyl)thiazol- 4-yl]imidazo[4,5- b]pyridin-2-one | 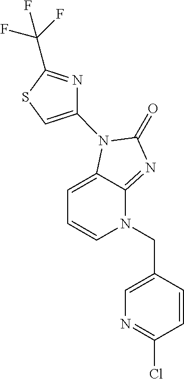 [SMILES: O=c1nc2n(Cc3ccc(Cl)nc3)cccc-2n1-c1csc(C(F)(F)F)n1] | 1.40 | 412.01 | B |
| A75 | 4-[4-[(6-chloro-3- pyridyl)methyl]-2-oxo- imidazo[4,5-b]pyridin-1- yl]thiophene-2- carbonitrile | 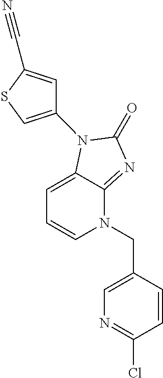 [SMILES: N#Cc1cc(-n2c3cccn(Cc4ccc(Cl)nc4)c-3nc2=O)cs1] | 1.01 | 367.99 | B |
| A76 | 4-[(6-chloro-3- pyridyl)methyl]-1-(5- methylthiazol-2- yl)imidazo[4,5-b]pyridin- 2-one | 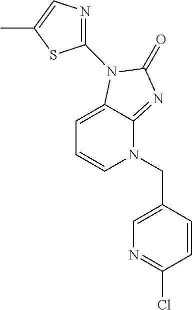 [SMILES: Cc1cnc(-n2c3cccn(Cc4ccc(Cl)nc4)c-3nc2=O)s1] | 1.24 | 358.00 | B |
| A77 | methyl 5-[4-[(6-chloro-3- pyridyl)methyl]-2-oxo- imidazo[4,5-b]pyridin-1- yl]furan-2-carboxylate | 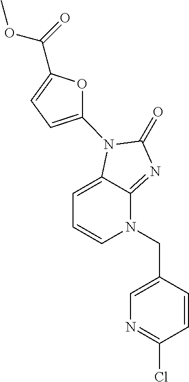 [SMILES: COC(=O)c1ccc(-n2c3cccn(Cc4ccc(Cl)nc4)c-3nc2=O)o1] | 1.08 | 385.03 | B |
| A78 | 4-[(6-chloro-3- pyridyl)methyl]-1-thiazol- 2-yl-imidazo[4,5- b]pyridin-2-one | 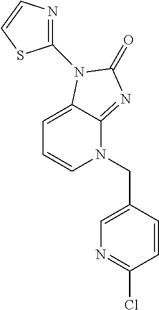 [SMILES: O=c1nc2n(Cc3ccc(Cl)nc3)cccc-2n1-c1nccs1] | 1.11 | 343.98 | B |
| A79 | 4-[(6-chloro-3- pyridyl)methyl]-1-(5- cyclopropyl-1,3,4- oxadiazol-2- yl)imidazo[4,5-b]pyridin- 2-one | 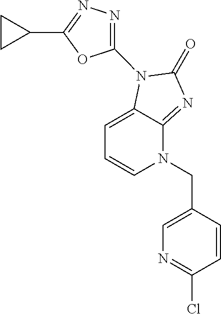 [SMILES: O=c1nc2n(Cc3ccc(Cl)nc3)cccc-2n1-c1nnc(C2CC2)o1] | 0.93 | 369.04 | B |
| A80 | 4-[(6-chloro-3- pyridyl)methyl]-1-[1- (trifluoromethyl)pyrazol- 4-yl]imidazo[4,5- b]pyridin-2-one | 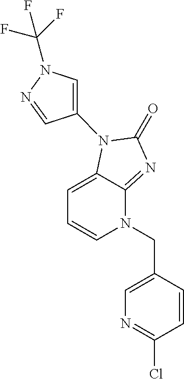 [SMILES: O=c1nc2n(Cc3ccc(Cl)nc3)cccc-2n1-c1cnn(C(F)(F)F)c1] | 1.08 | 395.04 | B |
| A81 | 4-[(6-chloro-3- pyridyl)methyl]-1-[2- (trifluoromethyl)phenyl] imidazo[4,5-b]pyridin-2- one | 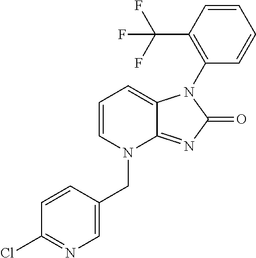 [SMILES: O=c1nc2n(Cc3ccc(Cl)nc3)cccc-2n1-c1ccccc1C(F)(F)F] | ¹H NMR (DMSO-d₆, 400 MHz) δ 8.6 (1H, s), 7.96 (2H, m), 7.92 (1H, s), 7.85 (1H, m), 7.70-7.80 (2H, m), 7.59 (1H, m), 7.28 (1H, d), 6.86 (2H, s) | | |
| A82 | 4-[(6-chloro-3- pyridyl)methyl]-1-(2- methoxyphenyl)imidazo [4,5-b]pyridin-2-one | 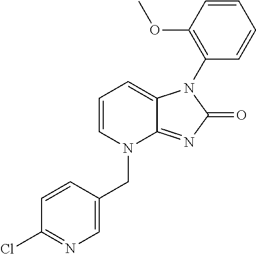 [SMILES: COc1ccccc1-n1c2cccn(Cc3ccc(Cl)nc3)c-2nc1=O] | ¹H NMR (DMSO-d₆, 400 MHz) δ 8.62 (1H, s), 8.02 (1H, d), 7.88 (1H, d), 7.60 (1H, d), 7.45 (1H, t), 7.32 (1H, d), 7.24 (1H, d), 7.08 (1H, t), 6.770-7.78 (2H, m), 5.6 (2H, m) | | |
| A83 | 4-[(6-chloro-3- pyridyl)methyl]-1-(1- propylpyrazol-4- yl)imidazo[4,5-b]pyridin- 2-one | 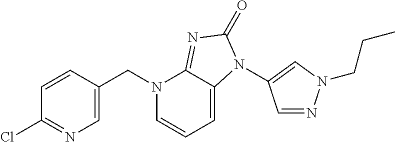 [SMILES: CCCn1cc(-n2c3cccn(Cc4ccc(Cl)nc4)c-3nc2=O)cn1] | 0.71 | 369 | A |
| A84 | 4-[(6-chloro-3- pyridyl)methyl]-1-(1- cyclobutylpyrazol-4- yl)imidazo[4,5-b]pyridin- 2-one | 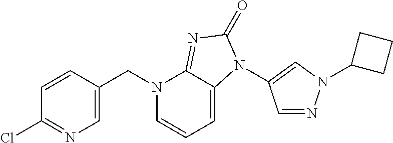 [SMILES: O=c1nc2n(Cc3ccc(Cl)nc3)cccc-2n1-c1cnn(C2CCC2)c1] | 0.74 | 381 | A |
| A85 | 4-[(6-chloro-3- pyridyl)methyl]-1-[1- (2,2,2- trifluoroethyl)pyrazol-4- yl]imidazo[4,5-b]pyridin- 2-one | 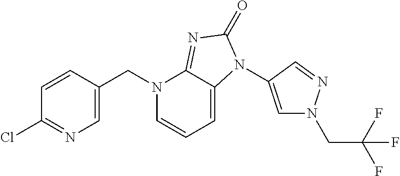 [SMILES: O=c1nc2n(Cc3ccc(Cl)nc3)cccc-2n1-c1cnn(CC(F)(F)F)c1] | 0.72 | 409 | A |
| A86 | 1-(1-butylpyrazol-4-yl)-4- [(6-chloro-3- pyridyl)methyl]imidazo [4,5-b]pyridin-2-one | 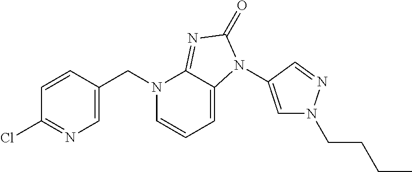 [SMILES: CCCCn1cc(-n2c3cccn(Cc4ccc(Cl)nc4)c-3nc2=O)cn1] | 0.77 | 383 | A |
| A87 | 4-[(6-chloro-3- pyridyl)methyl]-1-[1- (oxetan-3-yl)pyrazol-4- yl]imidazo[4,5-b]pyridin- 2-one | 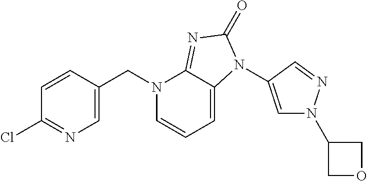 [SMILES: O=c1nc2n(Cc3ccc(Cl)nc3)cccc-2n1-c1cnn(C2COC2)c1] | 0.7 | 337 | A |
| A88 | 4-[(6-chloro-3- pyridyl)methyl]-1-[2- methyl-5- (trifluoromethyl)pyrazol- 3-yl]imidazo[4,5- b]pyridin-2-one | 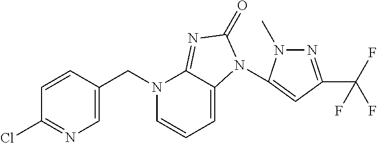 [SMILES: Cn1nc(C(F)(F)F)cc1-n1c2cccn(Cc3ccc(Cl)nc3)c-2nc1=O] | 0.8 | 409 | A |
| A89 | 4-[(6-chloro-3- pyridyl)methyl]-1-(2- ethylpyrazol-3- yl)imidazo[4,5-b]pyridin- 2-one | 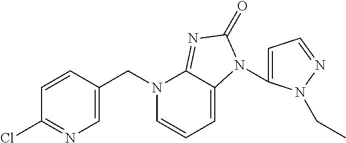 [SMILES: CCn1nccc1-n1c2cccn(Cc3ccc(Cl)nc3)c-2nc1=O] | 0.65 | 355 | A |
| A90 | 4-[(2-chlorothiazol-5- yl)methyl]-5-methyl-1- phenyl-imidazo[4,5- b]pyridin-2-one | 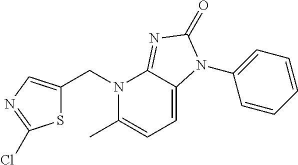 [SMILES: Cc1ccc2n(-c3ccccc3)c(=O)nc-2n1Cc1cnc(Cl)s1] | 0.81 | 357 | A |
| A91 | 4-[(6-chloro-3- pyridyl)methyl]-1-(2- oxotetrahydrofuran-3- yl)imidazo[4,5-b]pyridin- 2-one | 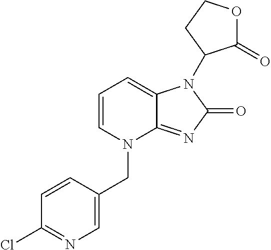 [SMILES: O=C1OCCC1n1c2cccn(Cc3ccc(Cl)nc3)c-2nc1=O] | 0.72 | 345.15 | B |
| A92 | 4-[(6-chloro-3- pyridyl)methyl]-1-(1,5- dimethylpyrazol-4- yl)imidazo[4,5-b]pyridin- 2-one | 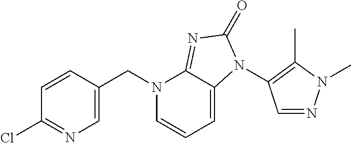 [SMILES: Cc1c(-n2c3cccn(Cc4ccc(Cl)nc4)c-3nc2=O)cnn1C] | 0.64 | 355 | A |
| A93 | 4-[(6-chloro-3- pyridyl)methyl]-1-(1- cyclopropylpyrazol-4- yl)imidazo[4,5-b]pyridin- 2-one | 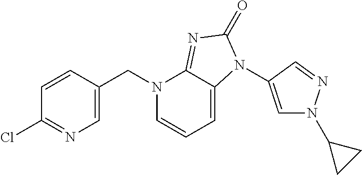 [SMILES: O=c1nc2n(Cc3ccc(Cl)nc3)cccc-2n1-c1cnn(C2CC2)c1] | 0.7 | 367 | A |
| A94 | 4-[(6-chloro-3- pyridyl)methyl]-1-(2- isopropylpyrazol-3- yl)imidazo[4,5-b]pyridin- 2-one | 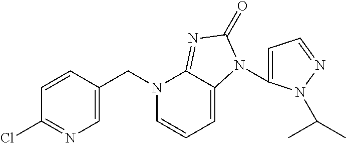 [SMILES: CC(C)n1nccc1-n1c2cccn(Cc3ccc(Cl)nc3)c-2nc1=O] | 0.73 | 369 | A |
| A95 | 4-[(6-chloro-3- pyridyl)methyl]-1-[3- (difluoromethyl)-1- methyl-pyrazol-4- yl]imidazo[4,5-b]pyridin- 2-one | 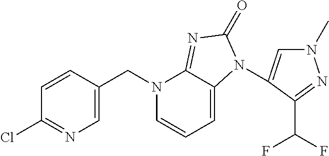 [SMILES: Cn1cc(-n2c3cccn(Cc4ccc(Cl)nc4)c-3nc2=O)c(C(F)F)n1] | 0.69 | 391 | A |
| A96 | 4-[(6-chloro-3- pyridyl)methyl]-1-[1- methyl-3- (trifluoromethyl)pyrazol- 4-yl]imidazo[4,5- b]pyridin-2-one | 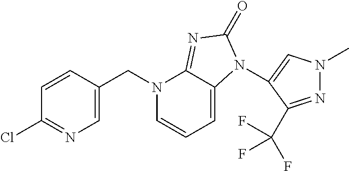 [SMILES: Cn1cc(-n2c3cccn(Cc4ccc(Cl)nc4)c-3nc2=O)c(C(F)(F)F)n1] | 0.75 | 409 | A |
| A97 | 4-[(6-chloro-3- pyridyl)methyl]-1-[5- (difluoromethyl)-1- methyl-pyrazol-4- yl]imidazo[4,5-b]pyridin- 2-one | 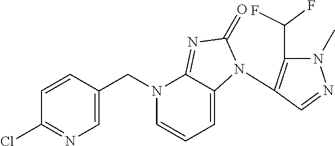 [SMILES: Cn1ncc(-n2c3cccn(Cc4ccc(Cl)nc4)c-3nc2=O)c1C(F)F] | 0.71 | 391 | A |
| A98 | 4-[(6-chloro-3- pyridyl)methyl]-1-[1- (difluoromethyl)-3- methyl-pyrazol-4- yl]imidazo[4,5-b]pyridin- 2-one | 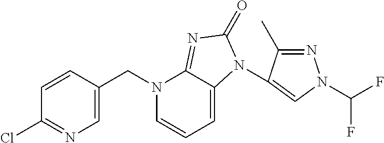 [SMILES: Cc1nn(C(F)F)cc1-n1c2cccn(Cc3ccc(Cl)nc3)c-2nc1=O] | 0.71 | 391 | A |
| A99 | 4-[(6-chloro-3- pyridyl)methyl]-1-[1- (difluoromethyl)-5- methyl-pyrazol-4- yl]imidazo[4,5-b]pyridin- 2-one | 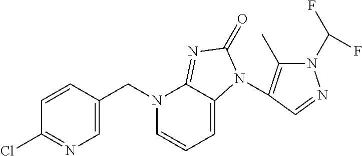 [SMILES: Cc1c(-n2c3cccn(Cc4ccc(Cl)nc4)c-3nc2=O)cnn1C(F)F] | 0.73 | 391 | A |
| A100 | 4-[(6-chloro-3- pyridyl)methyl]-1-(3- ethyl-1-methyl-pyrazol- 4-yl)imidazo[4,5- b]pyridin-2-one | 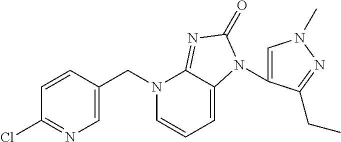 [SMILES: CCc1nn(C)cc1-n1c2cccn(Cc3ccc(Cl)nc3)c-2nc1=O] | 0.68 | 369 | A |
| A101 | 4-[(6-chloro-3- pyridyl)methyl]-1-(1- ethyl-3-methyl-pyrazol- 4-yl)imidazo[4,5- b]pyridin-2-one | 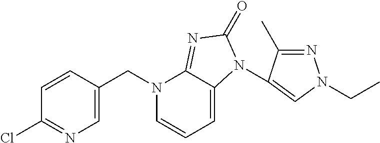 [SMILES: CCn1cc(-n2c3cccn(Cc4ccc(Cl)nc4)c-3nc2=O)c(C)n1] | 0.68 | 369 | A |
| A102 | 4-[(6-chloro-3- pyridyl)methyl]-1-(1- isopropyl-3-methyl- pyrazol-4-yl)imidazo[4,5- b]pyridin-2-one | 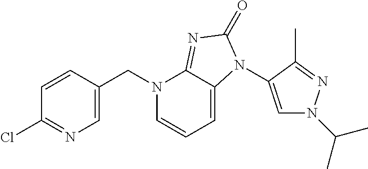 [SMILES: Cc1nn(C(C)C)cc1-n1c2cccn(Cc3ccc(Cl)nc3)c-2nc1=O] | 0.73 | 383 | A |

### BIOLOGICAL EXAMPLES

Bemisia tabaci (Cotton White Fly): Feeding/Contact Activity

Cotton leaf discs were placed on agar in 24-well microtiter plates and sprayed with aqueous test solutions prepared from 10,000 ppm DMSO stock solutions. After drying the leaf discs were infested with adult white flies. The samples were checked for mortality 6 days after incubation.

The following compounds resulted in at least 80% mortality at an application rate of 200 ppm:

A3, A13, A39, A62, A63, A66, A70, A80, A83, A85, A88, A92, A93, A94, A96, A100, A101, A102.

Myzus persicae (Green Peach Aphid): Feeding/Contact Activity

Sunflower leaf discs were placed onto agar in a 24-well microtiter plate and sprayed with aqueous test solutions prepared from 10,000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm:

A1, A2, A3, A5, A6, A7, A8, A9, A10, A11, A13, A14, A21, A23, A25, A26, A29, A30, A32, A33, A34, A36, A38, A39, A40, A42, A62, A63, A66, A67, A79, A80, A81, A82, A83, A85, A87, A88, A92, A93, A94, A95, A96, A97, A98, A99, A100, A101, A102.

Myzus persicae (Green Peach Aphid). Systemic Activity

Roots of pea seedlings infested with an aphid population of mixed ages were placed directly into aqueous test solutions prepared from 10,000 ppm DMSO stock solutions. The samples were assessed for mortality 6 days after placing seedlings into test solutions.

The following compounds resulted in at least 80% mortality at a test rate of 24 ppm:

A2, A3, A16, A22, A24, A25, A26, A27, A31, A32, A33, A34, A40, A82, A83, A85, A88, A92, A93, A94, A95, A96, A97, A98, A99, A100, A102.

Myzus persicae (Green Peach Aphid). Intrinsic Activity

Test compounds prepared from 10,000 ppm DMSO stock solutions were applied by pipette into 24-well microtiter plates and mixed with sucrose solution. The plates were closed with a stretched Parafilm. A plastic stencil with 24 holes was placed onto the plate and infested pea seedlings were placed directly on the Parafilm. The infested plate was closed with a gel blotting paper and another plastic stencil and then turned upside down. The samples were assessed for mortality 5 days after infestation.

The following compounds resulted in at least 80% mortality at a test rate of 12 ppm:

A1, A2, A3, A5, A6, A7, A8, A9, A10, A11, A13, A14, A16, A19, A21, A22, A24, A25, A26, A27, A29, A30, A31, A32, A33, A34, A36, A38, A39, A40, A41, A42, A47, A51, A62, A63, A66, A67, A68, A70, A71, A73, A77, A79, A80, A81, A82, A83, A85, A88, A89, A92, A93, A94, A95, A96, A97, A98, A99, A100, A101, A102.

Nilaparvata lugens (Brown Plant Hopper—Metabolic Neonicotinoid-Resistant), Larvicide, Feeding/Contact

Rice plants were treated with the diluted test solutions in a spray chamber. After drying, the plants were infested with ˜20 N3 nymphs. 7 days after the treatment, samples were assessed for mortality and growth regulation.

The following compounds, according to the present invention, gave at least 80% control of the neonicotinoid-resistant strain of Nilaparvata lugens at 200 ppm.

A3, A31, A33, A34, A92, A93.

Bemisia tabaci (Neonicotinoid-Resistant, Cotton Whitefly-Metabolic Neonicotinoid-Resistant), Adult.

5 cm cotton leaf discs were placed upside down in petri dishes poured out with 11 mL 0.8% water agar and applied in a turntable spraying chamber. After drying of the spray deposits, leaf discs were infested with 10 adult neonicotinoid-resistant Bemisia tabaci. Dishes were covered with a fabric filter and sealed with a perforated plastic lid. Evaluation was made 4 days after infestation on % adult mortality.

The following compounds gave at least 80% control of the neonicotinoid-resistant Bemisia tabaci at 200 ppm.

A63.

Myzus persicae (Green Peach Aphid—Neonicotinoid-Resistant), Mixed Population, Contact

Pea seedlings infested with a mixed-aged neonicotinoid-resistant Myzus persicae population were treated with diluted test solutions in a spray chamber and checked for mortality 5 days after treatment.

The following compounds, according to the present invention, gave at least 80% control of the neonicotinoid-resistant strain of Myzus persicae at 200 ppm.

A26, A30.

Myzus persicae (Neonicotinoid-Resistant, Green Peach Aphid), Mixed Population, Contact/Feeding.

Pepper plants were infested with mixed aged neonicotinoid-resistant aphid population and were treated 1 day after infestation with diluted test solutions in a spray chamber. 5 days after treatment, samples were assessed for mortality.

The following compounds described gave at least 80% control of the neonicotinoid-resistant strain of Myzus persicae at 200 ppm.

A2, A3, A63, A80, A92, A93.

Myzus persicae (Test Method for Resistance Factor 50 (RF(50)).

Cabbage leaf discs were infested with approximately 20-25 insects and sprayed with the respective insecticide dilutions in a Potter Tower. Insect mortality was assessed at five days after treatment.

The RF(50) is calculated by the following formula: RF(50)=LC(50) of resistant strain/LC(50) of susceptible strain, wherein the LC(50) is the lethal concentration where 50% of the population is controlled.

The following compounds, according to the present invention, gave at least a RF(50) lower than or equal to 25.

A3, A26, A30, A34, A80.

1. A compound of Formula (I): 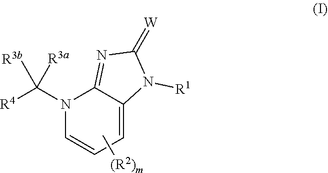
wherein:
W is O or S;
R¹ is phenyl or naphthyl, each optionally: (i) mono- or polysubstituted by a substituent independently selected from U₁ₐ, (ii) mono- or disubstituted by a substituent independently selected from U_{1b}, or (iii) mono- or disubstituted by a substituent independently selected from U₁ₐ and monosubstituted by a substituent selected from U_{1b}; or
R¹ is a 5- to 12-membered heteroaromatic ring system or a 3- to 12-membered saturated or partially saturated heterocyclic ring system, wherein the ring system is monocyclic or polycyclic and comprises 1 to 4 heteroatoms selected from nitrogen, oxygen and sulfur, with the proviso that each ring system cannot contain more than 2 oxygen or sulfur atoms, and wherein each ring system is optionally: (i) mono- or polysubstituted by a substituent independently selected from U₁ₐ, (ii) mono- or disubstituted by a substituent independently selected from U_{1b}, or (iii) mono- or disubstituted by a substituent independently selected from U₁ₐ and monosubstituted by a substituent selected from U_{1b};
U₁ₐ is independently selected from halogen, C₁-C₆alkyl, C₁-C₆haloalkyl, C₁-C₆alkoxy and C₁-C₆haloalkoxy;
U_{1b} is independently selected from nitro, cyano, amino, hydroxyl, —SCN, —CO₂H, C₃-C₆cycloalkyl, C₃-C₆halocycloalkyl, C₃-C₆cycloalkyl-C₁-C₄alkyl, C₃-C₆halocycloalkyl-C₁-C₄alkyl, C₁-C₄alkoxy-C₁-C₄alkyl, C₁-C₄alkoxy-C₁-C₄alkoxy, cyano-C₁-C₄alkyl, cyano-C₁-C₄haloalkyl, C₂-C₆alkenyl, C₂-C₆haloalkenyl, C₂-C₆alkynyl, C₂-C₆haloalkynyl, C₁-C₄haloalkoxy-C₁-C₄alkyl, C₁-C₆alkylsulfanyl, C₁-C₆alkylsulfinyl, C₁-C₆alkylsulfonyl, C₁-C₆haloalkylsulfanyl, C₁-C₆haloalkylsulfinyl, C₁-C₆haloalkylsulfonyl, C₁-C₆alkylcarbonyl, C₁-C₆alkoxy carbonyl, C₁-C₆haloalkylcarbonyl, C₁-C₆haloalkoxycarbonyl, (C₁-C₆alkyl)N(H)—, (C₁-C₆alkyl)₂N—, (C₃-C₆cycloalkyl)N(H)—, (C₃-C₆cycloalkyl)₂N—, C₁-C₆alkylcarbonylamino, C₃-C₆cycloalkylcarbonylamino, C₁-C₆haloalkylcarbonylamino, C₃-C₆halocycloalkylcarbonylamino, C₁-C₆alkylaminocarbonyl, C₃-C₆cycloalkylaminocarbonyl, C₁-C₆haloalkylaminocarbonyl, C₃-C₆halocycloalkylaminocarbonyl, C₃-C₆cycloalkylcarbonyl, C₃-C₆halocycloalkylcarbonyl, —SF₅, —NHS(O)₂ C₁-C₄alkyl, formyl or —C(O)NH₂; or
U_{1b} is phenyl optionally mono- or disubstituted by a group independently selected from U₂; or
U_{1b} is a 5- or 6-membered heteroaromatic ring or a 5- or 6-membered saturated or partially saturated heterocyclic ring, wherein each ring comprises 1 to 4 heteroatoms selected from nitrogen, oxygen and sulfur, with the proviso that each ring cannot contain more than 2 oxygen or sulfur atoms, and wherein each ring is optionally mono- or disubstituted by a group independently selected from U₂;
U² is halogen, C₁-C₆alkyl, C₁-C₆haloalkyl, C₁-C₆alkoxy, C₁-C₆haloalkoxy, nitro, cyano, amino, hydroxyl, —SCN, —CO₂H, C₃-C₆cycloalkyl, C₃-C₆halocycloalkyl, C₃-C₆cycloalkyl-C₁-C₄alkyl, C₃-C₆halocycloalkyl-C₁-C₄alkyl, C₁-C₄alkoxy-C₁-C₄alkyl, C₁-C₄alkoxy-C₁-C₄alkoxy, cyano-C₁-C₄alkyl, cyano-C₁-C₄haloalkyl, C₂-C₆alkenyl, C₂-C₆haloalkenyl, C₂-C₆alkynyl, C₂-C₆haloalkynyl, C₁-C₄haloalkoxy-C₁-C₄alkyl, C₁-C₆alkylsulfanyl, C₁-C₆alkylsulfinyl, C₁-C₆alkylsulfonyl, C₁-C₆haloalkylsulfanyl, C₁-C₆haloalkylsulfinyl, C₁-C₆haloalkylsulfonyl, C₁-C₆alkylcarbonyl, C₁-C₆alkoxycarbonyl, C₁-C₆haloalkylcarbonyl, C₁-C₆haloalkoxycarbonyl, —SF₅ or —C(O)NH₂;
m is 0, 1 or 2;
R² is independently selected from halogen, cyano, amino, hydroxyl, C₁-C₆alkyl, C₁-C₆haloalkyl, C₁-C₆haloalkoxy, C₁-C₆alkoxy, C₂-C₆alkenyl, C₂-C₆haloalkenyl, C₂-C₆alkynyl, C₂-C₆haloalkynyl, C₃-C₆cycloalkyl, C₃-C₆halocycloalkyl, C₁-C₆alkylsulfanyl, C₁-C₆alkylsulfinyl, C₁-C₆alkylsulfonyl, C₁-C₆haloalkylsulfanyl, C₁-C₆haloalkylsulfinyl and C₁-C₆haloalkylsulfonyl;
R^{3a} and R^{3b} are independently selected from hydrogen, halogen, C₁-C₄alkyl, C₁-C₄haloalkyl, C₁-C₄alkoxy, C₁-C₄haloalkoxy and cyano;
R₄ is selected from one of Y1 to Y7; 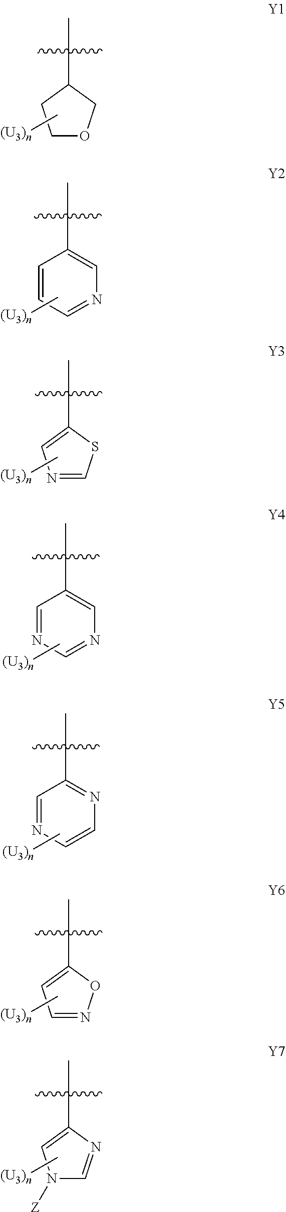
wherein, n is 0, 1, 2, or 3;
Z is hydrogen, C₁-C₄alkyl, C₁-C₄haloalkyl, C₁-C₄alkoxy or C₁-C₄haloalkoxy; and
U₃ is independently selected from halogen, cyano, nitro, hydroxyl, amino, C₁-C₄alkyl, C₁-C₄haloalkyl, C₁-C₄alkoxy, C₁-C₄haloalkoxy, C₁-C₄haloalkoxy-C₁-C₄alkyl, C₁-C₄alkoxy-C₁-C₄alkyl, C₁-C₄alkylsulfanyl, C₁-C₄alkylsulfinyl, C₁-C₄alkylsulfonyl, C₁-C₄haloalkylsulfanyl, C₁-C₄haloalkylsulfinyl, C₁-C₄haloalkylsulfonyl, formyl, cyclopropyl, C₁-C₆alkylcarbonyl or C₃-C₆cycloalkylcarbonyl;
or an agrochemically acceptable salt, stereoisomer, enantiomer, tautomer or N-oxide thereof.

2. The compound according to claim 1, wherein R¹ is phenyl, or a 5- or 6-membered heteroaromatic monocyclic ring system, which ring system comprises 1 or 2 nitrogen atoms or is furanyl or thienyl, wherein each R¹ is optionally:
(i) mono- or disubstituted by a substituent independently selected from U₁ₐ,
(ii) mono- or disubstituted by a substituent independently selected from U_{1b}, or
(iii) mono- or disubstituted by a substituent independently selected from U₁ₐ and monosubstituted by a substituent selected from U_{1b}.

3. The compound according to claim 1, wherein R¹ is phenyl, pyrazolyl, pyridinyl, pyrimidinyl, pyridazinyl, pyrazinyl, oxazolyl, oxadiazolyl, thiazolyl, thiadiazolyl, furanyl or thienyl, wherein R¹ is optionally substituted by:
(i) 1 or 2 substituents independently selected from U₁ₐ, wherein U₁ₐ is halogen, C₁-C₄alkyl, C₁-C₄fluoroalkyl, C₁-C₄alkoxy and C₁-C₄fluoroalkoxy, or
(ii) 1 substituent selected from U_{1b}, wherein U_{1b} is nitro, cyano, amino, C₃-C₆cycloalkyl, cyanoC₁-C₄alkyl, C₁-C₄alkylcarbonyl, C₁-C₄alkoxycarbonyl, C₁-C₄haloalkylcarbonyl, or U_{1b} is phenyl or oxetan-3-yl optionally substituted by a substituent selected from U₂, wherein U₂ is fluoro, chloro, methyl, ethyl, methoxy, ethoxy or trifluoromethyl.

4. The compound according to claim 1, wherein R¹ is pyrazolyl optionally substituted by 1 or 2 substituents independently selected from U₁ₐ, wherein U₁ₐ is C₁-C₄alkyl or C₁-C₄fluoroalkyl, or a single substituent selected from U_{1b}, wherein U_{1b} is C₃-C₄cycloalkyl.

5. The compound according to claim 1, wherein m is 0 or 1.

6. The compound according to claim 1, wherein R^{3a} is hydrogen and R^{3b} is hydrogen.

7. The compound according to claim 1, wherein R⁴ is selected from Y2, Y3 or Y4.

8. The compound according to claim 1, wherein n is 0 or 1.

9. The compound according to claim 1, wherein R⁴ is selected from one of:

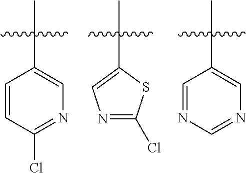

10. The compound according to claim 1, wherein R⁴ is:

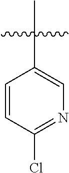 [SMILES: *c1ccc(Cl)nc1]

11. A compound of Formula (II): 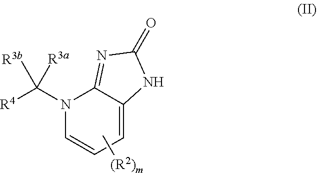
wherein m, R², R^{3a}, R^{3b} and R⁴ correspond to the same definitions as for the compounds of Formula (I) as defined in claim 1,
with the proviso that the compound of Formula (II) is not 4-[(6-chloro-3-pyridyl)methyl]-1H-imidazo[4,5-b]pyridin-2-one.

12. An agrochemical composition comprising an insecticidally, acaricidally, nematicidally or molluscicidally effective amount of a compound according to claim 1.

13. The composition according to claim 12, further comprising at least one additional active ingredient and/or an agrochemically-acceptable diluent or carrier.

14. A method of controlling insects, acarines, nematodes or molluscs which comprises applying an insecticidally, acaricidally, nematicidally or molluscicidally effective amount of a compound of formula (I) as defined in claim 1, to a pest, a locus of pest (preferably a plant), to a plant susceptible to attack by a pest or to plant a propagation material thereof (such as a seed).

15. Use of a compound according to claim 1 as an insecticide, acaricide, nematicide or molluscicide.

16. The compound according to claim 5, wherein m is 0.
